(19) United States
(12) Patent Application Publication
(10) Pub. No.: US 2022/0135690 A1
(43) Pub. Date: May 5, 2022

(54) METHODS AND COMPOSITIONS RELATING TO CHEMOKINE RECEPTOR VARIANTS

(71) Applicant: Twist Bioscience Corporation, South San Francisco, CA (US)

(72) Inventors: Aaron SATO, Burlingame, CA (US); Qiang LIU, Palo Alto, CA (US); Linya WANG, Milpitas, CA (US); Fumiko AXELROD, Palo Alto, CA (US)

(21) Appl. No.: 17/517,528

(22) Filed: Nov. 2, 2021

Related U.S. Application Data
(60) Provisional application No. 63/109,280, filed on Nov. 3, 2020.

(51) Int. Cl. C07K 16/28; A61P 35/00

(52) U.S. Cl. CPC C07K 16/2866; A61P 35/00; A61K 2039/505; C07K 2317/92; C07K 2317/24; C07K 2317/565

(57) ABSTRACT

Provided herein are methods and compositions relating to chemokine receptor libraries having nucleic acids encoding for immunoglobulins that bind to chemokine receptors. Libraries described herein include variegated libraries comprising nucleic acids each encoding for a predetermined variant of at least one predetermined reference nucleic acid sequence. Further described herein are protein libraries generated when the nucleic acid libraries are translated. Further described herein are cell libraries expressing variegated nucleic acid libraries described herein.

### CROSS-REFERENCE

This application claims the benefit of U.S. Provisional Patent Application No. 63/109,280 filed on Nov. 3, 2020, which is incorporated by reference in its entirety.

### BACKGROUND

G protein-coupled receptors (GPCRs) are implicated in a wide variety of diseases. Raising antibodies to GPCRs has been difficult due to problems in obtaining suitable antigen because GPCRs are often expressed at low levels in cells and are very unstable when purified. Thus, there is a need for improved agents for therapeutic intervention which target GPCRs.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are antibodies or antibody fragments comprising a variable domain, heavy chain region (VH) and a variable domain, light chain region (VL), wherein VH comprises complementarity determining regions CDRH1, CDRH2, and CDRH3, wherein VL comprises complementarity determining regions CDRL1, CDRL2, and CDRL3, and wherein (a) an amino acid sequence of CDRH1 is as set forth in any one of SEQ ID NOs: 526-662; (b) an amino acid sequence of CDRH2 is as set forth in any one of SEQ ID NOs: 663-977; (c) an amino acid sequence of CDRH3 is as set forth in any one of SEQ ID NOs: 978-1102; (d) an amino acid sequence of CDRL1 is as set forth in any one of SEQ ID NOs: 1103-1267; (e) an amino acid sequence of CDRL2 is as set forth in any one of SEQ ID NOs: 1268-1328; and (f) an amino acid sequence of CDRL3 is as set forth in any one of SEQ ID NOs: 1329-1493. Further provided herein are antibodies or antibody fragments, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. Further provided herein are antibodies or antibody fragments, wherein the antibody or antibody fragment thereof is chimeric or humanized. Further provided herein are antibodies or antibody fragments, wherein the antibody or antibody fragment has an EC50 less than about 25 nanomolar in a cAMP assay. Further provided herein are antibodies or antibody fragments, wherein the antibody or antibody fragment has an EC50 less than about 20 nanomolar in a cAMP assay. Further provided herein are antibodies or antibody fragments, wherein the antibody or antibody fragment has an EC50 less than about 10 nanomolar in a cAMP assay. Further provided herein are antibodies or antibody fragments, wherein the antibody or antibody fragment binds to a chemokine receptor with a K_{D} of less than 100 nM. Further provided herein are antibodies or antibody fragments, wherein the antibody or antibody fragment binds to a chemokine receptor with a K_{D} of less than 75 nM. Further provided herein are antibodies or antibody fragments, wherein the antibody or antibody fragment binds to a chemokine receptor with a K_{D} of less than 50 nM. Further provided herein are antibodies or antibody fragments, wherein the antibody or antibody fragment binds to a chemokine receptor with a K_{D} of less than 10 nM. Further provided herein are antibodies or antibody fragments, wherein the antibody or antibody fragment is an agonist of a chemokine receptor. Further provided herein are antibodies or antibody fragments, wherein the antibody or antibody fragment is an antagonist of a chemokine receptor. Further provided herein are antibodies or antibody fragments, wherein the antibody or antibody fragment is an allosteric modulator of a chemokine receptor. Further provided herein are antibodies or antibody fragments, wherein the allosteric modulator of a chemokine receptor is a negative allosteric modulator. Further provided herein are antibodies or antibody fragments, wherein the chemokine receptor is CXCR4. Further provided herein are antibodies or antibody fragments, wherein the chemokine receptor is CXCR5.

Provided herein are antibodies or antibody fragments comprising a variable domain, heavy chain region (VH) and a variable domain, light chain region (VL), wherein the VH comprises an amino acid sequence at least about 90% identical to a sequence as set forth in any one of SEQ ID NOs: 24-28 or 34-356, and wherein the VL comprises an amino acid sequence at least about 90% identical to a sequence as set forth in any one of SEQ ID NOs: 29-33 or 357-525. Further provided herein are antibodies or antibody fragments, wherein the VH comprises an amino acid sequence as set forth in any one of SEQ ID NOs: 24-28 or 34-356. Further provided herein are antibodies or antibody fragments, wherein the VL comprises an amino acid sequence as set forth in any one of SEQ ID NOs: 29-33 or 357-525. Further provided herein are antibodies or antibody fragments, wherein the VH comprises an amino acid sequence at least about 90% identical to a sequence as set forth in any one of SEQ ID NOs: 24-28 and the VL comprises an amino acid sequence at least about 90% identical to a sequence as set forth in any one of SEQ ID NOs: 29-33. Further provided herein are antibodies or antibody fragments, wherein the VH comprises an amino acid sequence as set forth in any one of SEQ ID NOs: 24-28 and the VL comprises an amino acid sequence as set forth in any one of SEQ ID NOs: 29-33. Further provided herein are antibodies or antibody fragments, wherein the VH comprises an amino acid sequence at least about 90% identical to a sequence as set forth in any one of SEQ ID NOs: 34-356 and the VL comprises an amino acid sequence at least about 90% identical to a sequence as set forth in any one of SEQ ID NOs: 357-525. Further provided herein are antibodies or antibody fragments, wherein the VH comprises an amino acid sequence as set forth in any one of SEQ ID NOs: 34-356 and the VL comprises an amino acid sequence as set forth in any one of SEQ ID NOs: 357-525. Further provided herein are antibodies or antibody fragments, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarily determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. Further provided herein are antibodies or antibody fragments, wherein the antibody or antibody fragment thereof is chimeric or humanized. Further provided herein are antibodies or antibody fragments, wherein the antibody or antibody fragment has an EC50 less than about 25 nanomolar in a cAMP assay. Further provided herein are antibodies or antibody fragments, wherein the antibody or antibody fragment has an EC50 less than about 20 nanomolar in a cAMP assay. Further provided herein are antibodies or antibody fragments, wherein the antibody or antibody fragment has an EC50 less than about 10 nanomolar in a cAMP assay. Further provided herein are antibodies or antibody fragments, wherein the antibody or antibody fragment binds to a chemokine receptor with a K_{D} of less than 100 nM. Further provided herein are antibodies or antibody fragments, wherein the antibody or antibody fragment binds to a chemokine receptor with a K_{D} of less than 75 nM. Further provided herein are antibodies or antibody fragments, wherein the antibody or antibody fragment binds to a chemokine receptor with a K_{D} of less than 50 nM. Further provided herein are antibodies or antibody fragments, wherein the antibody or antibody fragment binds to a chemokine receptor with a K_{D} of less than 10 nM. Further provided herein are antibodies or antibody fragments, wherein the antibody or antibody fragment is an agonist of a chemokine receptor. Further provided herein are antibodies or antibody fragments, wherein the antibody or antibody fragment is an antagonist of a chemokine receptor. Further provided herein are antibodies or antibody fragments, wherein the antibody or antibody fragment is an allosteric modulator of a chemokine receptor. Further provided herein are antibodies or antibody fragments, wherein the allosteric modulator of a chemokine receptor is a negative allosteric modulator. Further provided herein are antibodies or antibody fragments, wherein the chemokine receptor is CXCR4. Further provided herein are antibodies or antibody fragments, wherein the chemokine receptor is CXCR5.

Provided herein are antibodies or antibody fragments comprising a variable domain, heavy chain region (VH) comprising an amino acid sequence at least about 90% identical to a sequence as set forth in any one of SEQ ID NOs: 24-28 or 34-356. Further provided herein are antibodies or antibody fragments, wherein the VH comprises an amino acid sequence as set forth in any one of SEQ ID NOs: 24-28 or 34-356. Further provided herein are antibodies or antibody fragments, wherein the VH comprises an amino acid sequence at least about 90% identical to a sequence as set forth in any one of SEQ ID NOs: 24-28. Further provided herein are antibodies or antibody fragments, wherein the VH comprises an amino acid sequence as set forth in any one of SEQ ID NOs: 34-356. Further provided herein are antibodies or antibody fragments, wherein the VH comprises an amino acid sequence at least about 90% identical to a sequence as set forth in any one of SEQ ID NOs: 24-28. Further provided herein are antibodies or antibody fragments, wherein the VH comprises an amino acid sequence as set forth in any one of SEQ ID NOs: 34-356. Further provided herein are antibodies or antibody fragments, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarily determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. Further provided herein are antibodies or antibody fragments, wherein the antibody is a single-domain antibody. Further provided herein are antibodies or antibody fragments, wherein the antibody or antibody fragment thereof is chimeric or humanized. Further provided herein are antibodies or antibody fragments, wherein the antibody or antibody fragment has an EC50 less than about 25 nanomolar in a cAMP assay. Further provided herein are antibodies or antibody fragments, wherein the antibody or antibody fragment has an EC50 less than about 20 nanomolar in a cAMP assay. Further provided herein are antibodies or antibody fragments, wherein the antibody or antibody fragment has an EC50 less than about 10 nanomolar in a cAMP assay. Further provided herein are antibodies or antibody fragments, wherein the antibody or antibody fragment binds to a chemokine receptor with a K_{D} of less than 100 nM. Further provided herein are antibodies or antibody fragments, wherein the antibody or antibody fragment binds to a chemokine receptor with a K_{D} of less than 75 nM. Further provided herein are antibodies or antibody fragments, wherein the antibody or antibody fragment binds to a chemokine receptor with a K_{D} of less than 50 nM. Further provided herein are antibodies or antibody fragments, wherein the antibody or antibody fragment binds to a chemokine receptor with a K_{D} of less than 10 nM. Further provided herein are antibodies or antibody fragments, wherein the antibody or antibody fragment is an agonist of a chemokine receptor. Further provided herein are antibodies or antibody fragments, wherein the antibody or antibody fragment is an antagonist of a chemokine receptor. Further provided herein are antibodies or antibody fragments, wherein the antibody or antibody fragment is an allosteric modulator of a chemokine receptor. Further provided herein are antibodies or antibody fragments, wherein the allosteric modulator of a chemokine receptor is a negative allosteric modulator. Further provided herein are antibodies or antibody fragments, wherein the chemokine receptor is CXCR4. Further provided herein are antibodies or antibody fragments, wherein the chemokine receptor is CXCR5.

Provided herein are methods of treating a disease or disorder comprising administering the antibody or antibody fragment described herein. Further provided herein are methods of treating a disease or disorder, wherein the disease or disorder affects homeostasis. Further provided herein are methods of treating a disease or disorder, wherein the disease or disorder characterized by hematopoietic stem cell migration. Further provided herein are methods of treating a disease or disorder, wherein the disease or disorder is a solid cancer or a hematologic cancer. Further provided herein are methods of treating a disease or disorder, wherein the disease or disorder is gastric cancer, breast cancer, colorectal cancer, lung cancer, prostate cancer, hepatocellular carcinoma, leukemia, or lymphoma. Further provided herein are methods of treating a disease or disorder, wherein the disease or disorder is B-cell non-Hodgkin lymphoma. Further provided herein are methods of treating a disease or disorder, wherein the disease or disorder is caused by a virus. Further provided herein are methods of treating a disease or disorder, wherein the disease or disorder is caused by human immunodeficiency virus (HIV).

Provided herein are nucleic acid compositions comprising: a) a first nucleic acid encoding a variable domain, heavy chain region (VH) comprising complementarity determining regions CDRH1, CDRH2, and CDRH3, and wherein (i) an amino acid sequence of CDRH1 is as set forth in any one of SEQ ID NOs: 526-662; (ii) an amino acid sequence of CDRH2 is as set forth in any one of SEQ ID NOs: 663-977; (iii) an amino acid sequence of CDRH3 is as set forth in any one of SEQ ID NOs: 978-1102; b) a second nucleic acid encoding a variable domain, light chain region (VL) comprising complementarity determining regions CDRL1, CDRL2, and CDRL3, and wherein (i) an amino acid sequence of CDRL1 is as set forth in any one of SEQ ID NOs: 1103-1267; (ii) an amino acid sequence of CDRL2 is as set forth in any one of SEQ ID NOs: 1268-1328; and (iii) an amino acid sequence of CDRL3 is as set forth in any one of SEQ ID NOs: 1329-1493.

Provided herein are nucleic acid compositions comprising: a) a first nucleic acid encoding a variable domain, heavy chain region (VH) comprising an amino acid sequence at least about 90% identical to a sequence as set forth in any one of SEQ ID NOs: 24-28 or 34-356; b) a second nucleic acid encoding a variable domain, light chain region (VL) comprising at least about 90% identical to a sequence as set forth in any one of SEQ ID NOs: 29-33 or 357-525; and an excipient. Further provided herein are nucleic acid compositions, wherein the VH comprises an amino acid sequence as set forth in any one of SEQ ID NOs: 24-28 or 34-356. Further provided herein are nucleic acid compositions, wherein the VL comprises an amino acid sequence as set forth in any one of SEQ ID NOs: 29-33 or 357-525. Further provided herein are nucleic acid compositions, wherein the VH comprises an amino acid sequence at least about 90% identical to a sequence as set forth in any one of SEQ ID NOs: 24-28 and the VL comprises an amino acid sequence at least about 90% identical to a sequence as set forth in any one of SEQ ID NOs: 29-33. Further provided herein are nucleic acid compositions, wherein the VH comprises an amino acid sequence as set forth in any one of SEQ ID NOs: 24-28 and the VL comprises an amino acid sequence as set forth in any one of SEQ ID NOs: 29-33. Further provided herein are nucleic acid compositions, wherein the VH comprises an amino acid sequence at least about 90% identical to a sequence as set forth in any one of SEQ ID NOs: 34-356 and the VL comprises an amino acid sequence at least about 90% identical to a sequence as set forth in any one of SEQ ID NOs: 357-525. Further provided herein are nucleic acid compositions, wherein the VH comprises an amino acid sequence as set forth in any one of SEQ ID NOs: 34-356 and the VL comprises an amino acid sequence as set forth in any one of SEQ ID NOs: 357-525.

Provided herein are nucleic acid compositions comprising: a nucleic acid encoding a variable domain, heavy chain region (VH) comprising an amino acid sequence at least about 90% identical to a sequence as set forth in any one of SEQ ID NOs: 24-28 or 34-356; and an excipient. Further provided herein are nucleic acid compositions, wherein the VH comprises an amino acid sequence as set forth in any one of SEQ ID NOs: 24-28 or 34-356. Further provided herein are nucleic acid compositions, wherein the VH comprises an amino acid sequence at least about 90% identical to a sequence as set forth in any one of SEQ ID NOs: 24-28. Further provided herein are nucleic acid compositions, wherein the VH comprises an amino acid sequence as set forth in any one of SEQ ID NOs: 34-356. Further provided herein are nucleic acid compositions, wherein the VH comprises an amino acid sequence at least about 90% identical to a sequence as set forth in any one of SEQ ID NOs: 24-28. Further provided herein are nucleic acid compositions, wherein the VH comprises an amino acid sequence as set forth in any one of SEQ ID NOs: 34-356.

Provided herein are nucleic acid libraries, comprising: a plurality of nucleic acids, wherein each of the nucleic acids encodes for a sequence that when translated encodes for a chemokine receptor binding immunoglobulin, wherein the chemokine receptor binding immunoglobulin comprises a variant of a chemokine receptor binding domain, wherein the chemokine receptor binding domain is a ligand for the chemokine receptor, and wherein the nucleic acid library comprises at least 10,000 variant immunoglobulin heavy chains and at least 10,000 variant immunoglobulin light chains. Further provided are nucleic acid libraries, wherein the nucleic acid library comprises at least 50,000 variant immunoglobulin heavy chains and at least 50,000 variant immunoglobulin light chains. Further provided are nucleic acid libraries, wherein the nucleic acid library comprises at least 100,000 variant immunoglobulin heavy chains and at least 100,000 variant immunoglobulin light chains. Further provided are nucleic acid libraries, wherein the nucleic acid library comprises at least 10⁵ non-identical nucleic acids. Further provided are nucleic acid libraries, wherein a length of the immunoglobulin heavy chain when translated is about 90 to about 100 amino acids. Further provided are nucleic acid libraries, wherein a length of the immunoglobulin heavy chain when translated is about 100 to about 400 amino acids. Further provided are nucleic acid libraries, wherein the variant immunoglobulin heavy chain when translated comprises at least 80% sequence identity to any one of SEQ ID NOs: 24-28 or 34-356. Further provided are nucleic acid libraries, wherein the variant immunoglobulin light chain when translated comprises at least 80% sequence identity to any one of SEQ ID NOs: 29-33 or 357-525.

Provided herein are nucleic acid libraries comprising a plurality of nucleic acids, wherein each nucleic acid of the plurality of nucleic acids encodes for a sequence that when translated encodes for an antibody or antibody fragment thereof, wherein the antibody or antibody fragment thereof comprises a variable region of a heavy chain (VH) that comprises a chemokine receptor binding domain, wherein each nucleic acid of the plurality of nucleic acids comprises a sequence encoding for a sequence variant of the chemokine receptor binding domain, and wherein the antibody or antibody fragment binds to its antigen with a K_{D} of less than 100 nM. Further provided are nucleic acid libraries, wherein a length of the VH is about 90 to about 100 amino acids. Further provided are nucleic acid libraries, wherein a length of the VH is about 100 to about 400 amino acids. Further provided are nucleic acid libraries, wherein a length of the VH is about 270 to about 300 base pairs. Further provided are nucleic acid libraries, wherein a length of the VH is about 300 to about 1200 base pairs. Further provided are nucleic acid libraries, wherein the library comprises at least 10⁵ non-identical nucleic acids.

Provided herein are nucleic acid libraries comprising: a plurality of nucleic acids, wherein each of the nucleic acids encodes for a sequence that when translated encodes for a chemokine receptor single domain antibody, wherein each sequence of the plurality of sequences comprises a variant sequence encoding for a CDR1, CDR2, or CDR3 on a variable region of a heavy chain (VH); wherein the library comprises at least 30,000 variant sequences; and wherein the chemokine receptor single domain antibody binds to its antigen with a K_{D} of less than 100 nM. Further provided are nucleic acid libraries, wherein a length of the VH when translated is about 90 to about 100 amino acids. Further provided are nucleic acid libraries, wherein a length of the VH when translated is about 100 to about 400 amino acids. Further provided are nucleic acid libraries, wherein a length of the VH is about 270 to about 300 base pairs. Further provided are nucleic acid libraries, wherein a length of the VH is about 300 to about 1200 base pairs. Further provided are nucleic acid libraries, wherein the VH when translated comprises at least 80% sequence identity to any one of SEQ ID NO: 24-28 or 34-356.

Provided herein are antibodies or antibody fragments that binds chemokine receptor, comprising an immunoglobulin heavy chain and an immunoglobulin light chain: (a) wherein the immunoglobulin heavy chain comprises an amino acid sequence at least about 90% identical to that set forth in any one of SEQ ID NO: 24-28 or 34-356; and (b) wherein the immunoglobulin light chain comprises an amino acid sequence at least about 90% identical to that set forth in any one of SEQ ID NO: 29-33 or 357-525. Further provided herein are antibodies or antibody fragments, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. Further provided herein are antibodies or antibody fragments, wherein the antibody or antibody fragment thereof is chimeric or humanized. Further provided herein are antibodies or antibody fragments, wherein the antibody has an EC50 less than about 25 nanomolar in a cAMP assay. Further provided herein are antibodies or antibody fragments, wherein the antibody has an EC50 less than about 20 nanomolar in a cAMP assay. Further provided herein are antibodies or antibody fragments, wherein the antibody has an EC50 less than about 10 nanomolar in a cAMP assay.

Provided herein are antibodies or antibody fragments, wherein the antibody or antibody fragment comprises a complementarity determining region (CDR) comprising an amino acid sequence at least about 90% identical to that set forth in any one of SEQ ID NOs: 526-1493.

Provided herein are antibodies or antibody fragments, wherein the antibody or antibody fragment comprises a sequence of any one of SEQ ID NOs: 526-1493 and wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

Provided herein are methods for generating a nucleic acid library encoding for a chemokine receptor antibody or antibody fragment thereof comprising: (a) providing predetermined sequences encoding for: i. a first plurality of polynucleotides, wherein each polynucleotide of the first plurality of polynucleotides encodes for at least 1000 variant sequence encoding for CDR1 on a heavy chain; ii. a second plurality of polynucleotides, wherein each polynucleotide of the second plurality of polynucleotides encodes for at least 1000 variant sequence encoding for CDR2 on a heavy chain; iii. a third plurality of polynucleotides, wherein each polynucleotide of the third plurality of polynucleotides encodes for at least 1000 variant sequence encoding for CDR3 on a heavy chain; and (b) mixing the first plurality of polynucleotides, the second plurality of polynucleotides, and the third plurality of polynucleotides to form the nucleic acid library of variant nucleic acids encoding for the chemokine receptor antibody or antibody fragment thereof, and wherein at least about 70% of the variant nucleic acids encode for an antibody or antibody fragment that binds to its antigen with a K_{D} of less than 100 nM. Further provided herein are methods, wherein the chemokine receptor antibody or antibody fragment thereof is a single domain antibody. Further provided herein are methods, wherein the single domain antibody comprises one heavy chain variable domain. Further provided herein are methods, wherein the single domain antibody is a VHH antibody. Further provided herein are methods, wherein the nucleic acid library comprises at least 50,000 variant sequences. Further provided herein are methods, wherein the nucleic acid library comprises at least 100,000 variant sequences. Further provided herein are methods, wherein the nucleic acid library comprises at least 10⁵ non-identical nucleic acids. Further provided herein are methods, wherein the nucleic acid library comprises at least one sequence encoding for the chemokine receptor antibody or antibody fragment that binds to chemokine receptor with a K_{D} of less than 75 nM. Further provided herein are methods, wherein the nucleic acid library comprises at least one sequence encoding for the chemokine receptor antibody or antibody fragment that binds to chemokine receptor with a K_{D} of less than 50 nM. Further provided herein are methods, wherein the nucleic acid library comprises at least one sequence encoding for the chemokine receptor antibody or antibody fragment that binds to chemokine receptor with a K_{D} of less than 10 nM. Further provided herein are methods, wherein the nucleic acid library comprises at least 500 variant sequences. Further provided herein are methods, wherein the nucleic acid library comprises at least five sequences encoding for the chemokine receptor antibody or antibody fragment that binds to chemokine receptor with a K_{D} of less than 75 nM.

Provided herein are protein libraries encoded by the nucleic acid library described herein, wherein the protein library comprises peptides. Further provided herein are protein libraries, wherein the protein library comprises immunoglobulins. Further provided herein are protein libraries, wherein the protein library comprises antibodies. Further provided herein are protein libraries, wherein the protein library is a peptidomimetic library.

Provided herein are vector libraries comprising the nucleic acid library described herein.

Provided herein are cell libraries comprising the nucleic acid library described herein.

Provided herein are cell libraries comprising the protein library described herein.

### BRIEF DESCRIPTION OF THE DRAWINGS

FIG. 1A depicts a first schematic of an immunoglobulin scaffold.

FIG. 1B depicts a second schematic of an immunoglobulin scaffold.

FIG. 2 depicts a schematic of a motif for placement in a scaffold.

FIG. 3 presents a diagram of steps demonstrating an exemplary process workflow for gene synthesis as disclosed herein.

FIG. 4 illustrates an example of a computer system.

FIG. 5 is a block diagram illustrating an architecture of a computer system.

FIG. 6 is a diagram demonstrating a network configured to incorporate a plurality of computer systems, a plurality of cell phones and personal data assistants, and Network Attached Storage (NAS).

FIG. 7 is a block diagram of a multiprocessor computer system using a shared virtual address memory space.

FIG. 8A depicts a schematic of an immunoglobulin scaffold comprising a VH domain attached to a VL domain using a linker.

FIG. 8B depicts a schematic of a full-domain architecture of an immunoglobulin scaffold comprising a VH domain attached to a VL domain using a linker, a leader sequence, and pill sequence.

FIG. 8C depicts a schematic of four framework elements (FW1, FW2, FW3, FW4) and the variable 3 CDR (L1, L2, L3) elements for a VL or VH domain.

FIG. 9A depicts a structure of Glucagon-like peptide 1 (GLP-1, cyan) in complex with GLP-1 receptor (GLP-1R, grey), PDB entry 5VAI.

FIG. 9B depicts a crystal structure of CXCR4 chemokine receptor (grey) in complex with a cyclic peptide antagonist CVX15 (blue), PDB entry 3OR0.

FIG. 9C depicts a crystal structure of human smoothened with the transmembrane domain in grey and extracellular domain (ECD) in orange, PDB entry 5L7D. The ECD contacts the TMD through extracellular loop 3 (ECL3).

FIG. 9D depicts a structure of GLP-1R (grey) in complex with a Fab (magenta), PDB entry 6LN2.

FIG. 9E depicts a crystal structure of CXCR4 (grey) in complex with a viral chemokine antagonist Viral macrophage inflammatory protein 2 (vMIP-II, green), PDB entry 4RWS.

FIG. 10 depicts a schema of the GPCR focused library design. Two germline heavy chain VH1-69 and VH3-30; 4 germline light chain IGKV1-39 and IGKV3-15, and IGLV1-51 and IGLV2-14.

FIG. 11 depicts a graph of HCDR3 length distribution in the GPCR-focused library compared to the HCDR3 length distribution in B-cell populations from three healthy adult donors. In total, 2,444,718 unique VH sequences from the GPCR library and 2,481,511 unique VH sequences from human B-cell repertoire were analyzed to generate the length distribution plot.

FIG. 12 depicts a graph of data from CXCR4 variants in a titration assay.

FIG. 13 depicts exemplary CXCR4 variant sequences.

FIG. 14A depicts a graph of data from CXCR4 variants in an allosteric cAMP peptide assay.

FIG. 14B depicts a graph of data from CXCR4 variants in an antagonistic cAMP peptide assay.

FIG. 15A depicts a graph showing ligand titrations of CXCR4 variants determined using Homogeneous Time Resolved Fluorescence (HTRF).

FIG. 15B depicts a graph of different ligand titrations of CXCR4 variants.

FIG. 15C depicts a graph of peptide/IgG ligand titrations with CXCR4 variants determined using HTRF.

FIG. 15D depicts a graph of different peptide/IgG ligand titrations with CXCR4 variants.

FIG. 16A depicts data from flow titration assays using variant CXCR4-81-6.

FIG. 16B depicts a graph of a cAMP assay using variant CXCR4-81-6.

FIGS. 17A-17D depict graphs of data from CXCR5 variants in a titration assay.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred herein may comprise at least one region encoding for exon sequences without an intervening intron sequence in the genomic equivalent sequence.

GPCR Libraries for Chemokine Receptor

Provided herein are methods and compositions relating to G protein-coupled receptor (GPCR) binding libraries for chemokine receptor comprising nucleic acids encoding for a scaffold comprising a GPCR binding domain. Scaffolds as described herein can stably support a GPCR binding domain. The GPCR binding domain may be designed based on surface interactions of a chemokine receptor ligand and a chemokine receptor. In some instances, the chemokine receptor is CXCR5 receptor. In some instances, the chemokine receptor is CXCR4 receptor. Libraries as described herein may be further variegated to provide for variant libraries comprising nucleic acids each encoding for a predetermined variant of at least one predetermined reference nucleic acid sequence. Further described herein are protein libraries that may be generated when the nucleic acid libraries are translated. In some instances, nucleic acid libraries as described herein are transferred into cells to generate a cell library. Also provided herein are downstream applications for the libraries synthesized using methods described herein. Downstream applications include identification of variant nucleic acids or protein sequences with enhanced biologically relevant functions, e.g., improved stability, affinity, binding, functional activity, and for the treatment or prevention of a disease state associated with GPCR signaling.

Scaffold Libraries

Provided herein are libraries comprising nucleic acids encoding for a scaffold, wherein sequences for GPCR binding domains are placed in the scaffold. Scaffold described herein allow for improved stability for a range of GPCR binding domain encoding sequences when inserted into the scaffold, as compared to an unmodified scaffold. Exemplary scaffolds include, but are not limited to, a protein, a peptide, an immunoglobulin, derivatives thereof, or combinations thereof. In some instances, the scaffold is an immunoglobulin. Scaffolds as described herein comprise improved functional activity, structural stability, expression, specificity, or a combination thereof. In some instances, scaffolds comprise long regions for supporting a GPCR binding domain.

Provided herein are libraries comprising nucleic acids encoding for a scaffold, wherein the scaffold is an immunoglobulin. In some instances, the immunoglobulin is an antibody. As used herein, the term antibody will be understood to include proteins having the characteristic two-armed, Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CH1 domains), a F(ab′)2 fragment (including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for a scaffold, wherein the scaffold is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for a scaffold, wherein the scaffold is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass.

In some embodiments, libraries comprise immunoglobulins that are adapted to the species of an intended therapeutic target. Generally, these methods include “mammalization” and comprises methods for transferring donor antigen-binding information to a less immunogenic mammal antibody acceptor to generate useful therapeutic treatments. In some instances, the mammal is mouse, rat, equine, sheep, cow, primate (e.g., chimpanzee, baboon, gorilla, orangutan, monkey), dog, cat, pig, donkey, rabbit, and human. In some instances, provided herein are libraries and methods for felinization and caninization of antibodies.

“Humanized” forms of non-human antibodies can be chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. In some instances, these modifications are made to further refine antibody performance.

“Caninization” can comprise a method for transferring non-canine antigen-binding information from a donor antibody to a less immunogenic canine antibody acceptor to generate treatments useful as therapeutics in dogs. In some instances, caninized forms of non-canine antibodies provided herein are chimeric antibodies that contain minimal sequence derived from non-canine antibodies. In some instances, caninized antibodies are canine antibody sequences (“acceptor” or “recipient” antibody) in which hypervariable region residues of the recipient are replaced by hypervariable region residues from a non-canine species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the canine antibody are replaced by corresponding non-canine FR residues. In some instances, caninized antibodies include residues that are not found in the recipient antibody or in the donor antibody. In some instances, these modifications are made to further refine antibody performance. The caninized antibody may also comprise at least a portion of an immunoglobulin constant region (Fc) of a canine antibody.

“Felinization” can comprise a method for transferring non-feline antigen-binding information from a donor antibody to a less immunogenic feline antibody acceptor to generate treatments useful as therapeutics in cats. In some instances, felinized forms of non-feline antibodies provided herein are chimeric antibodies that contain minimal sequence derived from non-feline antibodies. In some instances, felinized antibodies are feline antibody sequences (“acceptor” or “recipient” antibody) in which hypervariable region residues of the recipient are replaced by hypervariable region residues from a non-feline species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the feline antibody are replaced by corresponding non-feline FR residues. In some instances, felinized antibodies include residues that are not found in the recipient antibody or in the donor antibody. In some instances, these modifications are made to further refine antibody performance. The felinized antibody may also comprise at least a portion of an immunoglobulin constant region (Fc) of a felinize antibody.

Provided herein are libraries comprising nucleic acids encoding for a scaffold, wherein the scaffold is a non-immunoglobulin. In some instances, the scaffold is a non-immunoglobulin binding domain. For example, the scaffold is an antibody mimetic. Exemplary antibody mimetics include, but are not limited to, anticalins, affilins, affibody molecules, affimers, affitins, alphabodies, avimers, atrimers, DARPins, fynomers, Kunitz domain-based proteins, monobodies, anticalins, knottins, armadillo repeat protein-based proteins, and bicyclic peptides.

Libraries described herein comprising nucleic acids encoding for a scaffold, wherein the scaffold is an immunoglobulin, comprise variations in at least one region of the immunoglobulin. Exemplary regions of the antibody for variation include, but are not limited to, a complementarity-determining region (CDR), a variable domain, or a constant domain. In some instances, the CDR is CDR1, CDR2, or CDR3. In some instances, the CDR is a heavy domain including, but not limited to, CDRH1, CDRH2, and CDRH3. In some instances, the CDR is a light domain including, but not limited to, CDRL1, CDRL2, and CDRL3. In some instances, the variable domain is variable domain, light chain (VL) or variable domain, heavy chain (VH). In some instances, the VL domain comprises kappa or lambda chains. In some instances, the constant domain is constant domain, light chain (CL) or constant domain, heavy chain (CH).

Methods described herein provide for synthesis of libraries comprising nucleic acids encoding for a scaffold, wherein each nucleic acid encodes for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the scaffold library comprises varied nucleic acids collectively encoding variations at multiple positions. In some instances, the variant library comprises sequences encoding for variation of at least a single codon of a CDRH1, CDRH2, CDRH3, CDRL1, CDRL2, CDRL3, VL, or VH domain. In some instances, the variant library comprises sequences encoding for variation of multiple codons of a CDRH1, CDRH2, CDRH3, CDRL1, CDRL2, CDRL3, VL, or VH domain. In some instances, the variant library comprises sequences encoding for variation of multiple codons of framework element 1 (FW1), framework element 2 (FW2), framework element 3 (FW3), or framework element 4 (FW4). An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

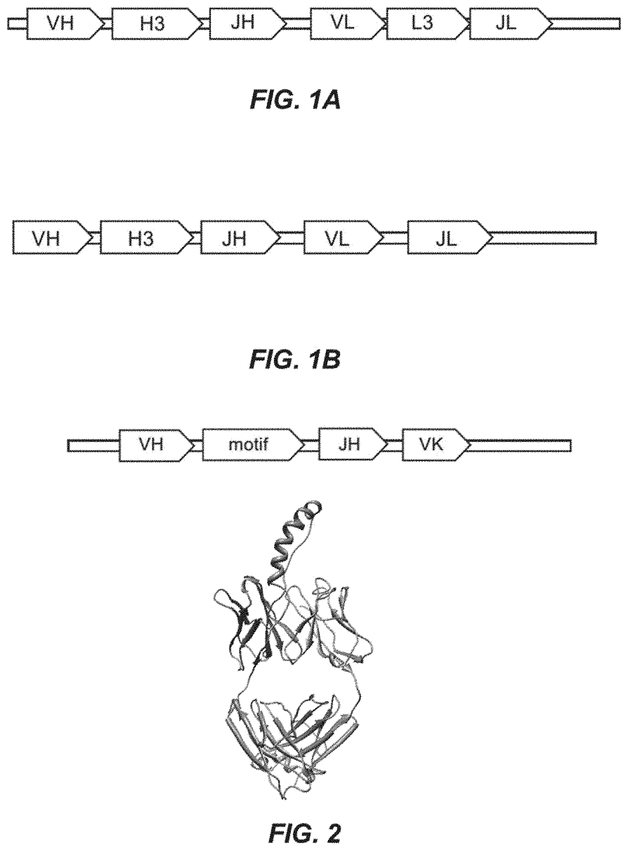

In some instances, the at least one region of the immunoglobulin for variation is from heavy chain V-gene family, heavy chain D-gene family, heavy chain J-gene family, light chain V-gene family, or light chain J-gene family. See FIGS. 1A-1B. In some instances, the light chain V-gene family comprises immunoglobulin kappa (IGK) gene or immunoglobulin lambda (IGL). Exemplary genes include, but are not limited to, IGHV1-18, IGHV1-69, IGHV1-8, IGHV3-21, IGHV3-23, IGHV3-30/33m, IGHV3-28, IGHV1-69, IGHV3-74, IGHV4-39, IGHV4-59/61, IGKV1-39, IGKV1-9, IGKV2-28, IGKV3-11, IGKV3-15, IGKV3-20, IGKV4-1, IGLV1-51, IGLV2-14, IGLV1-40, and IGLV3-1. In some instances, the gene is IGKJ1, IGKJ4, or IGKJ2. In some instances, the gene is IGKV1 or IGKV2. In some instances, the gene is IGHV1-69, IGHV3-30, IGHV3-23, IGHV3, IGHV1-46, IGHV3-7, IGHV1, or IGHV1-8. In some instances, the gene is IGHV1 or IGHV3. In some instances, the gene is IGHV1-69 and IGHV3-30. In some instances, the gene is IGHJ3, IGHJ6, IGHJ, IGHJ4, IGHJ5, IGHJ2, or IGH1. In some instances, the gene is IGHJ3, IGHJ6, IGHJ, or IGHJ4. In some instances, the gene is IGHJ2, IGHJ4, IGHJ5, or IGHJ6.

Provided herein are libraries comprising nucleic acids encoding for immunoglobulin scaffolds, wherein the libraries are synthesized with various numbers of fragments. In some instances, the fragments comprise the CDRH1, CDRH2, CDRH3, CDRL1, CDRL2, CDRL3, VL, or VH domain. In some instances, the fragments comprise framework element 1 (FW1), framework element 2 (FW2), framework element 3 (FW3), or framework element 4 (FW4). In some instances, the scaffold libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

Libraries comprising nucleic acids encoding for immunoglobulin scaffolds as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the immunoglobulin scaffolds comprise at least or about 100, 200, 300, 400, 500, 600, 700, 800, 900, 1000, 2000, 3000, 4000, 5000, or more than 5000 amino acids.

A number of variant sequences for the at least one region of the immunoglobulin for variation are de novo synthesized using methods as described herein. In some instances, a number of variant sequences is de novo synthesized for CDRH1, CDRH2, CDRH3, CDRL1, CDRL2, CDRL3, VL, VH, or combinations thereof. In some instances, a number of variant sequences is de novo synthesized for framework element 1 (FW1), framework element 2 (FW2), framework element 3 (FW3), or framework element 4 (FW4). The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is at least or about 500, 600, 700, 800, 900, 1000, 2000, 3000, 4000, 5000, 6000, 7000, 8000, or more than 8000 sequences. In some instances, the number of variant sequences is about 10 to 500, 25 to 475, 50 to 450, 75 to 425, 100 to 400, 125 to 375, 150 to 350, 175 to 325, 200 to 300, 225 to 375, 250 to 350, or 275 to 325 sequences.

Variant sequences for the at least one region of the immunoglobulin, in some instances, vary in length or sequence. In some instances, the at least one region that is de novo synthesized is for CDRH1, CDRH2, CDRH3, CDRL1, CDRL2, CDRL3, VL, VH, or combinations thereof. In some instances, the at least one region that is de novo synthesized is for framework element 1 (FW1), framework element 2 (FW2), framework element 3 (FW3), or framework element 4 (FW4). In some instances, the variant sequence comprises at least or about 1, 2, 3, 4, 5, 6, 7, 8, 9, 10, 15, 20, 25, 30, 35, 40, 45, 50, or more than 50 variant nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 additional nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 less nucleotides or amino acids as compared to wild-type. In some instances, the libraries comprise at least or about 10¹, 10², 10³, 10⁴, 10⁵, 10⁶, 10⁷, 10⁸, 10⁹, 10¹⁰, or more than 10¹⁰ variants.

Following synthesis of scaffold libraries, scaffold libraries may be used for screening and analysis. For example, scaffold libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, scaffold libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

In some instances, the scaffold libraries are assayed for functional activity, structural stability (e.g., thermal stable or pH stable), expression, specificity, or a combination thereof. In some instances, the scaffold libraries are assayed for scaffolds capable of folding. In some instances, a region of the antibody is assayed for functional activity, structural stability, expression, specificity, folding, or a combination thereof. For example, a VH region or VL region is assayed for functional activity, structural stability, expression, specificity, folding, or a combination thereof

Chemokine Receptor Libraries

Provided herein are chemokine receptor binding libraries comprising nucleic acids encoding for scaffolds comprising sequences for chemokine receptor binding domains. In some instances, the scaffolds are immunoglobulins. In some instances, the scaffolds comprising sequences for chemokine receptor binding domains are determined by interactions between the chemokine receptor binding domains and the chemokine receptor.

Provided herein are libraries comprising nucleic acids encoding scaffolds comprising chemokine receptor binding domains, wherein the chemokine receptor binding domains are designed based on surface interactions on chemokine receptor. In some instances, the chemokine receptor comprises a sequence as defined by SEQ ID NO: 1. In some instances, the chemokine receptor binding domains interact with the amino- or carboxy-terminus of the chemokine receptor. In some instances, the chemokine receptor binding domains interact with at least one transmembrane domain including, but not limited to, transmembrane domain 1 (TM1), transmembrane domain 2 (TM2), transmembrane domain 3 (TM3), transmembrane domain 4 (TM4), transmembrane domain 5 (TM5), transmembrane domain 6 (TM6), and transmembrane domain 7 (TM7). In some instances, the chemokine receptor binding domains interact with an intracellular surface of the chemokine receptor. For example, the chemokine receptor binding domains interact with at least one intracellular loop including, but not limited to, intracellular loop 1 (ICL1), intracellular loop 2 (ICL2), and intracellular loop 3 (ICL3). In some instances, the chemokine receptor binding domains interact with an extracellular surface of the chemokine receptor. For example, the chemokine receptor binding domains interact with at least one extracellular domain (ECD) or extracellular loop (ECL) of the chemokine receptor. The extracellular loops include, but are not limited to, extracellular loop 1 (ECL1), extracellular loop 2 (ECL2), and extracellular loop 3 (ECL3).

Described herein are chemokine receptor binding domains, wherein the chemokine receptor binding domains are designed based on surface interactions between a chemokine receptor ligand and the chemokine receptor. In some instances, the ligand is a peptide. In some instances, the ligand is CXCL12, migration inhibitory factor (MIF), extracellular Ubiquitin (eUb), Gp120, vMIP-II, or human β3-defensin. In some instances, the ligand is CXCL12-α, CXCL12-β, CXCL12-γ, CXCL12-δ, CXCL12-ε, or CXCL12-φ. In some instances, the ligand is CXCL13. In some instances, the ligand is a chemokine receptor agonist. In some instances, the ligand is a chemokine receptor antagonist. In some instances, the ligand is a chemokine receptor allosteric modulator. In some instances, the allosteric modulator is a negative allosteric modulator. In some instances, the allosteric modulator is a positive allosteric modulator.

Sequences of chemokine receptor binding domains based on surface interactions between a chemokine receptor ligand and the chemokine receptor are analyzed using various methods. For example, multispecies computational analysis is performed. In some instances, a structure analysis is performed. In some instances, a sequence analysis is performed. Sequence analysis can be performed using a database known in the art. Non-limiting examples of databases include, but are not limited to, NCBI BLAST (blast.ncbi.nlm.nih.gov/Blast.cgi), UCSC Genome Browser (genome.ucsc.edu/), UniProt (www.uniprot.org/), and IUPHAR/BPS Guide to PHARMACOLOGY (guidetopharmacology.org/).

Described herein are chemokine receptor binding domains designed based on sequence analysis among various organisms. For example, sequence analysis is performed to identify homologous sequences in different organisms. Exemplary organisms include, but are not limited to, mouse, rat, equine, sheep, cow, primate (e.g., chimpanzee, baboon, gorilla, orangutan, monkey), dog, cat, pig, donkey, rabbit, fish, fly, and human.

Following identification of chemokine receptor binding domains, libraries comprising nucleic acids encoding for the chemokine receptor binding domains may be generated. In some instances, libraries of chemokine receptor binding domains comprise sequences of chemokine receptor binding domains designed based on conformational ligand interactions, peptide ligand interactions, small molecule ligand interactions, extracellular domains of chemokine receptor, or antibodies that target chemokine receptor. In some instances, libraries of chemokine receptor binding domains comprise sequences of chemokine receptor binding domains designed based on peptide ligand interactions. Libraries of chemokine receptor binding domains may be translated to generate protein libraries. In some instances, libraries of chemokine receptor binding domains are translated to generate peptide libraries, immunoglobulin libraries, derivatives thereof, or combinations thereof. In some instances, libraries of chemokine receptor binding domains are translated to generate protein libraries that are further modified to generate peptidomimetic libraries. In some instances, libraries of chemokine receptor binding domains are translated to generate protein libraries that are used to generate small molecules.

Methods described herein provide for synthesis of libraries of chemokine receptor binding domains comprising nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the libraries of chemokine receptor binding domains comprise varied nucleic acids collectively encoding variations at multiple positions. In some instances, the variant library comprises sequences encoding for variation of at least a single codon in a chemokine receptor binding domain. In some instances, the variant library comprises sequences encoding for variation of multiple codons in a chemokine receptor binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of libraries comprising nucleic acids encoding for the chemokine receptor binding domains, wherein the libraries comprise sequences encoding for variation of length of the chemokine receptor binding domains. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following identification of chemokine receptor binding domains, the chemokine receptor binding domains may be placed in scaffolds as described herein. In some instances, the scaffolds are immunoglobulins. In some instances, the chemokine receptor binding domains are placed in the CDRH3 region. GPCR binding domains that may be placed in scaffolds can also be referred to as a motif. Scaffolds comprising chemokine receptor binding domains may be designed based on binding, specificity, stability, expression, folding, or downstream activity. In some instances, the scaffolds comprising chemokine receptor binding domains enable contact with the chemokine receptor. In some instances, the scaffolds comprising chemokine receptor binding domains enables high affinity binding with the chemokine receptor. An exemplary amino acid sequence of chemokine receptor binding domain is described in Table 1.

TABLE 1
Chemokine amino acid sequences

| SEQ | | |
|---|---|---|
| ID NO | GPCR | Amino Acid Sequence |
| 1 | CXCR4 | MEGISSIPLPLLQIYTSDNYTEEMGSGDYDSMKEPCFREENANFNKIFL |
| | | PTIYSIIFLTGIVGNGLVILVMGYQKKLRSMTDKYRLHLSVADLLFVIT |
| | | LPFWAVDAVANWYFGNFLCKAVHVIYTVNLYSSVLILAFISLDRYLAI |
| | | VHATNSQRPRKLLAEKVVYVGVWIPALLLTIPDFIFANVSEADDRYIC |
| | | DRFYPNDLWVVVFQFQHIMVGLILPGIVILSCYCIIISKLSHSKGHQKR |
| | | KALKTTVILILAFFACWLPYYIGISIDSFILLEIIKQGCEFENTVHKWISIT |
| | | EALAFFHCCLNPILYAFLGAKFKTSAQHALTSVSRGSSLKILSKGKRG |
| | | GHSSVSTESESSSFHSS |
| 2 | CXCR5 | MNYPLTLEMDLENLEDLFWELDRLDNYNDTSLVENHLCPATEGPLM |
| | | ASFKAVFVPVAYSLIFLLGVIGNVLVLVILERHRQTRSSTETFLFHLAV |
| | | ADLLLVFILPFAVAEGSVGWVLGTFLCKTVIALHKVNFYCSSLLLACI |
| | | AVDRYLAIVHAVHAYRHRRLLSIHITCGTIWLVGFLLALPEILFAKVS |
| | | QGHHNNSLPRCTFSQENQAETHAWFTSRFLYHVAGFLLPMLVMGWC |
| | | YVGVVHRLRQAQRRPQRQKAVRVAILVTSIFFLCWSPYHIVIFLDTLA |
| | | RLKAVDNTCKLNGSLPVAITMCEFLGLAHCCLNPMLYTFAGVKFRSD |
| | | LSRLLTKLGCTGPASLCQLFPGWRRSSLSESENATSLTTF |

Provided herein are scaffolds comprising chemokine receptor binding domains, wherein the sequences of the chemokine receptor binding domains support interaction with chemokine receptor. The sequence may be homologous or identical to a sequence of a chemokine receptor ligand. In some instances, the chemokine receptor binding domain sequence comprises at least or about 70%, 80%, 85%, 90%, 91%, 92%, 93%, 94%, 95%, 96%, 97%, 98%, 99%, or 100% sequence identity to SEQ ID NO: 1 or 2. In some instances, the chemokine receptor binding domain sequence comprises at least or about 95% homology to SEQ ID NO: 1 or 2. In some instances, the chemokine receptor binding domain sequence comprises at least or about 97% homology to SEQ ID NO: 1 or 2. In some instances, the chemokine receptor binding domain sequence comprises at least or about 99% homology to SEQ ID NO: 1 or 2. In some instances, the chemokine receptor binding domain sequence comprises at least or about 100% homology to SEQ ID NO: 1. In some instances, the chemokine receptor binding domain sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more than 400 amino acids of SEQ ID NO: 1 or 2.

Described herein, in some embodiments, are antibodies or immunoglobulins that bind to the chemokine receptor. In some embodiments, the chemokine receptor is CXCR4. In some embodiments, the chemokine receptor is CXCR5. In some instances, the chemokine receptor antibody or immunoglobulin sequence comprises a heavy chain variable domain comprising at least or about 70%, 80%, 85%, 90%, 91%, 92%, 93%, 94%, 95%, 96%, 97%, 98%, 99%, or 100% sequence identity to any one of SEQ ID NO: 24-28 or 34-356. In some instances, the chemokine receptor antibody or immunoglobulin sequence comprises a heavy chain variable domain comprising at least or about 95% sequence identity to any one of SEQ ID NO: 24-28 or 34-356. In some instances, the chemokine receptor antibody or immunoglobulin sequence comprises a heavy chain variable domain comprising at least or about 97% sequence identity to any one of SEQ ID NO: 24-28 or 34-356. In some instances, the chemokine receptor antibody or immunoglobulin sequence comprises a heavy chain variable domain comprising at least or about 99% sequence identity to any one of SEQ ID NO: 24-28 or 34-356. In some instances, the chemokine receptor antibody or immunoglobulin sequence comprises a heavy chain variable domain comprising at least or about 100% sequence identity to any one of SEQ ID NO: 24-28 or 34-356. In some instances, the chemokine receptor antibody or immunoglobulin sequence comprises a heavy chain variable domain comprising at least a portion having at least or about 1, 2, 3, 4, 5, 6, 7, 8, 9, 10, 12, 14, 16, 18, 20, 30, 40, 50, 60, 70, 80, 90, 100, 110, 120, or more than 120 amino acids of any one of SEQ ID NO: 24-28 or 34-356.

In some instances, the chemokine receptor antibody or immunoglobulin sequence comprises a heavy chain variable domain comprising at least or about 70%, 80%, 85%, 90%, 91%, 92%, 93%, 94%, 95%, 96%, 97%, 98%, 99%, or 100% sequence identity to any one of SEQ ID NO: 24-28. In some instances, the chemokine receptor antibody or immunoglobulin sequence comprises a heavy chain variable domain comprising at least or about 95% sequence identity to any one of SEQ ID NO: 24-28. In some instances, the chemokine receptor antibody or immunoglobulin sequence comprises a heavy chain variable domain comprising at least or about 97% sequence identity to any one of SEQ ID NO: 24-28. In some instances, the chemokine receptor antibody or immunoglobulin sequence comprises a heavy chain variable domain comprising at least or about 99% sequence identity to any one of SEQ ID NO: 24-28. In some instances, the chemokine receptor antibody or immunoglobulin sequence comprises a heavy chain variable domain comprising at least or about 100% sequence identity to any one of SEQ ID NO: 24-28. In some instances, the chemokine receptor antibody or immunoglobulin sequence comprises a heavy chain variable domain comprising at least a portion having at least or about 1, 2, 3, 4, 5, 6, 7, 8, 9, 10, 12, 14, 16, 18, 20, 30, 40, 50, 60, 70, 80, 90, 100, 110, 120, or more than 120 amino acids of any one of SEQ ID NO: 24-28.

In some instances, the chemokine receptor antibody or immunoglobulin sequence comprises a heavy chain variable domain comprising at least or about 70%, 80%, 85%, 90%, 91%, 92%, 93%, 94%, 95%, 96%, 97%, 98%, 99%, or 100% sequence identity to any one of SEQ ID NO: 34-356. In some instances, the chemokine receptor antibody or immunoglobulin sequence comprises a heavy chain variable domain comprising at least or about 95% sequence identity to any one of SEQ ID NO: 34-356. In some instances, the chemokine receptor antibody or immunoglobulin sequence comprises a heavy chain variable domain comprising at least or about 97% sequence identity to any one of SEQ ID NO: 34-356. In some instances, the chemokine receptor antibody or immunoglobulin sequence comprises a heavy chain variable domain comprising at least or about 99% sequence identity to any one of SEQ ID NO: 34-356. In some instances, the chemokine receptor antibody or immunoglobulin sequence comprises a heavy chain variable domain comprising at least or about 100% sequence identity to any one of SEQ ID NO: 34-356. In some instances, the chemokine receptor antibody or immunoglobulin sequence comprises a heavy chain variable domain comprising at least a portion having at least or about 1, 2, 3, 4, 5, 6, 7, 8, 9, 10, 12, 14, 16, 18, 20, 30, 40, 50, 60, 70, 80, 90, 100, 110, 120, or more than 120 amino acids of any one of SEQ ID NO: 34-356.

In some instances, the chemokine receptor antibody or immunoglobulin sequence comprises a light chain variable domain comprising at least or about 70%, 80%, 85%, 90%, 91%, 92%, 93%, 94%, 95%, 96%, 97%, 98%, 99%, or 100% sequence identity to any one of SEQ ID NO: 29-33 or 357-525. In some instances, the chemokine receptor antibody or immunoglobulin sequence comprises a light chain variable domain comprising at least or about 95% sequence identity to any one of SEQ ID NO: 29-33 or 357-525. In some instances, the chemokine receptor antibody or immunoglobulin sequence comprises a light chain variable domain comprising at least or about 97% sequence identity to any one of SEQ ID NO: 29-33 or 357-525. In some instances, the chemokine receptor antibody or immunoglobulin sequence comprises a light chain variable domain comprising at least or about 99% sequence identity to any one of SEQ ID NO: 29-33 or 357-525. In some instances, the chemokine receptor antibody or immunoglobulin sequence comprises a light chain variable domain comprising at least or about 100% sequence identity to any one of SEQ ID NO: 29-33 or 357-525. In some instances, the chemokine receptor antibody or immunoglobulin sequence comprises a light chain variable domain comprising at least a portion having at least or about 1, 2, 3, 4, 5, 6, 7, 8, 9, 10, 12, 14, 16, 18, 20, 30, 40, 50, 60, 70, 80, 90, 100, 110, or more than 110 amino acids of any one of SEQ ID NO: 29-33 or 357-525.

In some instances, the chemokine receptor antibody or immunoglobulin sequence comprises a light chain variable domain comprising at least or about 70%, 80%, 85%, 90%, 91%, 92%, 93%, 94%, 95%, 96%, 97%, 98%, 99%, or 100% sequence identity to any one of SEQ ID NO: 29-33. In some instances, the chemokine receptor antibody or immunoglobulin sequence comprises a light chain variable domain comprising at least or about 95% sequence identity to any one of SEQ ID NO: 29-33. In some instances, the chemokine receptor antibody or immunoglobulin sequence comprises a light chain variable domain comprising at least or about 97% sequence identity to any one of SEQ ID NO: 29-33. In some instances, the chemokine receptor antibody or immunoglobulin sequence comprises a light chain variable domain comprising at least or about 99% sequence identity to any one of SEQ ID NO: 29-33. In some instances, the chemokine receptor antibody or immunoglobulin sequence comprises a light chain variable domain comprising at least or about 100% sequence identity to any one of SEQ ID NO: 29-33. In some instances, the chemokine receptor antibody or immunoglobulin sequence comprises a light chain variable domain comprising at least a portion having at least or about 1, 2, 3, 4, 5, 6, 7, 8, 9, 10, 12, 14, 16, 18, 20, 30, 40, 50, 60, 70, 80, 90, 100, 110, or more than 110 amino acids of any one of SEQ ID NO: 29-33.

In some instances, the chemokine receptor antibody or immunoglobulin sequence comprises a light chain variable domain comprising at least or about 70%, 80%, 85%, 90%, 91%, 92%, 93%, 94%, 95%, 96%, 97%, 98%, 99%, or 100% sequence identity to any one of SEQ ID NO: 357-525. In some instances, the chemokine receptor antibody or immunoglobulin sequence comprises a light chain variable domain comprising at least or about 95% sequence identity to any one of SEQ ID NO: 357-525. In some instances, the chemokine receptor antibody or immunoglobulin sequence comprises a light chain variable domain comprising at least or about 97% sequence identity to any one of SEQ ID NO: 357-525. In some instances, the chemokine receptor antibody or immunoglobulin sequence comprises a light chain variable domain comprising at least or about 99% sequence identity to any one of SEQ ID NO: 357-525. In some instances, the chemokine receptor antibody or immunoglobulin sequence comprises a light chain variable domain comprising at least or about 100% sequence identity to any one of SEQ ID NO: 357-525. In some instances, the chemokine receptor antibody or immunoglobulin sequence comprises a light chain variable domain comprising at least a portion having at least or about 1, 2, 3, 4, 5, 6, 7, 8, 9, 10, 12, 14, 16, 18, 20, 30, 40, 50, 60, 70, 80, 90, 100, 110, or more than 110 amino acids of any one of SEQ ID NO: 357-525.

Described herein, in some embodiments, are antibodies or antibody fragments comprising a variable domain, heavy chain region (VH) and a variable domain, light chain region (VL), wherein the VH comprises an amino acid sequence at least about 90% identical to a sequence as set forth in any one of SEQ ID NOs: 24-28 or 34-356, and wherein the VL comprises an amino acid sequence at least about 90% identical to a sequence as set forth in any one of SEQ ID NOs: 29-33 or 357-525. In some instances, the antibodies or antibody fragments comprise VH comprising at least or about 70%, 80%, 85%, 90%, 91%, 92%, 93%, 94%, 95%, 96%, 97%, 98%, 99%, or 100% sequence identity to any one of SEQ ID NOs: 24-28 or 34-356, and VL comprising at least or about 70%, 80%, 85%, 90%, 91%, 92%, 93%, 94%, 95%, 96%, 97%, 98%, 99%, or 100% sequence identity to any one of SEQ ID NOs: 29-33 or 357-525.

Described herein, in some embodiments, are antibodies or antibody fragments comprising a variable domain, heavy chain region (VH) and a variable domain, light chain region (VL), wherein the VH comprises an amino acid sequence at least about 90% identical to a sequence as set forth in any one of SEQ ID NOs: 24-28, and wherein the VL comprises an amino acid sequence at least about 90% identical to a sequence as set forth in any one of SEQ ID NOs: 29-33. In some instances, the antibodies or antibody fragments comprise VH comprising at least or about 70%, 80%, 85%, 90%, 91%, 92%, 93%, 94%, 95%, 96%, 97%, 98%, 99%, or 100% sequence identity to any one of SEQ ID NOs: 24-28, and VL comprising at least or about 70%, 80%, 85%, 90%, 91%, 92%, 93%, 94%, 95%, 96%, 97%, 98%, 99%, or 100% sequence identity to any one of SEQ ID NOs: 29-33.

Described herein, in some embodiments, are antibodies or antibody fragments comprising a variable domain, heavy chain region (VH) and a variable domain, light chain region (VL), wherein the VH comprises an amino acid sequence at least about 90% identical to a sequence as set forth in any one of SEQ ID NOs: 34-356, and wherein the VL comprises an amino acid sequence at least about 90% identical to a sequence as set forth in any one of SEQ ID NOs: 357-525. In some instances, the antibodies or antibody fragments comprise VH comprising at least or about 70%, 80%, 85%, 90%, 91%, 92%, 93%, 94%, 95%, 96%, 97%, 98%, 99%, or 100% sequence identity to any one of SEQ ID NOs: 34-356, and VL comprising at least or about 70%, 80%, 85%, 90%, 91%, 92%, 93%, 94%, 95%, 96%, 97%, 98%, 99%, or 100% sequence identity to any one of SEQ ID NOs: 357-525.

In some embodiments, the chemokine receptor antibody or immunoglobulin sequence comprises complementarity determining regions (CDRs) comprising at least or about 70%, 80%, 85%, 90%, 91%, 92%, 93%, 94%, 95%, 96%, 97%, 98%, 99%, or 100% sequence identity to any one of SEQ ID NOs: 526-1102. In some instances, the chemokine receptor antibody or immunoglobulin sequence comprises complementarity determining regions (CDRs) comprising at least or about 95% homology to any one of SEQ ID NOs: 526-1102. In some instances, the chemokine receptor antibody or immunoglobulin sequence comprises complementarity determining regions (CDRs) comprising at least or about 97% homology to any one of SEQ ID NOs: 526-1102. In some instances, the chemokine receptor antibody or immunoglobulin sequence comprises complementarity determining regions (CDRs) comprising at least or about 99% homology to any one of SEQ ID NOs: 526-1102. In some instances, the chemokine receptor antibody or immunoglobulin sequence comprises complementarity determining regions (CDRs) comprising at least or about 100% homology to any one of SEQ ID NOs: 526-1102. In some instances, the chemokine receptor antibody or immunoglobulin sequence comprises complementarity determining regions (CDRs) comprising at least a portion having at least or about 3, 4, 5, 6, 7, 8, 9, 10, 12, 14, 16, 17, 18, 19, 20 or more than 20 amino acids of any one of SEQ ID NOs: 526-1102.

In some embodiments, the chemokine receptor antibody or immunoglobulin sequence comprises a CDRH1 comprising at least or about 70%, 80%, 85%, 90%, 91%, 92%, 93%, 94%, 95%, 96%, 97%, 98%, 99%, or 100% sequence identity to any one of SEQ ID NOs: 526-662. In some instances, the chemokine receptor antibody or immunoglobulin sequence comprises CDRH1 comprising at least or about 95% homology to any one of SEQ ID NO: 526-662. In some instances, the chemokine receptor antibody or immunoglobulin sequence comprises CDRH1 comprising at least or about 97% homology to any one of SEQ ID NO: 526-662. In some instances, the chemokine receptor antibody or immunoglobulin sequence comprises CDRH1 comprising at least or about 99% homology to any one of SEQ ID NO: 526-662. In some instances, the chemokine receptor antibody or immunoglobulin sequence comprises CDRH1 comprising 100% homology to any one of SEQ ID NO: 526-662. In some instances, the chemokine receptor antibody or immunoglobulin sequence comprises CDRH1 comprising at least a portion having at least or about 3, 4, 5, 6, 7, 8, 9, 10, 12, 14, 16, 17, 18, 19, 20, or more than 20 amino acids of any one of SEQ ID NO: 526-662.

In some embodiments, the chemokine receptor antibody or immunoglobulin sequence comprises a CDRH2 comprising at least or about 70%, 80%, 85%, 90%, 91%, 92%, 93%, 94%, 95%, 96%, 97%, 98%, 99%, or 100% sequence identity to any one of SEQ ID NOs: 663-977. In some instances, the chemokine receptor antibody or immunoglobulin sequence comprises CDRH2 comprising at least or about 95% homology to any one of SEQ ID NO: 663-977. In some instances, the chemokine receptor antibody or immunoglobulin sequence comprises CDRH2 comprising at least or about 97% homology to any one of SEQ ID NO: 663-977. In some instances, the chemokine receptor antibody or immunoglobulin sequence comprises CDRH2 comprising at least or about 99% homology to any one of SEQ ID NO: 663-977. In some instances, the chemokine receptor antibody or immunoglobulin sequence comprises CDRH2 comprising at 100% homology to any one of SEQ ID NO: 663-977. In some instances, the chemokine receptor antibody or immunoglobulin sequence comprises CDRH2 comprising at least a portion having at least or about 3, 4, 5, 6, 7, 8, 9, 10, 12, 14, 16, 17, 18, 19, 20, or more than 20 amino acids of any one of SEQ ID NO: 663-977.

In some embodiments, the chemokine receptor antibody or immunoglobulin sequence comprises a CDRH3 comprising at least or about 70%, 80%, 85%, 90%, 91%, 92%, 93%, 94%, 95%, 96%, 97%, 98%, 99%, or 100% sequence identity to any one of SEQ ID NOs: 978-1102. In some instances, the chemokine receptor antibody or immunoglobulin sequence comprises CDRH3 comprising at least or about 95% homology to any one of SEQ ID NO: 978-1102. In some instances, the chemokine receptor antibody or immunoglobulin sequence comprises CDRH3 comprising at least or about 97% homology to any one of SEQ ID NO: 978-1102. In some instances, the chemokine receptor antibody or immunoglobulin sequence comprises CDRH3 comprising at least or about 99% homology to any one of SEQ ID NO: 978-1102. In some instances, the chemokine receptor antibody or immunoglobulin sequence comprises CDRH3 comprising 100% homology to any one of SEQ ID NO: 978-1102. In some instances, the chemokine receptor antibody or immunoglobulin sequence comprises CDRH3 comprising at least a portion having at least or about 3, 4, 5, 6, 7, 8, 9, 10, 12, 14, 16, 17, 18, 19, 20, or more than 20 amino acids of any one of SEQ ID NO: 978-1102.

In some embodiments, the chemokine receptor antibody or immunoglobulin sequence comprises a CDRL1 comprising at least or about 70%, 80%, 85%, 90%, 91%, 92%, 93%, 94%, 95%, 96%, 97%, 98%, 99%, or 100% sequence identity to any one of SEQ ID NOs: 1103-1267. In some instances, the chemokine receptor antibody or immunoglobulin sequence comprises CDRL1 comprising at least or about 95% homology to any one of SEQ ID NO: 1103-1267. In some instances, the chemokine receptor antibody or immunoglobulin sequence comprises CDRL1 comprising at least or about 97% homology to any one of SEQ ID NO: 1103-1267. In some instances, the chemokine receptor antibody or immunoglobulin sequence comprises CDRL1 comprising at least or about 99% homology to any one of SEQ ID NO: 1103-1267. In some instances, the chemokine receptor antibody or immunoglobulin sequence comprises CDRL1 comprising 100% homology to any one of SEQ ID NO: 1103-1267. In some instances, the chemokine receptor antibody or immunoglobulin sequence comprises CDRL1 comprising at least a portion having at least or about 3, 4, 5, 6, 7, 8, 9, 10, 12, 14, 16, 17, 18, 19, 20, or more than 20 amino acids of any one of SEQ ID NO: 1103-1267.

In some embodiments, the chemokine receptor antibody or immunoglobulin sequence comprises a CDRL2 comprising at least or about 70%, 80%, 85%, 90%, 91%, 92%, 93%, 94%, 95%, 96%, 97%, 98%, 99%, or 100% sequence identity to any one of SEQ ID NOs: 1268-1328. In some instances, the chemokine receptor antibody or immunoglobulin sequence comprises CDRL2 comprising at least or about 95% homology to any one of SEQ ID NO: 1268-1328. In some instances, the chemokine receptor antibody or immunoglobulin sequence comprises CDRL2 comprising at least or about 97% homology to any one of SEQ ID NO: 1268-1328. In some instances, the chemokine receptor antibody or immunoglobulin sequence comprises CDRL2 comprising at least or about 99% homology to any one of SEQ ID NO: 1268-1328. In some instances, the chemokine receptor antibody or immunoglobulin sequence comprises CDRL2 comprising at 100% homology to any one of SEQ ID NO: 1268-1328. In some instances, the chemokine receptor antibody or immunoglobulin sequence comprises CDRL2 comprising at least a portion having at least or about 3, 4, 5, 6, 7, 8, 9, 10, 12, 14, 16, 17, 18, 19, 20, or more than 20 amino acids of any one of SEQ ID NO: 1268-1328.

In some embodiments, the chemokine receptor antibody or immunoglobulin sequence comprises a CDRL3 comprising at least or about 70%, 80%, 85%, 90%, 91%, 92%, 93%, 94%, 95%, 96%, 97%, 98%, 99%, or 100% sequence identity to any one of SEQ ID NOs: 1329-1493. In some instances, the chemokine receptor antibody or immunoglobulin sequence comprises CDRL3 comprising at least or about 95% homology to any one of SEQ ID NO: 1329-1493. In some instances, the chemokine receptor antibody or immunoglobulin sequence comprises CDRL3 comprising at least or about 97% homology to any one of SEQ ID NO: 1329-1493. In some instances, the chemokine receptor antibody or immunoglobulin sequence comprises CDRL3 comprising at least or about 99% homology to any one of SEQ ID NO: 1329-1493. In some instances, the chemokine receptor antibody or immunoglobulin sequence comprises CDRL3 comprising 100% homology to any one of SEQ ID NO: 1329-1493. In some instances, the chemokine receptor antibody or immunoglobulin sequence comprises CDRL3 comprising at least a portion having at least or about 3, 4, 5, 6, 7, 8, 9, 10, 12, 14, 16, 17, 18, 19, 20, or more than 20 amino acids of any one of SEQ ID NO: 1329-1493.

In some embodiments, the antibody or antibody fragment comprising a variable domain, heavy chain region (VH) and a variable domain, light chain region (VL), wherein VH comprises complementarity determining regions CDRH1, CDRH2, and CDRH3, wherein VL comprises complementarity determining regions CDRL1, CDRL2, and CDRL3, and wherein (a) an amino acid sequence of CDRH1 is as set forth in any one of SEQ ID NOs: 526-662; (b) an amino acid sequence of CDRH2 is as set forth in any one of SEQ ID NOs: 663-977; (c) an amino acid sequence of CDRH3 is as set forth in any one of SEQ ID NOs: 978-1102; (d) an amino acid sequence of CDRL1 is as set forth in any one of SEQ ID NOs: 1103-1267; (e) an amino acid sequence of CDRL2 is as set forth in any one of SEQ ID NOs: 1268-1328; and (f) an amino acid sequence of CDRL3 is as set forth in any one of SEQ ID NOs: 1329-1493. In some embodiments, the antibody or antibody fragment comprising a variable domain, heavy chain region (VH) and a variable domain, light chain region (VL), wherein VH comprises complementarity determining regions CDRH1, CDRH2, and CDRH3, wherein VL comprises complementarity determining regions CDRL1, CDRL2, and CDRL3, and wherein (a) an amino acid sequence of CDRH1 is at least or about 80%, 85%, 90%, or 95% identical to any one of SEQ ID NOs: 526-662; (b) an amino acid sequence of CDRH2 is at least or about 80%, 85%, 90%, or 95% identical to any one of SEQ ID NOs: 663-977; (c) an amino acid sequence of CDRH3 is at least or about 80%, 85%, 90%, or 95% identical to any one of SEQ ID NOs: 978-1102; (d) an amino acid sequence of CDRL1 is at least or about 80%, 85%, 90%, or 95% identical to any one of SEQ ID NOs: 1103-1267; (e) an amino acid sequence of CDRL2 is at least or about 80%, 85%, 90%, or 95% identical to any one of SEQ ID NOs: 1268-1328; and (f) an amino acid sequence of CDRL3 is at least or about 80%, 85%, 90%, or 95% identical to any one of SEQ ID NOs: 1329-1493.

In some embodiments, the antibody or antibody fragment comprising a variable domain, heavy chain region (VH) comprising complementarity determining regions CDRH1, CDRH2, and CDRH3, wherein (a) an amino acid sequence of CDRH1 is as set forth in any one of SEQ ID NOs: 526-662; (b) an amino acid sequence of CDRH2 is as set forth in any one of SEQ ID NOs: 663-977; and (c) an amino acid sequence of CDRH3 is as set forth in any one of SEQ ID NOs: 978-1102. In some embodiments, the antibody or antibody fragment comprising a variable domain, heavy chain region (VH) comprising complementarity determining regions CDRH1, CDRH2, and CDRH3, wherein (a) an amino acid sequence of CDRH1 is at least or about 80%, 85%, 90%, or 95% identical to any one of SEQ ID NOs: 526-662; (b) an amino acid sequence of CDRH2 is at least or about 80%, 85%, 90%, or 95% identical to any one of SEQ ID NOs: 663-977; and (c) an amino acid sequence of CDRH3 is at least or about 80%, 85%, 90%, or 95% identical to any one of SEQ ID NOs: 978-1102.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDRH1, CDRH2, CDRH3) and three CDRs in each light chain variable region (CDRL1, CDRL2, CDRL3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg NR and Rees AR, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Provided herein are chemokine receptor binding libraries comprising nucleic acids encoding for scaffolds comprising chemokine receptor binding domains comprise variation in domain type, domain length, or residue variation. In some instances, the domain is a region in the scaffold comprising the chemokine receptor binding domains. For example, the region is the VH, CDRH3, or VL domain. In some instances, the domain is the chemokine receptor binding domain.

Methods described herein provide for synthesis of a chemokine receptor binding library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the chemokine receptor binding library comprises varied nucleic acids collectively encoding variations at multiple positions. In some instances, the variant library comprises sequences encoding for variation of at least a single codon of a VH, CDRH3, or VL domain. In some instances, the variant library comprises sequences encoding for variation of at least a single codon in a chemokine receptor binding domain. For example, at least one single codon of a chemokine receptor binding domain as listed in Table 1 is varied. In some instances, the variant library comprises sequences encoding for variation of multiple codons of a VH, CDRH3, or VL domain. In some instances, the variant library comprises sequences encoding for variation of multiple codons in a chemokine receptor binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of a chemokine receptor binding library of nucleic acids each encoding for a predetermined variant of at least one predetermined reference nucleic acid sequence, wherein the chemokine receptor binding library comprises sequences encoding for variation of length of a domain. In some instances, the domain is VH, CDRH3, or VL domain. In some instances, the domain is the chemokine receptor binding domain. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Provided herein are chemokine receptor binding libraries comprising nucleic acids encoding for scaffolds comprising chemokine receptor binding domains, wherein the chemokine receptor binding libraries are synthesized with various numbers of fragments. In some instances, the fragments comprise the VH, CDRH3, or VL domain. In some instances, the chemokine receptor binding libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

chemokine receptor binding libraries comprising nucleic acids encoding for scaffolds comprising chemokine receptor binding domains as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 to about 75 amino acids.

chemokine receptor binding libraries comprising de novo synthesized variant sequences encoding for scaffolds comprising chemokine receptor binding domains comprise a number of variant sequences. In some instances, a number of variant sequences is de novo synthesized for a CDRH1, CDRH2, CDRH3, CDRL1, CDRL2, CDRL3, VL, VH, or a combination thereof. In some instances, a number of variant sequences is de novo synthesized for framework element 1 (FW1), framework element 2 (FW2), framework element 3 (FW3), or framework element 4 (FW4). In some instances, a number of variant sequences is de novo synthesized for a GPCR binding domain. For example, the number of variant sequences is about 1 to about 10 sequences for the VH domain, about 10⁸ sequences for the chemokine receptor binding domain, and about 1 to about 44 sequences for the VK domain. The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is about 10 to 300, 25 to 275, 50 to 250, 75 to 225, 100 to 200, or 125 to 150 sequences.

chemokine receptor binding libraries comprising de novo synthesized variant sequences encoding for scaffolds comprising chemokine receptor binding domains comprise improved diversity. For example, variants are generated by placing chemokine receptor binding domain variants in immunoglobulin scaffold variants comprising N-terminal CDRH3 variations and C-terminal CDRH3 variations. In some instances, variants include affinity maturation variants. Alternatively or in combination, variants include variants in other regions of the immunoglobulin including, but not limited to, CDRH1, CDRH2, CDRL1, CDRL2, and CDRL3. In some instances, the number of variants of the chemokine receptor binding libraries is least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ non-identical sequences. For example, a library comprising about 10 variant sequences for a VH region, about 237 variant sequences for a CDRH3 region, and about 43 variant sequences for a VL and CDRL3 region comprises 10⁵ non-identical sequences (10×237×43).

Provided herein are libraries comprising nucleic acids encoding for a chemokine receptor antibody comprising variation in at least one region of the antibody, wherein the region is the CDR region. In some instances, the chemokine receptor antibody is a single domain antibody comprising one heavy chain variable domain such as a VHH antibody. In some instances, the VHH antibody comprises variation in one or more CDR regions. In some instances, libraries described herein comprise at least or about 1, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3. For example, the libraries comprise at least 2000 sequences of a CDR1, at least 1200 sequences for CDR2, and at least 1600 sequences for CDR3. In some instances, each sequence is non-identical.

In some instances, the CDR1, CDR2, or CDR3 is of a variable domain, light chain (VL). CDR1, CDR2, or CDR3 of a variable domain, light chain (VL) can be referred to as CDRL1, CDRL2, or CDRL3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VL. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VL. For example, the libraries comprise at least 20 sequences of a CDR1 of the VL, at least 4 sequences of a CDR2 of the VL, and at least 140 sequences of a CDR3 of the VL. In some instances, the libraries comprise at least 2 sequences of a CDR1 of the VL, at least 1 sequence of CDR2 of the VL, and at least 3000 sequences of a CDR3 of the VL. In some instances, the VL is IGKV1-39, IGKV1-9, IGKV2-28, IGKV3-11, IGKV3-15, IGKV3-20, IGKV4-1, IGLV1-51, IGLV2-14, IGLV1-40, or IGLV3-1. In some instances, the VL is IGKV2-28. In some instances, the VL is IGLV1-51.

In some instances, the CDR1, CDR2, or CDR3 is of a variable domain, heavy chain (VH). CDR1, CDR2, or CDR3 of a variable domain, heavy chain (VH) can be referred to as CDRH1, CDRH2, or CDRH3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VH. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VH. For example, the libraries comprise at least 30 sequences of a CDR1 of the VH, at least 570 sequences of a CDR2 of the VH, and at least 10⁸ sequences of a CDR3 of the VH. In some instances, the libraries comprise at least 30 sequences of a CDR1 of the VH, at least 860 sequences of a CDR2 of the VH, and at least 10⁷ sequences of a CDR3 of the VH. In some instances, the VH is IGHV1-18, IGHV1-69, IGHV1-8 IGHV3-21, IGHV3-23, IGHV3-30/33m, IGHV3-28, IGHV3-74, IGHV4-39, or IGHV4-59/61. In some instances, the VH is IGHV1-69, IGHV3-30, IGHV3-23, IGHV3, IGHV1-46, IGHV3-7, IGHV1, or IGHV1-8. In some instances, the VH is IGHV1-69 or IGHV3-30. In some instances, the VH is IGHV3-23.

Libraries as described herein, in some embodiments, comprise varying lengths of a CDRL1, CDRL2, CDRL3, CDRH1, CDRH2, or CDRH3. In some instances, the length of the CDRL1, CDRL2, CDRL3, CDRH1, CDRH2, or CDRH3 comprises at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length. For example, the CDRH3 comprises at least or about 12, 15, 16, 17, 20, 21, or 23 amino acids in length. In some instances, the CDRL1, CDRL2, CDRL3, CDRH1, CDRH2, or CDRH3 comprises a range of about 1 to about 10, about 5 to about 15, about 10 to about 20, or about 15 to about 30 amino acids in length.

Libraries comprising nucleic acids encoding for antibodies having variant CDR sequences as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the antibodies comprise at least or about 100, 200, 300, 400, 500, 600, 700, 800, 900, 1000, 2000, 3000, 4000, 5000, or more than 5000 amino acids.

Ratios of the lengths of a CDRL1, CDRL2, CDRL3, CDRH1, CDRH2, or CDRH3 may vary in libraries described herein. In some instances, a CDRL1, CDRL2, CDRL3, CDRH1, CDRH2, or CDRH3 comprising at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length comprises about 10%, 20%, 30%, 40%, 50%, 60%, 70%, 80%, 90%, or more than 90% of the library. For example, a CDRH3 comprising about 23 amino acids in length is present in the library at 40%, a CDRH3 comprising about 21 amino acids in length is present in the library at 30%, a CDRH3 comprising about 17 amino acids in length is present in the library at 20%, and a CDRH3 comprising about 12 amino acids in length is present in the library at 10%. In some instances, a CDRH3 comprising about 20 amino acids in length is present in the library at 40%, a CDRH3 comprising about 16 amino acids in length is present in the library at 30%, a CDRH3 comprising about 15 amino acids in length is present in the library at 20%, and a CDRH3 comprising about 12 amino acids in length is present in the library at 10%.

Libraries as described herein encoding for a VHH antibody comprise variant CDR sequences that are shuffled to generate a library with a theoretical diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences. In some instances, the library has a final library diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences.

Provided herein are chemokine receptor binding libraries encoding for an immunoglobulin. In some instances, the chemokine receptor immunoglobulin is an antibody. In some instances, the chemokine receptor immunoglobulin is a VHH antibody. In some instances, the chemokine receptor immunoglobulin comprises a binding affinity (e.g., K_{D}) to chemokine receptor of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the chemokine receptor immunoglobulin comprises a K_{D} of less than 1 nM. In some instances, the chemokine receptor immunoglobulin comprises a K_{D} of less than 1.2 nM. In some instances, the chemokine receptor immunoglobulin comprises a K_{D} of less than 2 nM. In some instances, the chemokine receptor immunoglobulin comprises a K_{D} of less than 5 nM. In some instances, the chemokine receptor immunoglobulin comprises a K_{D} of less than 10 nM. In some instances, the chemokine receptor immunoglobulin comprises a K_{D} of less than 13.5 nM. In some instances, the chemokine receptor immunoglobulin comprises a K_{D} of less than 15 nM. In some instances, the chemokine receptor immunoglobulin comprises a K_{D} of less than 20 nM. In some instances, the chemokine receptor immunoglobulin comprises a K_{D} of less than 25 nM. In some instances, the chemokine receptor immunoglobulin comprises a K_{D} of less than 30 nM.

In some instances, the chemokine receptor immunoglobulin is a chemokine receptor agonist. In some instances, the chemokine receptor immunoglobulin is a chemokine receptor antagonist. In some instances, the chemokine receptor immunoglobulin is a chemokine receptor allosteric modulator. In some instances, the allosteric modulator is a negative allosteric modulator. In some instances, the allosteric modulator is a positive allosteric modulator. In some instances, the chemokine receptor immunoglobulin results in agonistic, antagonistic, or allosteric effects at a concentration of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, 1000 nM, or more than 1000 nM. In some instances, the chemokine receptor immunoglobulin is a negative allosteric modulator. In some instances, the chemokine receptor immunoglobulin is a negative allosteric modulator at a concentration of at least or about 0.001, 0.005, 0.01, 0.05, 0.1, 0.5, 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM. In some instances, the chemokine receptor immunoglobulin is a negative allosteric modulator at a concentration in a range of about 0.001 to about 100, 0.01 to about 90, about 0.1 to about 80, 1 to about 50, about 10 to about 40 nM, or about 1 to about 10 nM. In some instances, the chemokine receptor immunoglobulin comprises an EC50 or IC50 of at least or about 0.001, 0.0025, 0.005, 0.01, 0.025, 0.05, 0.06, 0.07, 0.08, 0.9, 0.1, 0.5, 1, 2, 3, 4, 5, 6, or more than 6 nM. In some instances, the chemokine receptor immunoglobulin comprises an EC50 or IC50 of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM.

Provided herein are chemokine receptor binding libraries encoding for an immunoglobulin, wherein the immunoglobulin comprises a long half-life. In some instances, the half-life of the chemokine receptor immunoglobulin is at least or about 12 hours, 24 hours 36 hours, 48 hours, 60 hours, 72 hours, 84 hours, 96 hours, 108 hours, 120 hours, 140 hours, 160 hours, 180 hours, 200 hours, or more than 200 hours. In some instances, the half-life of the chemokine receptor immunoglobulin is in a range of about 12 hours to about 300 hours, about 20 hours to about 280 hours, about 40 hours to about 240 hours, or about 60 hours to about 200 hours.

chemokine receptor immunoglobulins as described herein may comprise improved properties. In some instances, the chemokine receptor immunoglobulins are monomeric. In some instances, the chemokine receptor immunoglobulins are not prone to aggregation. In some instances, at least or about 70%, 75%, 80%, 85%, 90%, 95%, or 99% of the chemokine receptor immunoglobulins are monomeric. In some instances, the chemokine receptor immunoglobulins are thermostable. In some instances, the chemokine receptor immunoglobulins result in reduced non-specific binding.

Following synthesis of chemokine receptor binding libraries comprising nucleic acids encoding scaffolds comprising chemokine receptor binding domains, libraries may be used for screening and analysis. For example, libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, the chemokine receptor binding libraries comprises nucleic acids encoding scaffolds comprising GPCR binding domains with multiple tags such as GFP, FLAG, and Lucy as well as a DNA barcode. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

Expression Systems

Provided herein are libraries comprising nucleic acids encoding for scaffolds comprising chemokine receptor binding domains, wherein the libraries have improved specificity, stability, expression, folding, or downstream activity. In some instances, libraries described herein are used for screening and analysis.

Provided herein are libraries comprising nucleic acids encoding for scaffolds comprising chemokine receptor binding domains, wherein the nucleic acid libraries are used for screening and analysis. In some instances, screening and analysis comprises in vitro, in vivo, or ex vivo assays. Cells for screening include primary cells taken from living subjects or cell lines. Cells may be from prokaryotes (e.g., bacteria and fungi) or eukaryotes (e.g., animals and plants). Exemplary animal cells include, without limitation, those from a mouse, rabbit, primate, and insect. In some instances, cells for screening include a cell line including, but not limited to, Chinese Hamster Ovary (CHO) cell line, human embryonic kidney (HEK) cell line, or baby hamster kidney (BHK) cell line. In some instances, nucleic acid libraries described herein may also be delivered to a multicellular organism. Exemplary multicellular organisms include, without limitation, a plant, a mouse, rabbit, primate, and insect.

Nucleic acid libraries or protein libraries encoded thereof described herein may be screened for various pharmacological or pharmacokinetic properties. In some instances, the libraries are screened using in vitro assays, in vivo assays, or ex vivo assays. For example, in vitro pharmacological or pharmacokinetic properties that are screened include, but are not limited to, binding affinity, binding specificity, and binding avidity. Exemplary in vivo pharmacological or pharmacokinetic properties of libraries described herein that are screened include, but are not limited to, therapeutic efficacy, activity, preclinical toxicity properties, clinical efficacy properties, clinical toxicity properties, immunogenicity, potency, and clinical safety properties.

Pharmacological or pharmacokinetic properties that may be screened include, but are not limited to, cell binding affinity and cell activity. For example, cell binding affinity assays or cell activity assays are performed to determine agonistic, antagonistic, or allosteric effects of libraries described herein. In some instances, the cell activity assay is a cAMP assay. In some instances, libraries as described herein are compared to cell binding or cell activity of ligands of chemokine receptor.

Libraries as described herein may be screened in cell based assays or in non-cell based assays. Examples of non-cell based assays include, but are not limited to, using viral particles, using in vitro translation proteins, and using protealiposomes with chemokine receptor.

Nucleic acid libraries as described herein may be screened by sequencing. In some instances, next generation sequence is used to determine sequence enrichment of chemokine receptor binding variants. In some instances, V gene distribution, J gene distribution, V gene family, CDR3 counts per length, or a combination thereof is determined. In some instances, clonal frequency, clonal accumulation, lineage accumulation, or a combination thereof is determined. In some instances, number of sequences, sequences with VH clones, clones, clones greater than 1, clonotypes, clonotypes greater than 1, lineages, simpsons, or a combination thereof is determined. In some instances, a percentage of non-identical CDR3s is determined. For example, the percentage of non-identical CDR3s is calculated as the number of non-identical CDR3s in a sample divided by the total number of sequences that had a CDR3 in the sample.

Provided herein are nucleic acid libraries, wherein the nucleic acid libraries may be expressed in a vector. Expression vectors for inserting nucleic acid libraries disclosed herein may comprise eukaryotic or prokaryotic expression vectors. Exemplary expression vectors include, without limitation, mammalian expression vectors: pSF-CMV-NEO-NH2-PPT-3XFLAG, pSF-CMV-NEO—COOH-3XFLAG, pSF-CMV—PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag(m), and pSF-CMV—PURO-NH2-CMYC; bacterial expression vectors: pSF-OXB20-BetaGal,pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8. In some instances, the vector is pcDNA3 or pcDNA3.1.

Described herein are nucleic acid libraries that are expressed in a vector to generate a construct comprising a scaffold comprising sequences of chemokine receptor binding domains. In some instances, a size of the construct varies. In some instances, the construct comprises at least or about 500, 600, 700, 800, 900, 1000, 1100, 1300, 1400, 1500, 1600, 1700, 1800, 2000, 2400, 2600, 2800, 3000, 3200, 3400, 3600, 3800, 4000, 4200, 4400, 4600, 4800, 5000, 6000, 7000, 8000, 9000, 10000, or more than 10000 bases. In some instances, a the construct comprises a range of about 300 to 1,000, 300 to 2,000, 300 to 3,000, 300 to 4,000, 300 to 5,000, 300 to 6,000, 300 to 7,000, 300 to 8,000, 300 to 9,000, 300 to 10,000, 1,000 to 2,000, 1,000 to 3,000, 1,000 to 4,000, 1,000 to 5,000, 1,000 to 6,000, 1,000 to 7,000, 1,000 to 8,000, 1,000 to 9,000, 1,000 to 10,000, 2,000 to 3,000, 2,000 to 4,000, 2,000 to 5,000, 2,000 to 6,000, 2,000 to 7,000, 2,000 to 8,000, 2,000 to 9,000, 2,000 to 10,000, 3,000 to 4,000, 3,000 to 5,000, 3,000 to 6,000, 3,000 to 7,000, 3,000 to 8,000, 3,000 to 9,000, 3,000 to 10,000, 4,000 to 5,000, 4,000 to 6,000, 4,000 to 7,000, 4,000 to 8,000, 4,000 to 9,000, 4,000 to 10,000, 5,000 to 6,000, 5,000 to 7,000, 5,000 to 8,000, 5,000 to 9,000, 5,000 to 10,000, 6,000 to 7,000, 6,000 to 8,000, 6,000 to 9,000, 6,000 to 10,000, 7,000 to 8,000, 7,000 to 9,000, 7,000 to 10,000, 8,000 to 9,000, 8,000 to 10,000, or 9,000 to 10,000 bases.

Provided herein are libraries comprising nucleic acids encoding for scaffolds comprising GPCR binding domains, wherein the nucleic acid libraries are expressed in a cell. In some instances, the libraries are synthesized to express a reporter gene. Exemplary reporter genes include, but are not limited to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination.

Diseases and Disorders

Provided herein are chemokine receptor binding libraries comprising nucleic acids encoding for scaffolds comprising chemokine receptor binding domains that may have therapeutic effects. In some instances, the chemokine receptor binding libraries result in protein when translated that is used to treat a disease or disorder. In some instances, the protein is an immunoglobulin. In some instances, the protein is a peptidomimetic.

Chemokine receptor libraries as described herein may comprise modulators of chemokine receptor. In some instances, the chemokine receptor modulator is an inhibitor. In some instances, the chemokine receptor modulator is an activator. In some instances, the chemokine receptor inhibitor is a chemokine receptor antagonist. Modulators of chemokine receptors, in some instances, are used for treating various diseases or disorders.

Exemplary diseases include, but are not limited to, cancer, inflammatory diseases or disorders, a metabolic disease or disorder, a cardiovascular disease or disorder, a respiratory disease or disorder, pain, a digestive disease or disorder, a reproductive disease or disorder, an endocrine disease or disorder, or a neurological disease or disorder. In some instances, the cancer is a solid cancer or a hematologic cancer. In some instances, the cancer is gastric cancer, breast cancer, colorectal cancer, lung cancer, prostate cancer, hepatocellular carcinoma, leukemia, or lymphoma. In some instances, the cancer is B-cell non-Hodgkin lymphoma. In some instances, the disease or disorder is caused by a virus. In some instances, the disease or disorder is caused by human immunodeficiency virus (HIV).

In some instances, the chemokine receptor modulator is involved in immune surveillance. In some instances, the chemokine receptor modulator is involved in T cell entry by a virus. In some instances, the chemokine receptor modulator is involved in diseases or disorders affecting homeostasis. In some instances, the chemokine receptor modulator is involved in disease or disorders relating to hematopoietic stem cell migration.

Described herein, in some embodiments, are antibodies or antibody fragment thereof that binds chemokine receptor for use in diagnosing or establishing a disease or disorder in a subject. In some embodiments, the antibody or antibody fragment thereof comprises a sequence as set forth in any one of SEQ ID NOs: 7-1493. In some embodiments, the antibodies or antibody fragment is used for diagnosing or establishing cancer, inflammatory diseases or disorders, a metabolic disease or disorder, a cardiovascular disease or disorder, a respiratory disease or disorder, pain, a digestive disease or disorder, a reproductive disease or disorder, an endocrine disease or disorder, or a neurological disease or disorder in a subject. In some embodiments, the antibodies or antibody fragment is used for diagnosing or establishing solid cancer or a hematologic cancer. In some embodiments, the antibodies or antibody fragment is used for diagnosing or establishing gastric cancer, breast cancer, colorectal cancer, lung cancer, prostate cancer, hepatocellular carcinoma, leukemia, or lymphoma. In some embodiments, the antibodies or antibody fragment is used for diagnosing or establishing B-cell non-Hodgkin lymphoma. In some embodiments, the antibodies or antibody fragment is used for diagnosing or establishing a viral infection (e.g., caused by HIV).

In some instances, the subject is a mammal. In some instances, the subject is a mouse, rabbit, dog, or human. Subjects treated by methods described herein may be infants, adults, or children. Pharmaceutical compositions comprising antibodies or antibody fragments as described herein may be administered intravenously or subcutaneously.

Described herein are pharmaceutical compositions comprising antibodies or antibody fragment thereof that binds chemokine receptor. In some embodiments, the antibody or antibody fragment thereof comprises a sequence as set forth in any one of SEQ ID NOs: 7-1493. In some embodiments, the antibody or antibody fragment thereof comprises a sequence that is at least or about 70%, 80%, 85%, 90%, 91%, 92%, 93%, 94%, 95%, 96%, 97%, 98%, 99%, or 100% sequence identity to a sequence as set forth in any one of SEQ ID NOs: 7-1493.

Described herein are pharmaceutical compositions comprising antibodies or antibody fragment thereof that binds chemokine receptor that comprise various dosages of the antibodies or antibody fragment. In some instances, the dosage is ranging from about 1 to 80 mg/kg, from about 1 to about 100 mg/kg, from about 5 to about 100 mg/kg, from about 5 to about 80 mg/kg, from about 5 to about 60 mg/kg, from about 5 to about 50 mg/kg or from about 5 to about 500 mg/kg which can be administered in single or multiple doses. In some instances, the dosage is administered in an amount of about 0.01 mg/kg, about 0.05 mg/kg, about 0.10 mg/kg, about 0.25 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 240, about 250, about 260, about 270, about 275, about 280, about 290, about 300, about 310, about 320, about 330, about 340, about 350, about 360 mg/kg, about 370 mg/kg, about 380 mg/kg, about 390 mg/kg, about 400 mg/kg, 410 mg/kg, about 420 mg/kg, about 430 mg/kg, about 440 mg/kg, about 450 mg/kg, about 460 mg/kg, about 470 mg/kg, about 480 mg/kg, about 490 mg/kg, or about 500 mg/kg.

Variant Libraries

Codon Variation

Variant nucleic acid libraries described herein may comprise a plurality of nucleic acids, wherein each nucleic acid encodes for a variant codon sequence compared to a reference nucleic acid sequence. In some instances, each nucleic acid of a first nucleic acid population contains a variant at a single variant site. In some instances, the first nucleic acid population contains a plurality of variants at a single variant site such that the first nucleic acid population contains more than one variant at the same variant site. The first nucleic acid population may comprise nucleic acids collectively encoding multiple codon variants at the same variant site. The first nucleic acid population may comprise nucleic acids collectively encoding up to 19 or more codons at the same position. The first nucleic acid population may comprise nucleic acids collectively encoding up to 60 variant triplets at the same position, or the first nucleic acid population may comprise nucleic acids collectively encoding up to 61 different triplets of codons at the same position. Each variant may encode for a codon that results in a different amino acid during translation. Table 3 provides a listing of each codon possible (and the representative amino acid) for a variant site.

TABLE 2
List of codons and amino acids

| | One | Three | |
|---|---|---|---|
| | letter | letter | |
| Amino Acids | code | code | Codons |
| Alanine | A | Ala | GCA GCC GCG GCT |
| Cysteine | C | Cys | TGC TGT |
| Aspartic acid | D | Asp | GAC GAT |
| Glutamic acid | E | Glu | GAA GAG |
| Phenylalanine | F | Phe | TTC TTT |
| Glycine | G | Gly | GGA GGC GGG GGT |
| Histidine | H | His | CAC CAT |
| Isoleucine | I | Iso | ATA ATC ATT |
| Lysine | K | Lys | AAA AAG |
| Leucine | L | Leu | TTA TTG CTA CTC CTG |
| | | | CTT |
| Methionine | M | Met | ATG |
| Asparagine | N | Asn | AAC AAT |
| Proline | P | Pro | CCA CCC CCG CCT |
| Glutamine | Q | Gln | CAA CAG |
| Arginine | R | Arg | AGA AGG CGA CGC CGG |
| | | | CGT |
| Serine | S | Ser | AGC AGT TCA TCC TCG |
| | | | TCT |
| Threonine | T | Thr | ACA ACC ACG ACT |
| Valine | V | Val | GTA GTC GTG GTT |
| Tryptophan | W | Trp | TGG |
| Tyrosine | Y | Tyr | TAC TAT |

A nucleic acid population may comprise varied nucleic acids collectively encoding up to 20 codon variations at multiple positions. In such cases, each nucleic acid in the population comprises variation for codons at more than one position in the same nucleic acid. In some instances, each nucleic acid in the population comprises variation for codons at 1, 2, 3, 4, 5, 6, 7, 8, 9, 10, 11, 12, 13, 14, 15, 16, 17, 18, 19, 20 or more codons in a single nucleic acid. In some instances, each variant long nucleic acid comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single long nucleic acid. In some instances, the variant nucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single nucleic acid. In some instances, the variant nucleic acid population comprises variation for codons in at least about 10, 20, 30, 40, 50, 60, 70, 80, 90, 100 or more codons in a single long nucleic acid.

Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform is capable of increasing throughput by a factor of up to 1,000 or more compared to traditional synthesis methods, with production of up to approximately 1,000,000 or more polynucleotides, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene mutants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations at a specific site within the receptor, represents one approach to this development challenge. Though costly and time and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing—in other words, a precision library, enables reduced costs as well as turnaround time for screening. Provided herein are methods for synthesizing nucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, a drug itself can be optimized using methods described herein. For example, to improve a specified function of an antibody, a variant polynucleotide library encoding for a portion of the antibody is designed and synthesized. A variant nucleic acid library for the antibody can then be generated by processes described herein (e.g., PCR mutagenesis followed by insertion into a vector). The antibody is then expressed in a production cell line and screened for enhanced activity. Example screens include examining modulation in binding affinity to an antigen, stability, or effector function (e.g., ADCC, complement, or apoptosis). Exemplary regions to optimize the antibody include, without limitation, the Fc region, Fab region, variable region of the Fab region, constant region of the Fab region, variable domain of the heavy chain or light chain (V_{H} or V_{L}), and specific complementarity-determining regions (CDRs) of V_{H} or V_{L}.

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

To identify a variant molecule associated with prevention, reduction or treatment of a disease state, a variant nucleic acid library described herein is expressed in a cell associated with a disease state, or one in which a cell a disease state can be induced. In some instances, an agent is used to induce a disease state in cells. Exemplary tools for disease state induction include, without limitation, a Cre/Lox recombination system, LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof

Substrates

Devices used as a surface for polynucleotide synthesis may be in the form of substrates which include, without limitation, homogenous array surfaces, patterned array surfaces, channels, beads, gels, and the like. Provided herein are substrates comprising a plurality of clusters, wherein each cluster comprises a plurality of loci that support the attachment and synthesis of polynucleotides. In some instances, substrates comprise a homogenous array surface. For example, the homogenous array surface is a homogenous plate. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus is on a three-dimensional surface, e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some cases, a surface of a substrate is inclusive of one or a plurality of surfaces of a substrate. The average error rates for polynucleotides synthesized within a library described here using the systems and methods provided are often less than 1 in 1000, less than about 1 in 2000, less than about 1 in 3000 or less often without error correction.

Provided herein are surfaces that support the parallel synthesis of a plurality of polynucleotides having different predetermined sequences at addressable locations on a common support. In some instances, a substrate provides support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the surfaces provide support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the substrate provides a surface environment for the growth of polynucleotides having at least 80, 90, 100, 120, 150, 175, 200, 225, 250, 275, 300, 325, 350, 375, 400, 425, 450, 475, 500 bases or more.

Provided herein are methods for polynucleotide synthesis on distinct loci of a substrate, wherein each locus supports the synthesis of a population of polynucleotides. In some cases, each locus supports the synthesis of a population of polynucleotides having a different sequence than a population of polynucleotides grown on another locus. In some instances, each polynucleotide sequence is synthesized with 1, 2, 3, 4, 5, 6, 7, 8, 9 or more redundancy across different loci within the same cluster of loci on a surface for polynucleotide synthesis. In some instances, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a substrate comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a substrate comprises about 10,000 distinct loci. The amount of loci within a single cluster is varied in different instances. In some cases, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci. Alternatively or in combination, polynucleotide synthesis occurs on a homogenous array surface.

In some instances, the number of distinct polynucleotides synthesized on a substrate is dependent on the number of distinct loci available in the substrate. In some instances, the density of loci within a cluster or surface of a substrate is at least or about 1, 10, 25, 50, 65, 75, 100, 130, 150, 175, 200, 300, 400, 500, 1,000 or more loci per mm². In some cases, a substrate comprises 10-500, 25-400, 50-500, 100-500, 150-500, 10-250, 50-250, 10-200, or 50-200 mm². In some instances, the distance between the centers of two adjacent loci within a cluster or surface is from about 10-500, from about 10-200, or from about 10-100 um. In some instances, the distance between two centers of adjacent loci is greater than about 10, 20, 30, 40, 50, 60, 70, 80, 90 or 100 um. In some instances, the distance between the centers of two adjacent loci is less than about 200, 150, 100, 80, 70, 60, 50, 40, 30, 20 or 10 um. In some instances, each locus has a width of about 0.5, 1, 2, 3, 4, 5, 6, 7, 8, 9, 10, 20, 30, 40, 50, 60, 70, 80, 90 or 100 um. In some cases, each locus has a width of about 0.5-100, 0.5-50, 10-75, or 0.5-50 um.

In some instances, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a substrate comprises from about 1 cluster per 10 mm² to about 10 clusters per 1 mm². In some instances, the distance between the centers of two adjacent clusters is at least or about 50, 100, 200, 500, 1000, 2000, or 5000 um. In some cases, the distance between the centers of two adjacent clusters is between about 50-100, 50-200, 50-300, 50-500, and 100-2000 um. In some cases, the distance between the centers of two adjacent clusters is between about 0.05-50, 0.05-10, 0.05-5, 0.05-4, 0.05-3, 0.05-2, 0.1-10, 0.2-10, 0.3-10, 0.4-10, 0.5-10, 0.5-5, or 0.5-2 mm. In some cases, each cluster has a cross section of about 0.5 to about 2, about 0.5 to about 1, or about 1 to about 2 mm. In some cases, each cluster has a cross section of about 0.5, 0.6, 0.7, 0.8, 0.9, 1, 1.1, 1.2, 1.3, 1.4, 1.5, 1.6, 1.7, 1.8, 1.9 or 2 mm. In some cases, each cluster has an interior cross section of about 0.5, 0.6, 0.7, 0.8, 0.9, 1, 1.1, 1.15, 1.2, 1.3, 1.4, 1.5, 1.6, 1.7, 1.8, 1.9 or 2 mm.

In some instances, a substrate is about the size of a standard 96 well plate, for example between about 100 and about 200 mm by between about 50 and about 150 mm. In some instances, a substrate has a diameter less than or equal to about 1000, 500, 450, 400, 300, 250, 200, 150, 100 or 50 mm. In some instances, the diameter of a substrate is between about 25-1000, 25-800, 25-600, 25-500, 25-400, 25-300, or 25-200 mm. In some instances, a substrate has a planar surface area of at least about 100; 200; 500; 1,000; 2,000; 5,000; 10,000; 12,000; 15,000; 20,000; 30,000; 40,000; 50,000 mm² or more. In some instances, the thickness of a substrate is between about 50-2000, 50-1000, 100-1000, 200-1000, or 250-1000 mm.

Surface Materials

Substrates, devices, and reactors provided herein are fabricated from any variety of materials suitable for the methods, compositions, and systems described herein. In certain instances, substrate materials are fabricated to exhibit a low level of nucleotide binding. In some instances, substrate materials are modified to generate distinct surfaces that exhibit a high level of nucleotide binding. In some instances, substrate materials are transparent to visible and/or UV light. In some instances, substrate materials are sufficiently conductive, e.g., are able to form uniform electric fields across all or a portion of a substrate. In some instances, conductive materials are connected to an electric ground. In some instances, the substrate is heat conductive or insulated. In some instances, the materials are chemical resistant and heat resistant to support chemical or biochemical reactions, for example polynucleotide synthesis reaction processes. In some instances, a substrate comprises flexible materials. For flexible materials, materials can include, without limitation: nylon, both modified and unmodified, nitrocellulose, polypropylene, and the like. In some instances, a substrate comprises rigid materials. For rigid materials, materials can include, without limitation: glass; fuse silica; silicon, plastics (for example polytetraflouroethylene, polypropylene, polystyrene, polycarbonate, and blends thereof, and the like); metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

Surface Architecture

Provided herein are substrates for the methods, compositions, and systems described herein, wherein the substrates have a surface architecture suitable for the methods, compositions, and systems described herein. In some instances, a substrate comprises raised and/or lowered features. One benefit of having such features is an increase in surface area to support polynucleotide synthesis. In some instances, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as a material deposition device. In some cases, reagents and/or fluids collect in a larger well in fluid communication one or more channels. For example, a substrate comprises a plurality of channels corresponding to a plurality of loci with a cluster, and the plurality of channels are in fluid communication with one well of the cluster. In some methods, a library of polynucleotides is synthesized in a plurality of loci of a cluster.

Provided herein are substrates for the methods, compositions, and systems described herein, wherein the substrates are configured for polynucleotide synthesis. In some instances, the structure is configured to allow for controlled flow and mass transfer paths for polynucleotide synthesis on a surface. In some instances, the configuration of a substrate allows for the controlled and even distribution of mass transfer paths, chemical exposure times, and/or wash efficacy during polynucleotide synthesis. In some instances, the configuration of a substrate allows for increased sweep efficiency, for example by providing sufficient volume for a growing polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are substrates for the methods, compositions, and systems described herein, wherein the substrates comprise structures suitable for the methods, compositions, and systems described herein. In some instances, segregation is achieved by physical structure. In some instances, segregation is achieved by differential functionalization of the surface generating active and passive regions for polynucleotide synthesis. In some instances, differential functionalization is achieved by alternating the hydrophobicity across the substrate surface, thereby creating water contact angle effects that cause beading or wetting of the deposited reagents. Employing larger structures can decrease splashing and cross-contamination of distinct polynucleotide synthesis locations with reagents of the neighboring spots. In some cases, a device, such as a material deposition device, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000, 1:3,000, 1:5,000, or 1:10,000). In some cases, a substrate comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a substrate may have the same or different width, height, and/or volume as another well of the substrate. A channel of a substrate may have the same or different width, height, and/or volume as another channel of the substrate. In some instances, the diameter of a cluster or the diameter of a well comprising a cluster, or both, is between about 0.05-50, 0.05-10, 0.05-5, 0.05-4, 0.05-3, 0.05-2, 0.05-1, 0.05-0.5, 0.05-0.1, 0.1-10, 0.2-10, 0.3-10, 0.4-10, 0.5-10, 0.5-5, or 0.5-2 mm. In some instances, the diameter of a cluster or well or both is less than or about 5, 4, 3, 2, 1, 0.5, 0.1, 0.09, 0.08, 0.07, 0.06, or 0.05 mm. In some instances, the diameter of a cluster or well or both is between about 1.0 and 1.3 mm. In some instances, the diameter of a cluster or well, or both is about 1.150 mm. In some instances, the diameter of a cluster or well, or both is about 0.08 mm. The diameter of a cluster refers to clusters within a two-dimensional or three-dimensional substrate.

In some instances, the height of a well is from about 20-1000, 50-1000, 100-1000, 200-1000, 300-1000, 400-1000, or 500-1000 um. In some cases, the height of a well is less than about 1000, 900, 800, 700, or 600 um.

In some instances, a substrate comprises a plurality of channels corresponding to a plurality of loci within a cluster, wherein the height or depth of a channel is 5-500, 5-400, 5-300, 5-200, 5-100, 5-50, or 10-50 um. In some cases, the height of a channel is less than 100, 80, 60, 40, or 20 um.

In some instances, the diameter of a channel, locus (e.g., in a substantially planar substrate) or both channel and locus (e.g., in a three-dimensional substrate wherein a locus corresponds to a channel) is from about 1-1000, 1-500, 1-200, 1-100, 5-100, or 10-100 um, for example, about 90, 80, 70, 60, 50, 40, 30, 20 or 10 um. In some instances, the diameter of a channel, locus, or both channel and locus is less than about 100, 90, 80, 70, 60, 50, 40, 30, 20 or 10 um. In some instances, the distance between the center of two adjacent channels, loci, or channels and loci is from about 1-500, 1-200, 1-100, 5-200, 5-100, 5-50, or 5-30, for example, about 20 um.

Surface Modifications

Provided herein are methods for polynucleotide synthesis on a surface, wherein the surface comprises various surface modifications. In some instances, the surface modifications are employed for the chemical and/or physical alteration of a surface by an additive or subtractive process to change one or more chemical and/or physical properties of a substrate surface or a selected site or region of a substrate surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some cases, the adhesion promoter is a chemical with a high surface energy. In some instances, a second chemical layer is deposited on a surface of a substrate. In some cases, the second chemical layer has a low surface energy. In some cases, surface energy of a chemical layer coated on a surface supports localization of droplets on the surface. Depending on the patterning arrangement selected, the proximity of loci and/or area of fluid contact at the loci are alterable.

In some instances, a substrate surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a substrate surface is modified with one or more different layers of compounds. Such modification layers of interest include, without limitation, inorganic and organic layers such as metals, metal oxides, polymers, small organic molecules and the like.

In some instances, resolved loci of a substrate are functionalized with one or more moieties that increase and/or decrease surface energy. In some cases, a moiety is chemically inert. In some cases, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for substrate functionalization comprises: (a) providing a substrate having a surface that comprises silicon dioxide; and (b) silanizing the surface using, a suitable silanizing agent described herein or otherwise known in the art, for example, an organofunctional alkoxysilane molecule. Methods and functionalizing agents are described in U.S. Pat. No. 5,474,796, which is herein incorporated by reference in its entirety.

In some instances, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally covers a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions.

Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 min, 1 min, 50 sec, 40 sec, 30 sec, 20 sec and 10 sec.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′-OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example, at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150,22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides is synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may not be adjacent and separated by 1, 2, 3, 4, 5, 6, 7, 8, 9, 10, or more codons.

In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

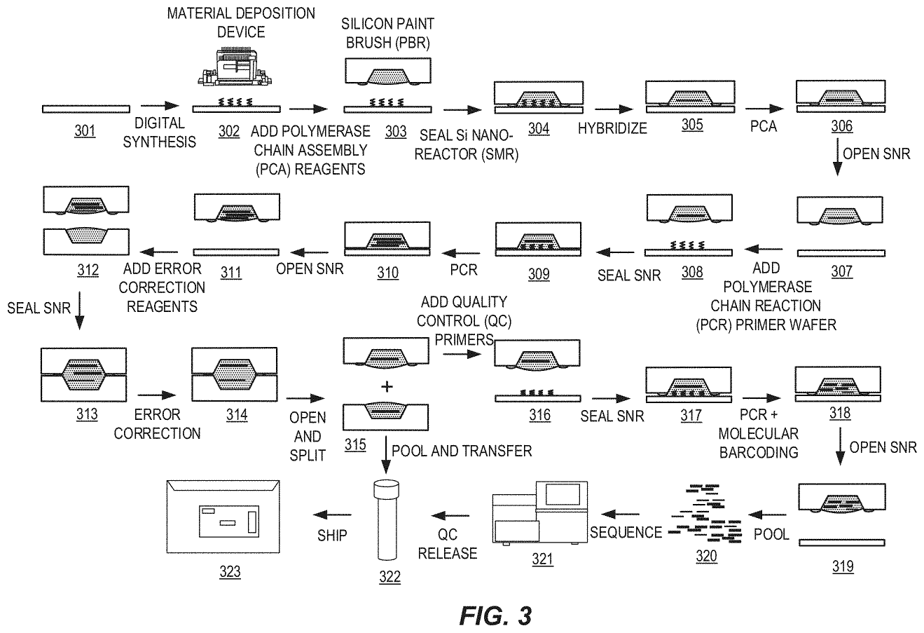

Referring to the Figures, FIG. 3 illustrates an exemplary process workflow for synthesis of nucleic acids (e.g., genes) from shorter nucleic acids. The workflow is divided generally into phases: (1) de novo synthesis of a single stranded nucleic acid library, (2) joining nucleic acids to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large nucleic acids for generation are selected, a predetermined library of nucleic acids is designed for de novo synthesis. Various suitable methods are known for generating high density polynucleotide arrays. In the workflow example, a device surface layer is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 302. In some instances, polynucleotides are cleaved from the surface at this stage. Cleavage includes gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library 303. Prior to or after the sealing 304 of the polynucleotides, a reagent is added to release the polynucleotides from the substrate. In the exemplary workflow, the polynucleotides are released subsequent to sealing of the nanoreactor 305. Once released, fragments of single stranded polynucleotides hybridize in order to span an entire long range sequence of DNA. Partial hybridization 305 is possible because each synthesized polynucleotide is designed to have a small portion overlapping with at least one other polynucleotide in the pool.

After hybridization, a PCA reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for forming a complete large span of double stranded DNA 306.

After PCA is complete, the nanoreactor is separated from the device 307 and positioned for interaction with a device having primers for PCR 308. After sealing, the nanoreactor is subject to PCR 309 and the larger nucleic acids are amplified. After PCR 310, the nanochamber is opened 311, error correction reagents are added 312, the chamber is sealed 313 and an error correction reaction occurs to remove mismatched base pairs and/or strands with poor complementarity from the double stranded PCR amplification products 314. The nanoreactor is opened and separated 315. Error corrected product is next subject to additional processing steps, such as PCR and molecular bar coding, and then packaged 322 for shipment 323.

In some instances, quality control measures are taken. After error correction, quality control steps include for example interaction with a wafer having sequencing primers for amplification of the error corrected product 316, sealing the wafer to a chamber containing error corrected amplification product 317, and performing an additional round of amplification 318. The nanoreactor is opened 319 and the products are pooled 320 and sequenced 321. After an acceptable quality control determination is made, the packaged product 322 is approved for shipment 323.

In some instances, a nucleic acid generated by a workflow such as that in FIG. 3 is subject to mutagenesis using overlapping primers disclosed herein. In some instances, a library of primers are generated by in situ preparation on a solid support and utilize single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 302.

Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

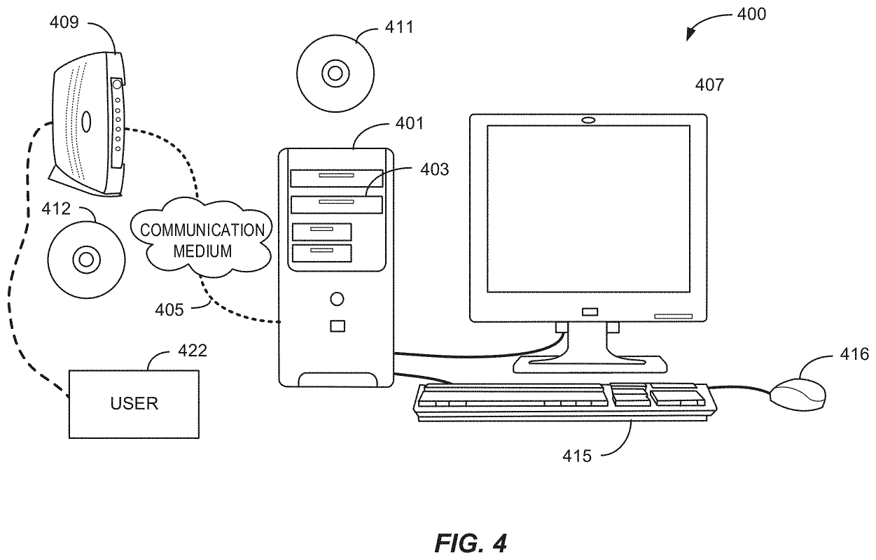

The computer system 400 illustrated in FIG. 4 may be understood as a logical apparatus that can read instructions from media 411 and/or a network port 405, which can optionally be connected to server 409 having fixed media 412. The system, such as shown in FIG. 4 can include a CPU 401, disk drives 403, optional input devices such as keyboard 415 and/or mouse 416 and optional monitor 407. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 422 as illustrated in FIG. 4.

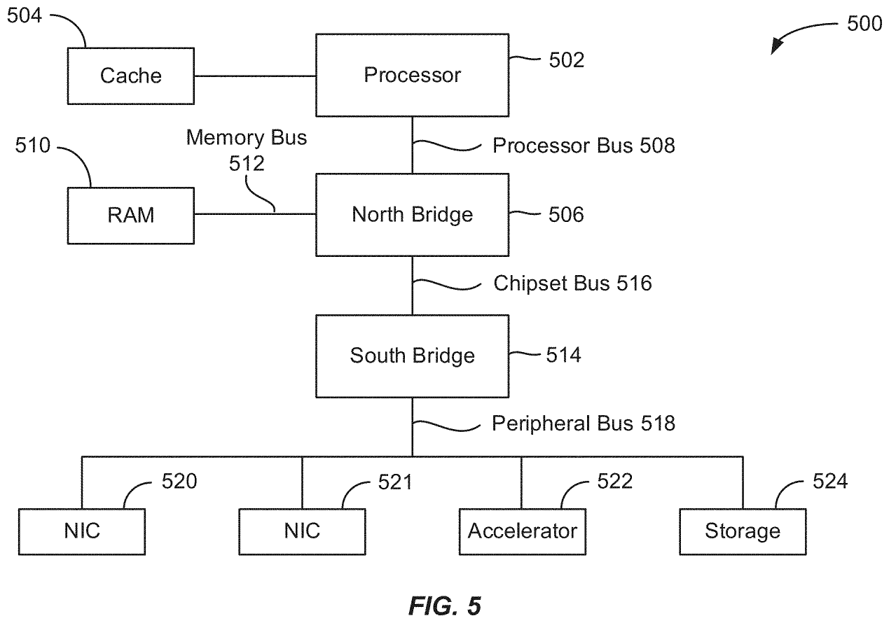

FIG. 5 is a block diagram illustrating a first example architecture of a computer system 500 that can be used in connection with example instances of the present disclosure. As depicted in FIG. 5, the example computer system can include a processor 502 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 5, a high speed cache 504 can be connected to, or incorporated in, the processor 502 to provide a high speed memory for instructions or data that have been recently, or are frequently, used by the processor 502. The processor 502 is connected to a north bridge 506 by a processor bus 508. The north bridge 506 is connected to random access memory (RAM) 510 by a memory bus 512 and manages access to the RAM 510 by the processor 502. The north bridge 506 is also connected to a south bridge 514 by a chipset bus 516. The south bridge 514 is, in turn, connected to a peripheral bus 518. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 518. In some alternative architectures, the functionality of the north bridge can be incorporated into the processor instead of using a separate north bridge chip. In some instances, system 500 can include an accelerator card 522 attached to the peripheral bus 518. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 524 and can be loaded into RAM 510 and/or cache 504 for use by the processor. The system 500 includes an operating system for managing system resources; non-limiting examples of operating systems include: Linux, Windows™, MACOS™, BlackBerry OS™, iOS″, and other functionally-equivalent operating systems, as well as application software running on top of the operating system for managing data storage and optimization in accordance with example instances of the present disclosure. In this example, system 500 also includes network interface cards (NICs) 520 and 521 connected to the peripheral bus for providing network interfaces to external storage, such as Network Attached Storage (NAS) and other computer systems that can be used for distributed parallel processing.

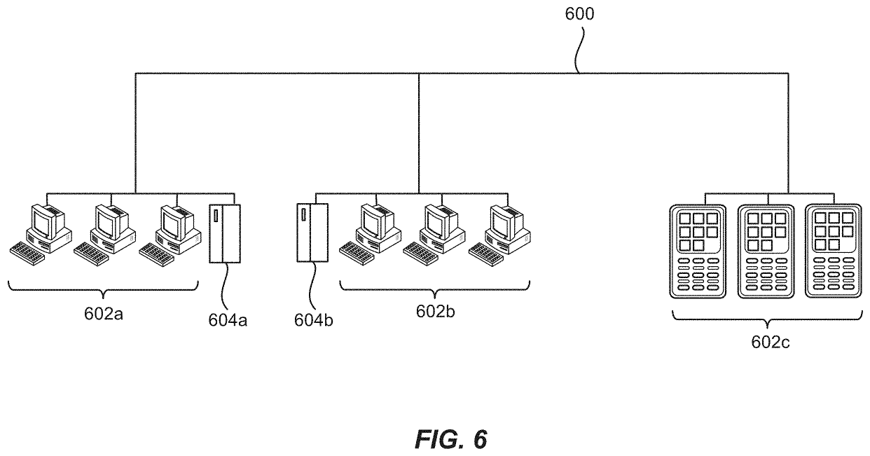

FIG. 6 is a diagram showing a network 600 with a plurality of computer systems 602a, and 602b, a plurality of cell phones and personal data assistants 602c, and Network Attached Storage (NAS) 604a, and 604b. In example instances, systems 602a, 602b, and 602c can manage data storage and optimize data access for data stored in Network Attached Storage (NAS) 604a and 604b. A mathematical model can be used for the data and be evaluated using distributed parallel processing across computer systems 602a, and 602b, and cell phone and personal data assistant systems 602c. Computer systems 602a, and 602b, and cell phone and personal data assistant systems 602c can also provide parallel processing for adaptive data restructuring of the data stored in Network Attached Storage (NAS) 604a and 604b. FIG. 6 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

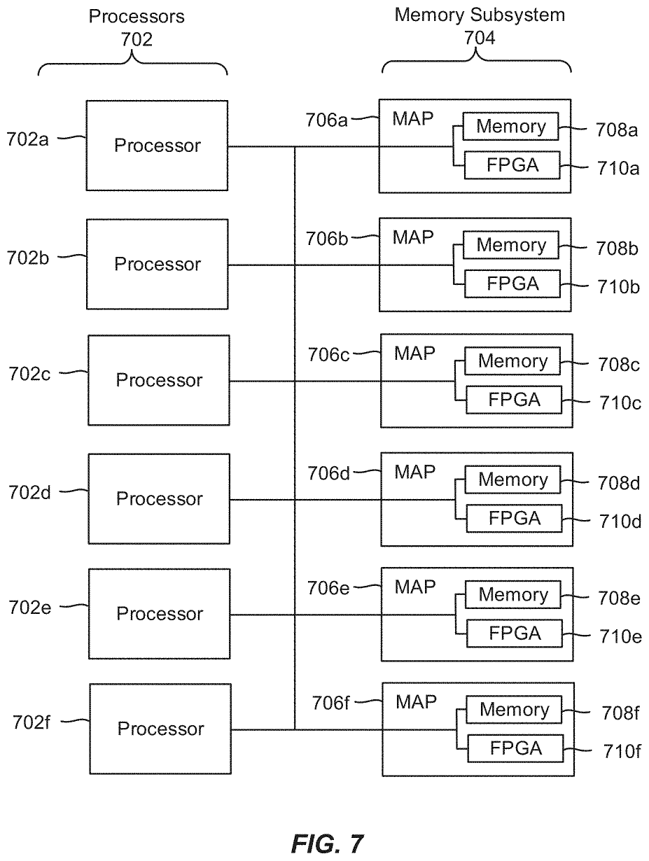

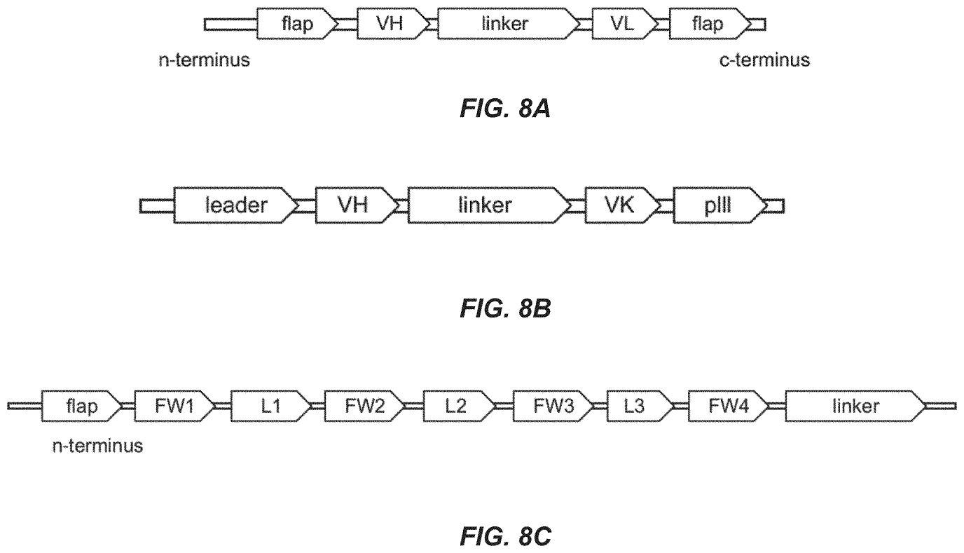

FIG. 7 is a block diagram of a multiprocessor computer system 700 using a shared virtual address memory space in accordance with an example instance. The system includes a plurality of processors 702a-f that can access a shared memory subsystem 704. The system incorporates a plurality of programmable hardware memory algorithm processors (MAPs) 706a-f in the memory subsystem 704. Each MAP 706a-f can comprise a memory 708a-f and one or more field programmable gate arrays (FPGAs) 710a-f The MAP provides a configurable functional unit and particular algorithms or portions of algorithms can be provided to the FPGAs 710a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 708a-f, allowing it to execute tasks independently of, and asynchronously from the respective microprocessor 702a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 5, system on chips (SOCs), application specific integrated circuits (ASICs), or other processing and logic elements. For example, the Set Processor and Optimizer can be implemented with hardware acceleration through the use of a hardware accelerator card, such as accelerator card 522 illustrated in FIG. 5.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Functionalization of a Device Surface

A device was functionalized to support the attachment and synthesis of a library of polynucleotides. The device surface was first wet cleaned using a piranha solution comprising 90% H2504 and 10% H₂O₂ for 20 minutes. The device was rinsed in several beakers with DI water, held under a DI water gooseneck faucet for 5 min, and dried with N2. The device was subsequently soaked in NH₄OH (1:100; 3 mL:300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200× spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A descum process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N2. The functionalized surface was activated to serve as a support for polynucleotide synthesis.

### Example 2: Synthesis of a 50-Mer Sequence on an Oligonucleotide Synthesis Device

A two dimensional oligonucleotide synthesis device was assembled into a flowcell, which was connected to a flowcell (Applied Biosystems (ABI394 DNA Synthesizer”). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 3. 5′AGACAATCAACCATTTGGGGTGGACAGCCTTGACCTCTAGACTTCGGCAT ##TTTTTT TTTT3′ (SEQ ID NO.: 3), where # denotes Thymidine-succinyl hexamide CED phosphoramidite (CLP-2244 from ChemGenes), which is a cleavable linker enabling the release of oligos from the surface during deprotection.

The synthesis was done using standard DNA synthesis chemistry (coupling, capping, oxidation, and deblocking) according to the protocol in Table 3 and an ABI synthesizer.

TABLE 3
Synthesis protocols

| General DNA Synthesis | Table 3 | |
|---|---|---|
| Process Name | Process Step | Time (sec) |
| WASH (Acetonitrile | Acetonitrile System Flush | 4 |
| Wash Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | |
| | Phosphoramidite to | 6 |
| | Flowcell | |
| | Activator to Flowcell | 0.5 |
| | Activator + | |
| | Phosphoramidite to | 5 |
| | Flowcell | |
| | Activator to Flowcell | 0.5 |
| | Activator + | |
| | Phosphoramidite to | 5 |
| | Flowcell | |
| | Activator to Flowcell | 0.5 |
| | Activator + | |
| | Phosphoramidite to | 5 |
| | Flowcell | |
| | Incubate for 25 sec | 25 |
| WASH (Acetonitrile | Acetonitrile System Flush | 4 |
| Wash Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile | Acetonitrile System Flush | 4 |
| Wash Flow) | Acetonitrile to Flowcell | 15 |
| | N2 System Flush | 4 |
| | Acetonitrile System Flush | 4 |
| CAPPING (CapA + | CapA + B to Flowcell | 15 |
| B, 1:1, Flow) | | |
| WASH (Acetonitrile | Acetonitrile System Flush | 4 |
| Wash Flow) | Acetonitrile to Flowcell | 15 |
| | Acetonitrile System Flush | 4 |
| OXIDATION | Oxidizer to Flowcell | 18 |
| (Oxidizer Flow) | | |
| WASH (Acetonitrile | Acetonitrile System Flush | 4 |
| Wash Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING | Deblock to Flowcell | 36 |
| (Deblock Flow) | | |
| WASH (Acetonitrile | Acetonitrile System Flush | 4 |
| Wash Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M 12 in 20% pyridine, 10% water, and 70% THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to ˜50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip.

### Example 3: Synthesis of a 100-Mer Sequence on an Oligonucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCATG CTAGCCATACCATGATGATGATGATGATGAGAACCCCGCAT ##TTTTTTTTTT3′, where # denotes Thymidine-succinyl hexamide CED phosphoramidite (CLP-2244 from ChemGenes); SEQ ID NO.: 4) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument.

All ten samples from the two chips were further PCR amplified using a forward (5′ATGCGGGGTTCTCATCATC3′; SEQ ID NO.: 5) and a reverse (5′CGGGATCCTTATCGTCATCG3; SEQ ID NO.: 6) primer in a 50 uL PCR mix (25 uL NEB Q5 mastermix, 2.5 uL 10 uM Forward primer, 2.5 uL 10 uM Reverse primer, 1 uL polynucleotide extracted from the surface, and water up to 50 uL) using the following thermalcycling program:

98° C., 30 sec

98° C., 10 sec; 63° C., 10 sec; 72° C., 10 sec; repeat 12 cycles

72° C., 2 min

The PCR products were also run on a BioAnalyzer, demonstrating sharp peaks at the 100-mer position. Next, the PCR amplified samples were cloned, and Sanger sequenced. Table 4 summarizes the results from the Sanger sequencing for samples taken from spots 1-5 from chip 1 and for samples taken from spots 6-10 from chip 2.

TABLE 4
Sequencing results

| Spot | Error rate | Cycle efficiency |
|---|---|---|
| 1 | 1/763 bp | 99.87% |
| 2 | 1/824 bp | 99.88% |
| 3 | 1/780 bp | 99.87% |
| 4 | 1/429 bp | 99.77% |
| 5 | 1/1525 bp | 99.93% |
| 6 | 1/1615 bp | 99.94% |
| 7 | 1/531 bp | 99.81% |
| 8 | 1/1769 bp | 99.94% |
| 9 | 1/854 bp | 99.88% |
| 10 | 1/1451 bp | 99.93% |

Thus, the high quality and uniformity of the synthesized polynucleotides were repeated on two chips with different surface chemistries. Overall, 89% of the 100-mers that were sequenced were perfect sequences with no errors, corresponding to 233 out of 262.

Table 5 summarizes error characteristics for the sequences obtained from the polynucleotide samples from spots 1-10.

TABLE 5
Error characteristics

| Sample ID/ | OSA_ | OSA_ | OSA_ | OSA_ | OSA_ | OSA_ | OSA_ | OSA_ | OSA_ | OSA_ |
|---|---|---|---|---|---|---|---|---|---|---|
| Spot no. | 0046/1 | 0047/2 | 0048/3 | 0049/4 | 0050/5 | 0051/6 | 0052/7 | 0053/8 | 0054/9 | 0055/10 |
| Total | 32 | 32 | 32 | 32 | 32 | 32 | 32 | 32 | 32 | 32 |
| Sequences | | | | | | | | | | |
| Sequencing | 25 of 28 | 27 of 27 | 26 of 30 | 21 of 23 | 25 of 26 | 29 of 30 | 27 of 31 | 29 of 31 | 28 of 29 | 25 of 28 |
| Quality | | | | | | | | | | |
| Oligo | 23 of 25 | 25 of 27 | 22 of 26 | 18 of 21 | 24 of 25 | 25 of 29 | 22 of 27 | 28 of 29 | 26 of 28 | 20 of 25 |
| Quality | | | | | | | | | | |
| ROI Match | 2500 | 2698 | 2561 | 2122 | 2499 | 2666 | 2625 | 2899 | 2798 | 2348 |
| Count | | | | | | | | | | |
| ROI | 2 | 2 | 1 | 3 | 1 | 0 | 2 | 1 | 2 | 1 |
| Mutation | | | | | | | | | | |
| ROI Multi | 0 | 0 | 0 | 0 | 0 | 0 | 0 | 0 | 0 | 0 |
| Base | | | | | | | | | | |
| Deletion | | | | | | | | | | |
| ROI Small | 1 | 0 | 0 | 0 | 0 | 0 | 0 | 0 | 0 | 0 |
| Insertion | | | | | | | | | | |
| ROI Single | 0 | 0 | 0 | 0 | 0 | 0 | 0 | 0 | 0 | 0 |
| Base | | | | | | | | | | |
| Deletion | | | | | | | | | | |
| Large | 0 | 0 | 1 | 0 | 0 | 1 | 1 | 0 | 0 | 0 |
| Deletion | | | | | | | | | | |
| Count | | | | | | | | | | |
| Mutation: | 2 | 2 | 1 | 2 | 1 | 0 | 2 | 1 | 2 | 1 |
| G > A | | | | | | | | | | |
| Mutation: | 0 | 0 | 0 | 1 | 0 | 0 | 0 | 0 | 0 | 0 |
| T > C | | | | | | | | | | |
| ROI Error | 3 | 2 | 2 | 3 | 1 | 1 | 3 | 1 | 2 | 1 |
| Count | | | | | | | | | | |
| ROI Error | Err: ~1 | Err: ~1 | Err: ~1 | Err: ~1 | Err: ~1 | Err: ~1 | Err: ~1 | Err: ~1 | Err: ~1 | Err: ~1 |
| Rate | in 834 | in 1350 | in 1282 | in 708 | in 2500 | in 2667 | in 876 | in 2900 | in 1400 | in 2349 |
| ROI Minus | MP Err: | MP Err: | MP Err: | MP Err: | MP Err: | MP Err: | MP Err: | MP Err: | MP Err: | MP Err: |
| Primer | ~1 in 763 | ~1 in 824 | ~1 in 780 | ~1 in 429 | ~1 in 1525 | ~1 in 1615 | ~1 in 531 | ~1 in 1769 | ~1 in 854 | ~1 in 1451 |
| Error Rate | | | | | | | | | | |

### Example 4: Design of GPCR-Focused Antibody Library is Based on GPCR Binding Motifs and GPCR Antibodies

This Example describes the design of chemokine receptor antibody libraries.

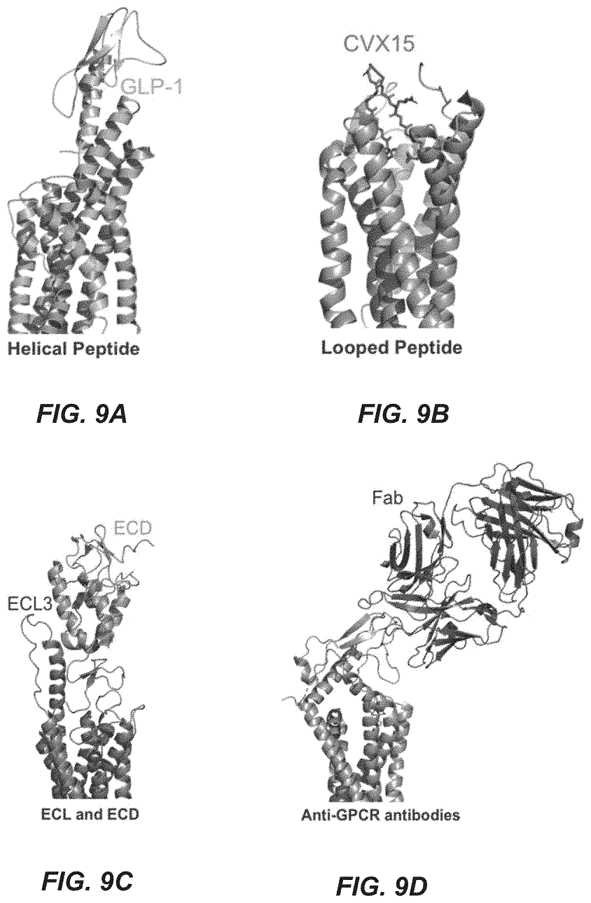

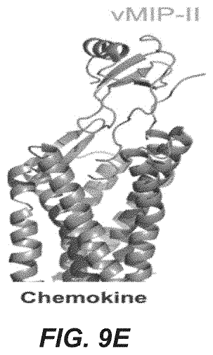

All known GPCR interactions, which include interactions of GPCRs with ligands, peptides, antibodies, endogenous extracellular loops and small molecules were analyzed to map the GPCR binding molecular determinants. Crystal structures of almost 150 peptides, ligand or antibodies bound to ECDs of around 50 GPCRs (http://www.gperdb.org) were used to identify GPCR binding motifs. Over 1000 GPCR binding motifs were extracted from this analysis. In addition, by analysis of all solved structures of GPCRs (zhanglab.ccmb.med.umich.edu/GPCR-EXP/), over 2000 binding motifs from endogenous extracellular loops of GPCRs were identified. Finally, by analysis of structures of over 100 small molecule ligands bound to GPCR, a reduced amino acid library of 5 amino acids (Tyr, Phe, His, Pro and Gly) that may be able to recapitulate many of the structural contacts of these ligands was identified. A sub-library with this reduced amino acid diversity was placed within a CxxxxxC motif. In total, over 5000 GPCR binding motifs were identified (FIGS. 9A-9E). These binding motifs were placed in one of five different stem regions: CARDLRELECEEWTxxxxxSRGPCVDPRGVAGSFDVW, CARDMYYDFxxxxxEVVPADDAFDIW, CARDGRGSLPRPKGGPxxxxxYDSSEDSGGAFDIW, CARANQHFxxxxxGYHYYGMDVW, CAKHMSMQxxxxxRADLVGDAFDVW.

These stem regions were selected from structural antibodies with ultra-long HCDR3s. Antibody germlines were specifically chosen to tolerate these ultra-long HCDR3s. Structure and sequence analysis of human antibodies with longer than 21 amino acids revealed a V-gene bias in antibodies with long CDR3s. Finally, the germline IGHV (IGHV1-69 and IGHV3-30), IGKV (IGKV1-39 and IGKV3-15) and IGLV (IGLV1-51 and IGLV2-14) genes were chosen based on this analysis.

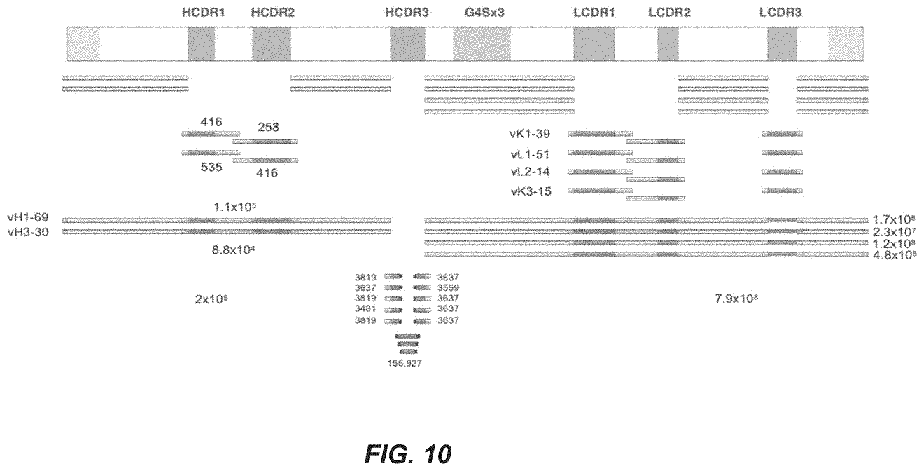

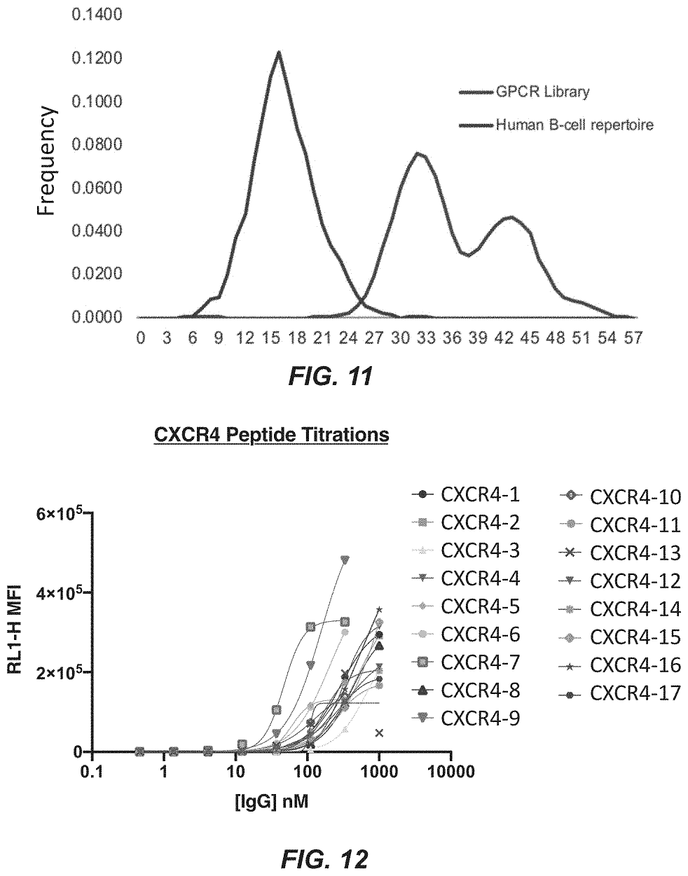

In addition to HCDR3 diversity, limited diversity was also introduced in the other 5 CDRs. There were 416 HCDR1 and 258 HCDR2 variants in the IGHV1-69 domain; 535 HCDR1 and 416 HCDR2 variants in the IGHV3-30 domain; 490 LCDR1, 420 LCDR2 and 824 LCDR3 variants in the IGKV1-39 domain; 490 LCDR1, 265 LCDR2 and 907 LCDR3 variants in the IGKV3-15 domain; 184 LCDR1, 151 LCDR2 and 824 LCDR3 variants in the IGLV1-51 domain; 967 LCDR1, 535 LCDR2 and 922 LCDR3 variants in the IGLV2-14 domain (FIG. 10). These CDR variants were selected by comparing the germline CDRs with the near-germline space of single, double and triple mutations observed in the CDRs within the V-gene repertoire of at least two out of 12 human donors. All CDRs have were pre-screened to remove manufacturability liabilities, cryptic splice sites or nucleotide restriction sites. The CDRs were synthesized as an oligo pool and incorporated into the selected antibody scaffolds. The heavy chain (V_{H}) and light chain (V_{L}) genes were linked by (G₄S)₃ linker. The resulting scFv (V_{H}-linker-V_{L}) gene pool was cloned into a phagemid display vector at the N-terminal of the M13 gene-3 minor coat protein. The final size of the GPCR library is 1×10¹⁰ in a scFv format. Next-generation sequencing (NGS) was performed on the final phage library to analyze the HCDR3 length distribution in the library for comparison with the HCDR3 length distribution in B-cell populations from three healthy adult donors. The HCDR3 sequences from the three healthy donors used were derived from a publicly available database with over 37 million B-cell receptor sequences³¹. The HCDR3 length in the GPCR library is much longer than the HCDR3 length observed in B-cell repertoire sequences. On average, the median HCDR3 length in the GPCR library (which shows a biphasic pattern of distribution) is two or three times longer (33 to 44 amino acids) than the median lengths observed in natural B-cell repertoire sequences (15 to 17 amino acids) (FIG. 11). The biphasic length distribution of HCDR3 in the GPCR library is mainly caused by the two groups of stems (8aa, 9aaxxxxx10aa, 12aa) and (14aa, 16aa xxxxx18aa, 14aa) used to present the motifs within HCDR3.

### Example 5: CXCR4 Variants

This Example shows design and identification of CXCR4 immunoglobulin variants.

CXCR4 variants were designed similarly as described in Example 4. CXCR4-expressing and non-expressing cells were harvested for 0.1-0.2 million cells per sample. Cells were blocked with 1% FBS in PBS for 1 hour at 4 C, and incubated with a 3-fold titration of IgGs or peptides from 100 nM for 1 hour at 4 C. After incubation and washing, cells were incubated with an anti-hIgG secondary-APC labeled at 1:500 dilution for 30 minutes at 4 C, and detected by flow cytometry for cell surface binding. Data is seen in FIG. 12.

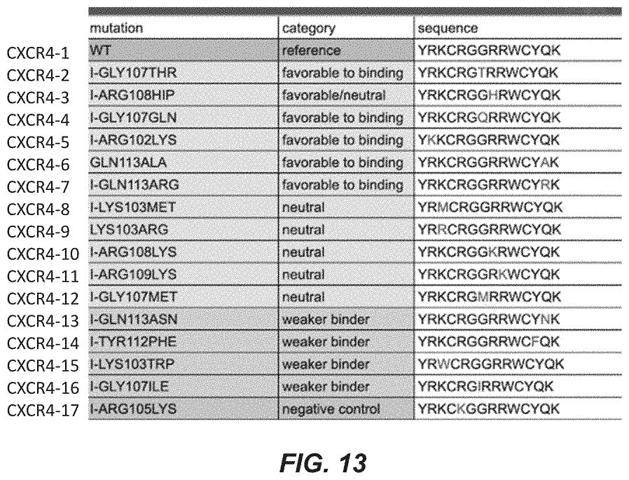

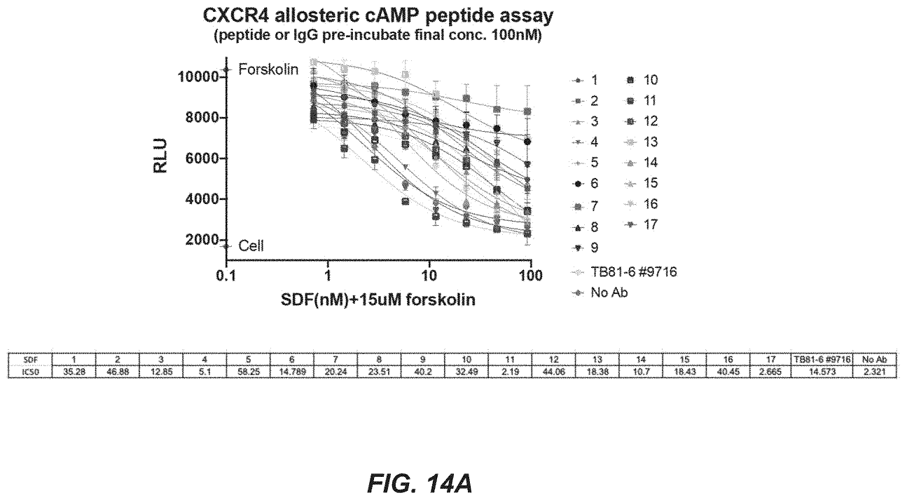

The CXCR4 variants were biotinylated and cyclized using the following format: (Biotin-PEG2)-OH]-GS-YRKCRGGRRWCYQK-NH2. The biotinylated and cyclized sequences are seen in Table 6 and FIG. 13. The CXCR4-249-1 sequence was a result of grafting variant CXCR4-7 (YRKCRGGRRWCYRK) onto CXCR4-81-6 (GSGGYRKCRGGRRWCYRKGGGS) where the CDRH3 of CXCR4-81-6 was replaced with that of CXCR4-7.

TABLE 6

| SEQ ID NO | Variant | Sequence |
|---|---|---|
| 7 | CXCR4-1 | (Biotin-PEG2)-GSYRKCRGGRRWCYQK-amide |
| 8 | CXCR4-2 | (Biotin-PEG2)-GSYRKCRGTRRWCYQK-amide |
| 9 | CXCR4-3 | (Biotin-PEG2)-GSYRKCRGGHRWCYQK-amide |
| 10 | CXCR4-4 | (Biotin-PEG2)-GSYRKCRGQRRWCYQK-amide |
| 11 | CXCR4-5 | (Biotin-PEG2)-GSYKKCRGGRRWCYQK-amide |
| 12 | CXCR4-6 | (Biotin-PEG2)-GSYRKCRGGRRWCYAK-amide |
| 13 | CXCR4-7 | (Biotin-PEG2)-GSYRKCRGGRRWCYRK-amide |
| 14 | CXCR4-8 | (Biotin-PEG2)-GSYRMCRGGRRWCYQK-amide |
| 15 | CXCR4-9 | (Biotin-PEG2)-GSYRRCRGGRRWCYQK-amide |
| 16 | CXCR4-10 | (Biotin-PEG2)-GSYRKCRGGKRWCYQK-amide |
| 17 | CXCR4-11 | (Biotin-PEG2)-GSYRKCRGGRKWCYQK-amide |
| 18 | CXCR4-12 | (Biotin-PEG2)-GSYRKCRGMRRWCYQK-amide |
| 19 | CXCR4-13 | (Biotin-PEG2)-GSYRKCRGGRRWCYNK-amide |
| 20 | CXCR4-14 | (Biotin-PEG2)-GSYRKCRGGRRWCFQK-amide |
| 21 | CXCR4-15 | (Biotin-PEG2)-GSYRWCRGGRRWCYQK-amide |
| 22 | CXCR4-16 | (Biotin-PEG2)-GSYRKCRGIRRWCYQK-amide |
| 23 | CXCR4-17 | (Biotin-PEG2)-GSYRKCKGGRRWCYQK-amide |

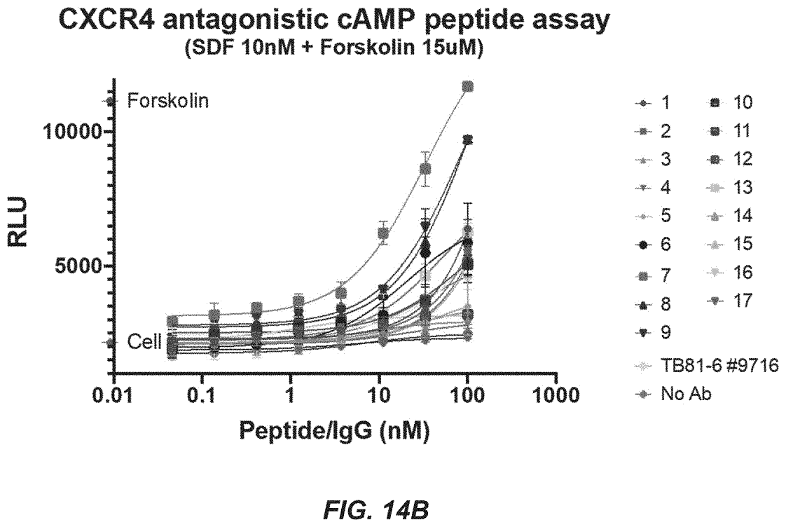

cAMP assays using the CXCR4 variants were performed. The cAMP assays were performed using the cAMP Hunter™ eXpress GPCR Assays according to manufacturer's protocol. Gi-coupled CXCR4 expressing cells were seeded at 15000 cells per well in 96-well plate one day before the assay treatment. Sixteen hours later, the cells were incubated with fixed or titration of IgG from 100 nM at 37 C for 1 hour, followed by forskolin (15 uM) and SDF incubation at 37 C for 30 minutes. cAMP detection reagents were added and the level was detected 16 hours later to evaluate IgG function using DisvocerX PathHunter cAMP detection kit. Data from the cAMP assays are seen in FIGS. 14A-14B.

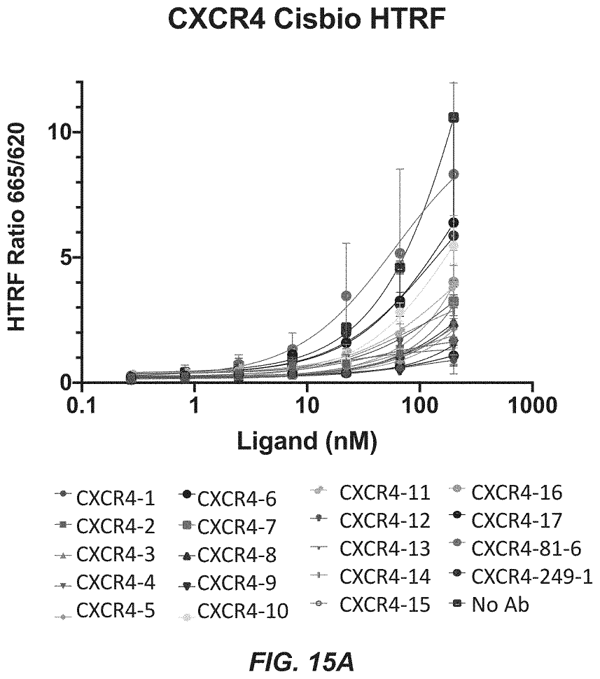

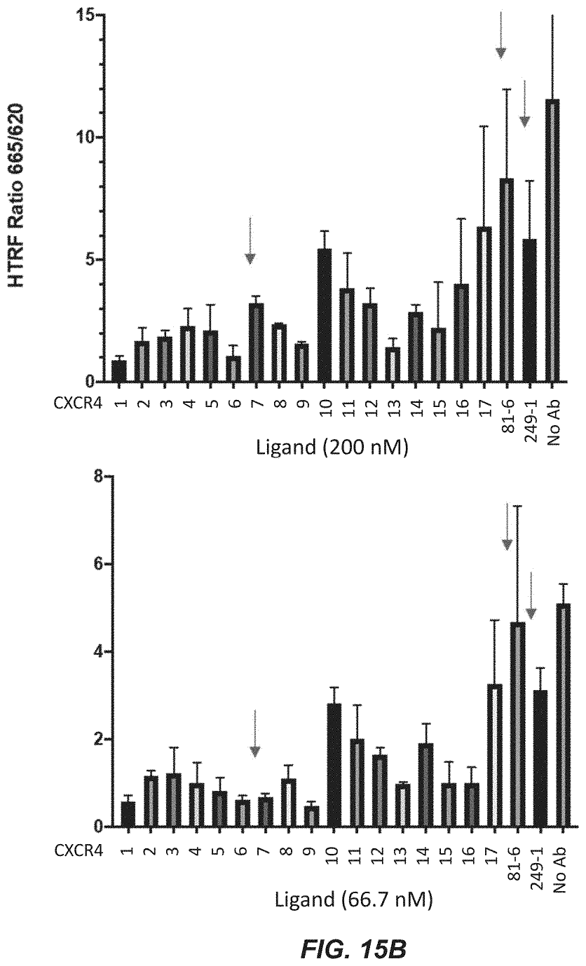

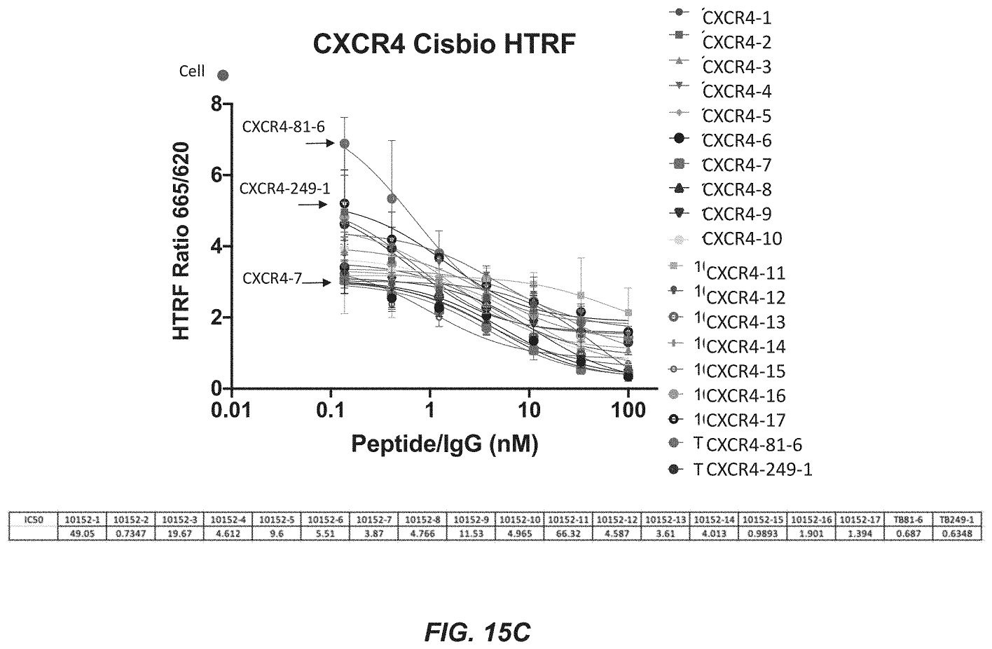

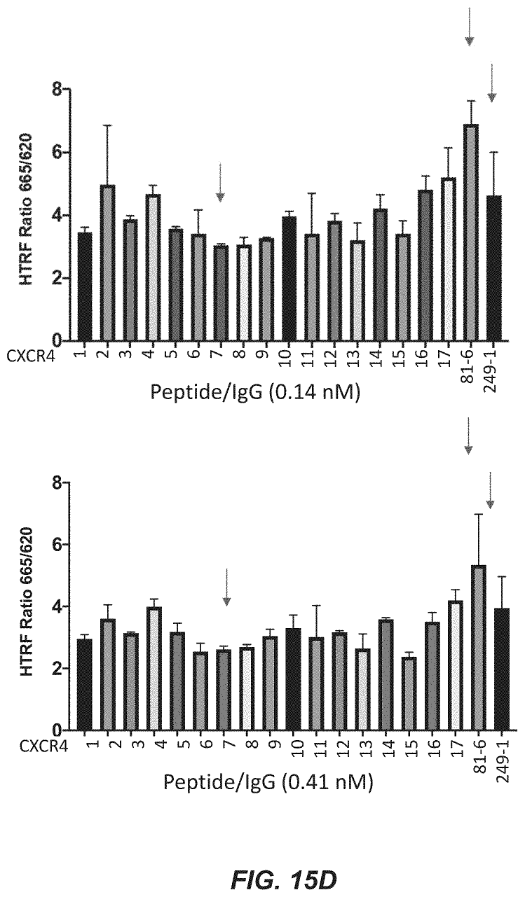

Ligand binding assays with the CXCR4 variants were performed. Briefly, the ligand binding assays were performed using the Tag-lite® Chemokine CXCR4 Receptor Ligand Binding Assay according to the manufacturer's protocol. The Tag-lite® Chemokine CXCR4 cells transiently expressing the chemokine CXCR4 receptor were labeled with Terbium for conducting receptor binding studies on the CXCR4 receptor. Cells were pre-incubated with 100 nM peptides/IgG, followed by radio ligand treatment from 200 nM, 3× titration (FIG. 15A). Various ligand titrations were assayed in the ligand binding assay (FIG. 15B). Cells were pre-incubated with 100 nM peptides/IgG, followed by radio ligand treatment of 50 nM (FIG. 15C). Various peptide/IgG titrations were assayed in the ligand binding assay (FIG. 15D).

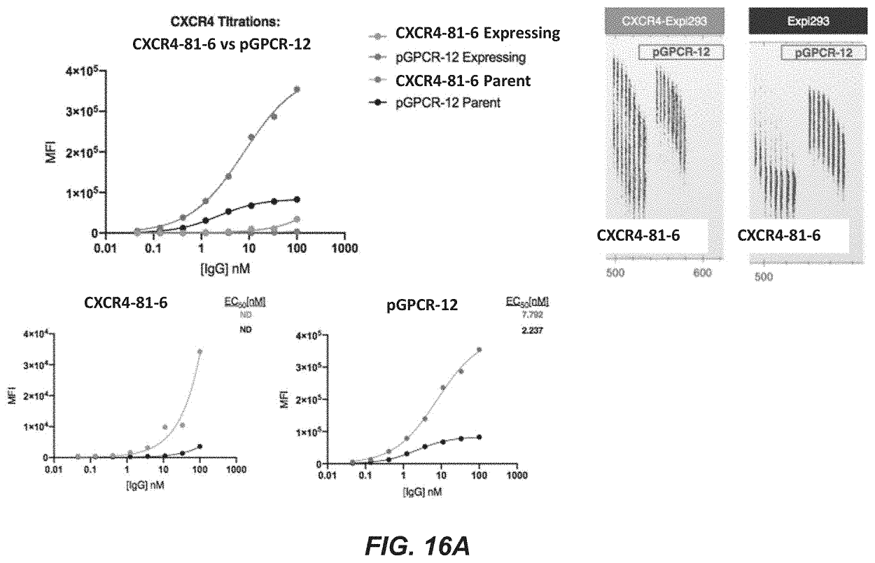

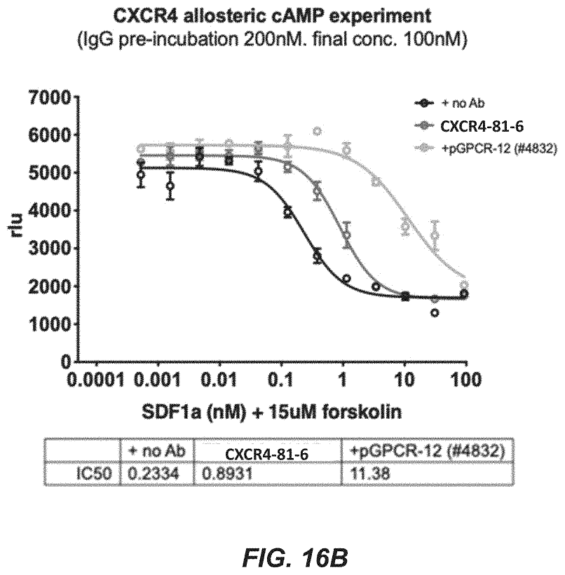

The CXCR4-81-6 variant was tested in flow titration and cAMP assays. Briefly, for the flow titration assay, target expressing and non-expressing cells were incubated with a titration of IgG including CXCR4-81-6 and then detected with an anti-hIgG secondary-APC labeled antibody. pGPCR-12 was used as a control IgG. Data is seen in FIG. 16A. For the cAMP assay, Gi-coupled CXCR4 expressing cells were incubated with IgG, followed by forskolin and SDF treatment. cAMP levels were detected to evaluate IgG function and the IC50 of CXCR4-81-6 was determined to be 0.9 nM (FIG. 16B).

### Example 6. CXCR5 Variants

This Example shows design and identification of CXCR5 immunoglobulin variants.

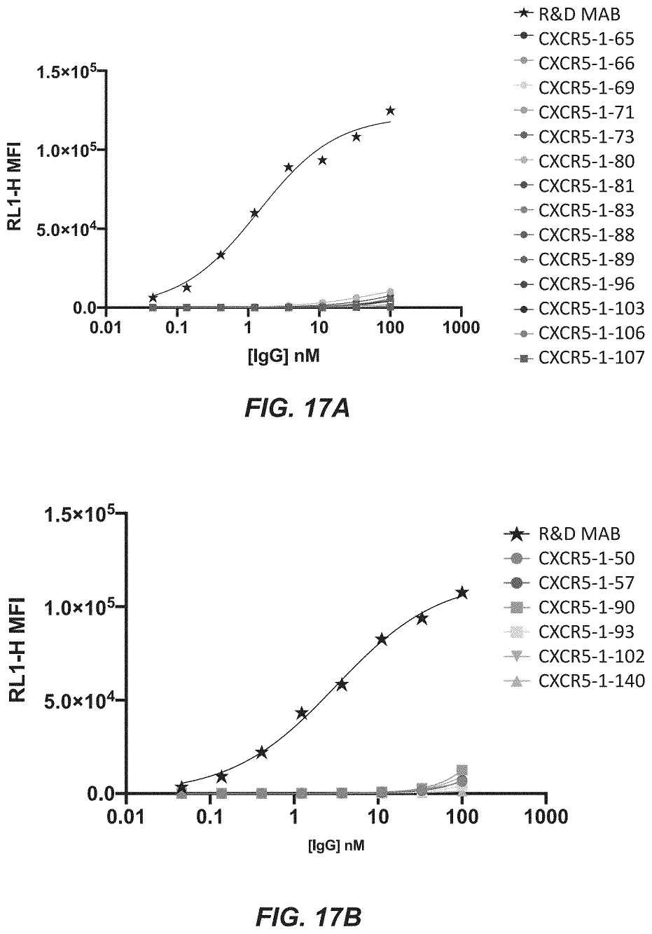

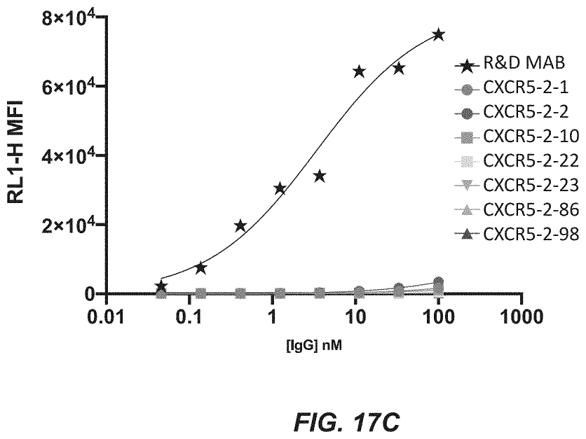

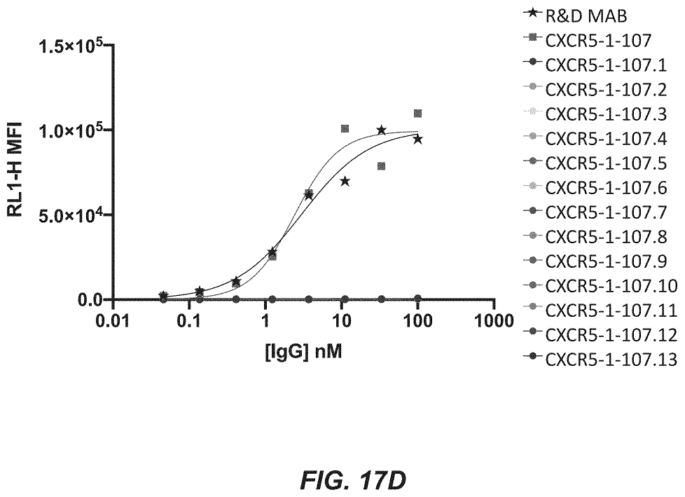

CXCR5 variants were designed similarly as described in Example 4. CXCR5-expressing and non-expressing cells were harvested for 0.1-0.2 million cells per sample. Cells were blocked with 1% FBS in PBS for 1 hour at 4 C, and incubated with a 3-fold titration of IgGs from 100 nM for 1 hour at 4 C. After incubation and washing, cells were incubated with an anti-hIgG secondary-APC labeled at 1:500 dilution for 30 minutes at 4 C, and detected by flow cytometry for cell surface binding. Data is seen in FIGS. 17A-17C.

CXCR5 variant CXCR5-1-107 was used to generate variants and tested in titration assays. The heavy chain from variant CXCR5-1-107 was used. Data is seen in FIG. 17D.

### Example 7. Exemplary Sequences

TABLE 7
CXCR4 Variable Heavy (VH) Chain Sequences

| SEQ | | |
|---|---|---|
| ID NO | Variant | Sequence |
| 24 | CXCR4-81-6 | EVQLVESGGGLVQAGGSLRLSCAASGRTFSSYAMGW |
| | | FRQAPGKEREFVAAISWSGGSTYYADSVKGRFTISAD |
| | | NAKNTVYLQMNSLKPEDTAVYYCAAARGYWRWRL |
| | | GRRYDYWGQGTQVTVSS |
| 25 | CXCR4-249-1 | EVQLVESGGGLVQAGGSLRLSCAASGRTFSSYAMGW |
| | | FRQAPGKEREFVAAISWSGGSTYYADSVKGRFTISAD |
| | | NAKNTVYLQMNSLKPEDTAVYYCGSGGYRKCRGGR |
| | | RWCYRKGGGSWGQGTQVTVSS |
| 26 | CXCR4-12 | EVQLVESGGGLVQPGGSLRLSCAASGFTFSDYYMSW |
| | | VRQAPGKGLEWVGFIRHKANFETTEYSTSVKGRFTIS |
| | | RDDSKNSLYLQMNSLKTEDTAVYYCARDLPGFAYW |
| | | GQGTLVTVSS |
| 27 | CXCR4-81-5 | EVQLVESGGGLVQAGGSLRLSCAASGRTFSSYGMGW |
| | | FRQAPGKERELVAAINWSGGRTSYADSVKGRFTISAD |
| | | NAKNTVYLQMNSLKPEDTAVYYCATGRGYWRWRLG |
| | | RAYDYWGQGTQVTVSS |
| 28 | CXCR4-81-9 | EVQLLESGGGLVQPGGSLRLSCAASGFTFRSYATSWV |
| | | RQAPGKGLEWVSTISGSGGSTHYADSVKGRFTISRDN |
| | | SKNTLYLQMNSLRAEDTAVYYCARGPRRWLLSRARG |
| | | SFDIWGQGTLVTVSS |

TABLE 8
CXCR4 Variable Light (VL) Chain Sequences

| SEQ | | |
|---|---|---|
| ID NO | Variant | Sequence |
| 29 | CXCR4-81-6 | DIQMTQSPSSLSASVGDRVTITCRASQSVTTYLNWYQ |
| | | QKPGKAPKLLIYGSSNLQSGVPSRFSGSGSGTDFTLTIS |
| | | SLQPEDFATYYCQQGYSTPWTFGGGTKVEIKR |
| 30 | CXCR4-249-1 | DIQMTQSPSSLSASVGDRVTITCRASQSVTTYLNWYQ |
| | | QKPGKAPKLLIYGSSNLQSGVPSRFSGSGSGTDFTLTIS |
| | | SLQPEDFATYYCQQGYSTPWTFGGGTKVEIKR |
| 31 | CXCR4-12 | DIVMTQSPDSLAVSLGERATINCKSSQSLFNSHTRKNY |
| | | LAWYQQKPGQPPKLLIYWASARGSGVPDRFSGSGSGT |
| | | DFTLTISSLQAEDVAVYYCKQSFNLRTFGGGTKVEIK |
| 32 | CXCR4-81-5 | DIQMTQSPSSLSASVGDRVTITCRASQNIASYLNWYQ |
| | | QKPGKAPKLLIYAASTLQGGVPSRFSGSGSGTDFTLTI |
| | | SSLQPEDFATYYCQQSYSLPYTFGGGTKVEIK |
| 33 | CXCR4-81-9 | DIQMTQSPSSLSASVGDRVTITCRASQSIGGYLNWYQ |
| | | QKPGKAPKLLIYAASRLQSGVPSRFSGSGSGTDFTLTIS |
| | | SLQPEDFATYYCQQSHSFPRTFGGGTKVEIK |

TABLE 9
CXCR5 Variable Heavy (VH) Chain Sequences

| SEQ | | |
|---|---|---|
| ID NO | Variant | Sequence |
| 34 | CXCR5-1-1 | EVQLVESGGGLVQPGGSLRLSCAASGFTFSDYWMHWVRQAPGK |
| | | GLEWVSVISPDGSITYYPDSVKGRFTISRDNSKNTLYLQMNSLRA |
| | | EDTAVYYCARKDVWVIFSTHDGAYGFDVWGQGTLVTVSS |
| 35 | CXCR5-1-2 | EVQLVESGGGLVQPGGSLRLSCAASGRAFIAYAMGWFRQAPGK |
| | | EREMVAAISWSGGITWYADSVKGRFTISADNSKNTAYLQMNSL |
| | | KPEDTAVYYCAAASPGGAINYGRGYDWGQGTLVTVSS |
| 36 | CXCR5-1-3 | EVQLVESGGGLVQPGGSLRLSCAASGFTFSSYGMHWVRQAPGK |
| | | GLEWVAVISPNGGNTYYPDSVKGRFTISRDNSKNTLYLQMNSLR |
| | | AEDTAVYYCARHDHDYYAFDYWGQGTLVTVSS |
| 37 | CXCR5-1-4 | EVQLVQSGAEVKKPGSSVKVSCKASGGTFSSYAMSWVRQAPGQ |
| | | GLEWIGTINPGDGYTHYADKFKGRVTITRDTSTSTVYMELSRLRS |
| | | EDTAVYYCARHTSSNGVYSTWFAYWGQGTLVTVSS |
| 38 | CXCR5-1-5 | EVQLVESGGGLVQPGGSLRLSCAASGGTFSLYAMGWFRQAPGK |
| | | EREFVAAISWSGGSTIYADSVKGRFTISADNIKNTAYLHMNSLKP |
| | | EDTAVYYCASNESDAYNWGQGTLVTVSS |
| 39 | CXCR5-1-6 | EVQLVESGGGLVQPGGSLRLSCAASGFTFSSYGMHWVRQAPGK |
| | | GLEWVAYISYSGGEKYYADSVKGRFTISRDNSKNTLYLQMNSLR |
| | | AEDTAVYYCARDDDGGDAFDYWGQGTLVTVSS |
| 40 | CXCR5-1-7 | EVQLVESGGGLVQPGGSLRLSCAASGRAFIAYAMGWFRQAPGK |
| | | EREMVAAISWSGGITWYADSVKGRFTISADNSKNTAYLQMNSL |
| | | KPEDTAVYYCAAASPGGAINYGRGYDWGQGTLVTVSS |
| 41 | CXCR5-1-8 | EVQLVQSGAEVKKPGSSVKDSCKASGGTFSDYAMSWVRQAPGQ |
| | | GLEWIGRINPYDGYTHYNDKFKGRGTITRDTSTSTVYMELSSLRS |
| | | EDTAVYYCARDYSSSFVFHAMDYWGQGTLVTVSS |
| 42 | CXCR5-1-9 | EVQLVESGGGLVQPGGSLRLSCAASGFTFSDYAMGWVRQAPGK |
| | | GLEWVSYISYDGSNTYYPDSVKGRFTISRDNSKNTLYLQMNSLR |
| | | AEDTAVYYCARIRTNYFGFDYWGQGTLVTVSS |
| 43 | CXCR5-1-10 | EVQLVESGGGLVQPGGSLRLSCAASGRAFIAYAMGWFRQAPGK |
| | | EREMVAAISWSGGITWYADSVKGRFTISADNSKNTAYLQMNSL |
| | | KPEDTAVYYCAAASPGGAINYGRGYDWGQGTLVTVSS |
| 44 | CXCR5-1-11 | EVQLVESGGGLVQPGGSLRLSCAASGRAFIAYAMGWFRQAPGK |
| | | EREMVAAISWSGGITWYADSVKGRFTISADNSKNTAYLQMNSL |
| | | KPEDTAVYYCAAASPGGAINYGRGYDWGQGTLVTVSS |
| 45 | CXCR5-1-12 | EVQLVESGGGLVQPGGSLRLSCAASGFTFSDYAMSLVRQAPGKG |
| | | LEWVSVISYSGSETYYPDSVKGRFTISRDNSKNTLYLQMNSLRAE |
| | | DTAVYYCARHLTNYDPFDYWGQGTLVTVSS |
| 46 | CXCR5-1-13 | EVQLVESGGGLVQPGGSLRLSCAASGFTFSDYGMSWVRQAPGK |
| | | GLEWVSYISPDSVKGRFTISRDNSKNTLYLQMNSLRAEDTAVYY |
| | | CARGDTNWFAFDYWGQGTLVTVSS |
| 47 | CXCR5-1-14 | EVQLVESGGGLVQPGGSLRLSCAASGFTFSNYAMHWVRQAPGK |
| | | GLEWVSVISPNGGNKYYPDSVKGRFTISRDNSKNTLYLQMNSLR |
| | | AEDTAWYYCARILTGGYPFDYWGQGTLVTVSS |
| 48 | CXCR5-1-15 | EVQLVESGGGLVQPGGSLRLSCAASGGTFSLYAMGWFRQAPGK |
| | | EREFVAAISWSGGSTIYADSVKGRFTISADNSKNTAYLQMNSLKP |
| | | EDTAVYYCASNESDAYNWGQGTLVTVSS |
| 49 | CXCR5-1-16 | EVQLVESGGGLVQPGGSLRLSCAASGFTFSDYGMGWVRQAPGK |
| | | GLEWVSVISYDGSNTYYPDSVKGRFTISRDNSKNTLYLQMNSLR |
| | | AEDTAVYYCARHRHYGYPFDYWGQGTLVTVSS |
| 50 | CXCR5-1-17 | EVKLVESGGGLVQPGGSLRLSCAASGFTFSNYAMSWVRQAPGK |
| | | GLEWVAVISYSGGITYYADSVKGRFTISRDNSKNTLYLQMNSLR |
| | | AEDTAVYYCARHRHYNYAFDYWGQGTLVTVSS |
| 51 | CXCR5-1-18 | EVQLVQSGAEVKKPGSSVKVSCKASGGTFSDYAMHWVRQAPG |
| | | QGLEWIGRIRPGDGYTHYADKFKGRVTITRDTSTSTVYMELSSLR |
| | | SEDTAVYYCARFGHSGRSFAYWGQGTLVTVSS |
| 52 | CXCR5-1-19 | EVQLVESGGGLVQPGGSLRLSCAASGFTFSSYYMHWVRQAPGK |
| | | GLEWVAVISPSGGNKYYPDSVKGRFTISRDNSKNTLYLQMNSLR |
| | | AEDTAVYYCARGKDDRLDYLGYYFDYWGQGTLVTVSS |
| 53 | CXCR5-1-20 | EVQLVESGGGLVQPGGSLRLSCAASGFTFSDYWMGWVRQAPGK |
| | | GLEWVSVISPDGGNKYYADSVKGRFTISRDNSKNTLYLQMNSLR |
| | | AEDTAVYYCARHLDGGDGFDYWGQGTLVTVSS |
| 54 | CXCR5-1-21 | EVQLVESGGGLVQPGGSLRLSCAASGFTFSDYWMSWVRQAPGK |
| | | GLEWVAVISYDGSETYYPDSVKGRFTISRDNSKNTLYLQMNSLR |
| | | AEDTAVYYCARDDRGYFGFDYWGQGTLVTVSS |
| 55 | CXCR5-1-22 | EVQLVESGGGLVQPGGSLRLSCAASGFTFSDYWMGWVRQAPGK |
| | | GLEWVAYISYSGSIKYYADSVKGRFTISRDNSKNTLYLQMNSLR |
| | | AEDTAVYYCARPSYLDSVYGHDGYYTLDVWGQGTLVTVSS |
| 56 | CXCR5-1-23 | EVQLVQSGAEVKKPGSSVKVSCKASGGTFSSYAMSLVRQAPGQ |
| | | GLEWIGTIRPGDGYTHYADKFKGRVTITRDTSTSTVYMELSSLRS |
| | | EDTAVYYCARSLLPNTVTAYMDYWGQGTLVTVSS |
| 57 | CXCR5-1-24 | EVQLVESGGGLVQPGGSLRLSCAASGFTFSSYWMGWVRQAPGK |
| | | GLEWVAYISYDGGITYYPDSVKGRFTISRDNSKNTLYLQMNSLR |
| | | AEDTAVYYCARDDDGWYPFDYWGQGTLVTVSS |
| 58 | CXCR5-1-25 | EVQLVQSGAEVKKPGSSVKVSCKASGGTFSSYWMSWVRQAPG |
| | | QGLEWIGVIRPYDGYTYYAQKFKGRVTITRDTSTSTVYMELSSL |
| | | RSEDTAVYYCARHGYKSNYLSYMDYWGQGTLVTVSS |
| 59 | CXCR5-1-26 | EVQLVESGGGLVQPGGSLRLSCAASGFTFSSYAMGWVRQAPGK |
| | | GLEWVSVISYSGGNTYYPDSVKGRFTISRDNSKNTLYLQMNSLR |
| | | AEDTAVYYCARDDHGWYPFDYWGQGTLVTVSS |
| 60 | CXCR5-1-27 | EVQLVESGGGLVQPGGSLRLSCAASGFTFSDYAMHWVRQAPGK |
| | | GLEWVSYISPSGSIKYYADSVKGRFTISRDNSKNTLYLQMNSLRA |
| | | EDTAVYYCARGRHNNFGFDYWGQGTLVTVSS |
| 61 | CXCR5-1-28 | EVQLVESGGGLVQPGGSLRLSCAASGFTFSNYYMGWVRQAPGK |
| | | GLEWVAYISYDGSIKYYPDSVKGRFTISRDNSKNTLYLQMNSLR |
| | | AEDTAVYYCARILDYYFPFDYWGQGTLVTVSS |
| 62 | CXCR5-1-29 | EVQLVQSGAEVKKPGSSVKVSCKASGGTFSNYYMNWVRQAPG |
| | | QGLEWIGRIRPGNGYTHYADKFKGRVTITRDTSTSTVYMELSSLR |
| | | SEDTAVYYCARSESSFYVYQTAFAYWGQGTLVTVSS |
| 63 | CXCR5-1-30 | EVQLVQSGAEVKKPGSSVKVSCKASGGTFSSYAMSWVRQAPGQ |
| | | GLEWIGVIRPGDGYTKYADKFKGRVTITRDTSTSTVYMELSSLRS |
| | | EDTAVYYCARSGLWYNVFNAMDYWGQGTLVTVSS |
| 64 | CXCR5-1-31 | EVQLVESGGGLVQPGGSLRLSCAASGFTFSDYYMGWVRQAPGK |
| | | GLEWVAYISPSGGNKYYPDSVKGRFTISRDNSKNTLYLQMNSLR |
| | | AEDTAVYYCARKSHYFGFWGNNGARTFDYWGQGTLVTVSS |
| 65 | CXCR5-1-32 | EVQLVESGGGLVQPGGSLRLSCAASGFTFSDYGMSWVRQAPGK |
| | | GLEWVAVISYDGSNKYYADSVKGRFTISRDNSKNTLYLQMNSL |
| | | RAEDTAVYYCARGELNRGDRYGYRYHKHRGMDVWGQGTLVT |
| | | VSS |
| 66 | CXCR5-1-33 | EVQLVQSGAEVKKPGSSVKVSCKASGGTFSNYYMNWVRQAPG |
| | | QGLEWIGTIRPNNGETKYNDKFKGRVTITRDTSTSTVYMELSSLR |
| | | SEDTAVYYCARDLYWNFGGYAMDYWGQGTLVTVSS |
| 67 | CXCR5-1-34 | EVQLVQSGAEVKKPGSSVKVSCKASGGTFSDYAMHWVRQAPG |
| | | QGLEWIGVINPNDGYTKYAQKFKGRVTITRDTSTSTVYMELSSL |
| | | RSEDTAVYYCARSFFYYHYGAFDYWGQGTLVTVSS |
| 68 | CXCR5-1-35 | EVQLVESGGGLVQPGGSLRLSCAASGFTFSSYWMHWVRQAPGK |
| | | GLEWVAVISYDGGNKYYPDSVKGRFTISRDNSKNTLYLQMNSL |
| | | RAEDTAVYYCARIDGYYIRWTYYHARTFDYWGQGTLVTVSS |
| 69 | CXCR5-1-36 | EVQLVQSGAEVKKPGSSVKVSCKASGGTFSSYAMNWVRQAPGQ |
| | | GLEWIGTIRPNNGETKYNQKFKGRVTITRDTSTSTVYMELSSLRS |
| | | EDTAVYYCARPSRPSHYSAFSHPYYMDYWGQGTLVTVSS |
| 70 | CXCR5-1-37 | EVQLVESGGGLVQPGGSLRLSCAASGFTFSDYAMHWVRQAPGK |
| | | GLEWVAYISYSGSNKYYPDSVKGRFTISRDNSKNTLYLQMNSLR |
| | | AEDTAVYYCARGPQSWYGLWGQNFDYWGQGTLVTVSS |
| 71 | CXCR5-1-38 | EVQLVESGGGLVQPGGSLRLSCAASGFTFSNYYMSWVRQAPGK |
| | | GLEWVSVISPSGSETYYADSVKGRFTISRDNSKNTLYLQMNSLR |
| | | AEDTAVYYCARHLDNGFPFDYWGQGTLVTVSS |
| 72 | CXCR5-1-39 | EVQLVESGGGLVQPGGSLRLSCAASGFTFSDYAMHWVRQAPGK |
| | | GLEWVSVISYDGSITYYPDSVKGRFTISRDNSKNTLYLQMNSLRA |
| | | EDTAVYYCARIRHRFILWRNYGARGMDYWGQGTLVTVSS |
| 73 | CXCR5-1-40 | EVQLVESGGGLVQPGGSLRLSCAASGFTFSSYWMSWVRQAPGK |
| | | GLEWVSVISPSGSETYYADSVKGRFTISRDNSKNTLYLQMNSLR |
| | | AEDTAVYYCARDRDRYLDLHRYPFDYWGQGTLVTVSS |
| 74 | CXCR5-1-41 | EVQLVQSGAEVKKPGSSVKVSCKASGGTFSSYAMNWVRQAPGQ |
| | | GLEWIGRINPNNGYTHYADKFKGRVTITRDTSTSTVYMELSSLRS |
| | | EDTAVYYCARLLSKSNNLHAMDYWGQGTLVTVSS |
| 75 | CXCR5-1-42 | EVQLVQSGAEVKKPGSSVKVSCKASGGTFSSYWMSWVRQAPG |
| | | QGLEWIGVIRPGNGYTYYNQKFKGRVTITRDTSTSTVYMELSSL |
| | | RSEDTAVYYCARGGAYYYTSITSHGFQFDYWGQGTLVTVSS |
| 76 | CXCR5-1-43 | EVQLVQSGAEVKKPGSSVKVSCKASGGTFSDYWMHWVRQAPG |
| | | QGLEWIGRIRPYDGYTKYNQKFKGRVTITRDTSTSTVYMELSSLR |
| | | SEDTAVYYCARSTIGYDYGYYGFDYWGQGTLVTVSS |
| 77 | CXCR5-1-44 | EVQLVESGGGLVQPGGSLRLSCAASGFTFSDYAMGWVRQAPGK |
| | | GLEWVSANKYYADSVKGRFTISRDNSKNTLYLQMNSLRAEDTA |
| | | VYYCARRVYWDGFYTQDYYYTLDVWGQGTLVTVSS |
| 78 | CXCR5-1-45 | EVQLVESGGGLVQPGGSLRLSCAASGFTFSDYWMHWVRQAPGK |
| | | GLEWVAYISYNGGITYYPDSVKGRFTISRDNSKNTLYLQMNSLR |
| | | AEDTAVYYCARDTSSWTPLLTFYFDYWGQGTLVTVSS |
| 79 | CXCR5-1-46 | EVQLVESGGGLVQPGGSLRLSCAASGFTFSDYAMHWVRQAPGK |
| | | GLEWVSYISYDGSETYYADSVKGRFTISRDNSKNTLYLQMNSLR |
| | | AEDTAVYYCARHLHDNDAFDYWGQGTLVTVSS |
| 80 | CXCR5-1-47 | EVQLVESGGGLVQPGGSLRLSCAASGFTFSNYGMHWVRQAPGK |
| | | GLEWVAYISYSGGITYYPDSVKGRFTISRDNSKNTLYLQMNSLR |
| | | AEDTAVYYCARHRTDGYPFDYWGQGTLVTVSS |
| 81 | CXCR5-1-48 | EVQLVESGGGLVQPGGSLRLSCAASGFTFSNYWMHWVRQAPGK |
| | | GLEWVSVISYSGGNKYYPDSVKGRFTISRDNSKNTLYLQMNSLR |
| | | AEDTAVYYCARKDGLYDRSGYRHARTFDYWGQGTLVTVSS |
| 82 | CXCR5-1-49 | EVQLVESGGGLVQPGGSLRLSCAASGFTFSDYWMHWVRQAPGK |
| | | GLEWVSYISPSGGEKYYADSVKGRFTISRDNSKNTLYLQMNSLR |
| | | AEDTAVYYCARDEDYYYDGSRFNGGYYGPMDVWGQGTLVTVS |
| | | S |
| 83 | CXCR5-1-50 | EVQLVESGGGLVQPGGSLRLSCAASGFTFSDYWMSWVRQAPGK |
| | | GLEWVAYISPSGGNKYYPDSVKGRFTISRDNSKNTLYLQMNSLR |
| | | AEDTAVYYCARRDYWYSVYTHRYARTFDVWGQGTLVTVSS |
| 84 | CXCR5-1-51 | EVQLVESGGGLVQPGGSLRLSCAASGFTFSNYWMGWVRQAPGK |
| | | GLEWVSVISYDGSITYYPDSVKGRFTISRDNSKNTLYLQMNSLRA |
| | | EDTAVYYCARDRHGNYAFDYWGQGTLVTVSS |
| 85 | CXCR5-1-52 | EVQLVQSGAEVKKPGSSVKVSCKASGGTFSSYYMSWVRQAPGQ |
| | | GLEWIGRIRPYDGYTHYNQKFKGRVTITRDTSTSTVYMELSSLRS |
| | | EDTAVYYCARRGYSRDWFAYWGQGTLVTVSS |
| 86 | CXCR5-1-53 | EVQLVQSGAEVKKPGSSVKVSCKASGGTFSDYWMSWVRQAPG |
| | | QGLEWIGRIRPGDGETYYAQKFKGRVTITRDTSTSTVYMELSSLR |
| | | SEDTAVYYCARLFFSSDDFAFAFDYWGQGTLVTVSS |
| 87 | CXCR5-1-54 | EVQLVESGGGLVQPGGSLRLSCAASGFTFSDYWMGWVRQAPGK |
| | | GLEWVAVISYSGSNTYYPDSVKGRFTISRDNSKNTLYLQMNSLR |
| | | AEDTAVYYCARDLTGYYPFDYWGQGTLVTVSS |
| 88 | CXCR5-1-55 | EVQLVESGGGLVQPGGSLRLSCAASGFTFSDYAMSWVRQAPGK |
| | | GLEWVAYISPSGSNKYYPDSVKGRFTISRDNSKNTLYLQMNSLR |
| | | AEDTAVYYCARDDDGYLDYLRFNFDYWGQGTLVTVSS |
| 89 | CXCR5-1-56 | EVQLVQSGAEVKKPGSSVKVSCKASGGTFSNYWMNWVRQAPG |
| | | QGLEWIGVIRPNNGETHYNQKFKGRVTITRDTSTSTVYMELSSLR |
| | | SEDTAVYYCARLYGPNTVTYYMDYWGQGTLVTVSS |
| 90 | CXCR5-1-57 | EVQLVQSGAEVKKPGSSVKVSCKASGGTFSSYAMHWVRQAPGQ |
| | | GLEWIGRINPNNGETKYAQKFKGRVTITRDTSTSTVYMELSSLRS |
| | | EDTAVYYCARGSAYYHYYYYSHGGAFAYWGQGTLVTVSS |
| 91 | CXCR5-1-58 | EVQLVESGGGLVQPGGSLRLSCAASGFTFSSYYMHWVRQAPGK |
| | | GLEWVAYISPDGGNKYYPDSVKGRFTISRDNSKNTLYLQMNSLR |
| | | AEDTAVYYCARRVHWYGRYTHNYYYGLDVWGQGTLVTVSS |
| 92 | CXCR5-1-59 | EVQLVQSGAEVKKPGSPVKVSCKASGGTFSSYWMNWVRQAPG |
| | | QGLEWIGVINPGDGYTKYNQKFKGRVTITRDTSTSTVYMELSSL |
| | | RSEDTAVYYCARHESGYGVGAYGFAYWGQGTLVTVSS |
| 93 | CXCR5-1-60 | EVQLVQSGAEVKKPGSSVKVSCKASGGTFSDYAMHWVRQAPG |
| | | QGLEWIGVINPYNGYTKYADKFKGRVTITRDTSTSTVYMELSSL |
| | | RSEDTAVYYCARPGEPYDTYITSFGFQMDYWGQGTLVTVSS |
| 94 | CXCR5-1-61 | EVQLVESGGGLVQPGGSLRLSCAASGFTFSNYAMSWVRQAPGK |
| | | GLEWVAVISYDGSEKYYADSVKGRFTISRDNSKNTLYLQMNSLR |
| | | AEDTAVYYCARGRSDYYDLHTHNFDYWGQGTLVTVSS |
| 95 | CXCR5-1-62 | EVQLVQSGAEVKKPGSSVKVSCKASGGTFSSYWMHWVRQAPG |
| | | QGLEWIGRINPGDGYTYYNDKFKGRVTITRDTSTSTVYMELSSL |
| | | RSEDTAVYYCARLESKYDVGSAMDYWGQGTLVTVSS |
| 96 | CXCR5-1-63 | EVQLVESGGGLVQPGGSLRLSCAASGFTFSSYGMSWVRQAPGK |
| | | GLEWVSYISPSGGNTYYADSVKGRFTISRDNSKNTLYLQMNSLR |
| | | AEDTAVYYCARISVRYIRTGNDYARTMDYWGQGTLVTVSS |
| 97 | CXCR5-1-64 | EVQLVESGGGLVQPGGSLRLSCAASGFTFSSYAMGWVRQAPGK |
| | | GLEWVAVISYSGSNKYYADSVKGRFTISRDNSKNTLYLQMNSLR |
| | | AEDTAVYYCARIDHWDGRWGYYHARTMDVWGQGTLVTVSS |
| 98 | CXCR5-1-65 | EVQLVESGGGLVQPGGSLRLSCAASGFTFSSYGMHWVRQAPGK |
| | | GLEWVSVISPNGGETYYPDSVKGRFTISRDNSKNTLYLQMNSLR |
| | | AEDTAVYYCARGTSRYLPLHTYYFDYWGQGTLVTVSS |
| 99 | CXCR5-1-66 | EVQLVESGGGLVQPGGSLRLSCAASGFTFSDYWMHWVRQAPGK |
| | | GLEWVSVISYSGGETYYPDSVKGRFTISRDNSKNTLYLQMNSLR |
| | | AEDTAVYYCARIRTYNYPFDYWGQGTLVTVSS |
| 100 | CXCR5-1-67 | EVQLVQSGAEVKKPGSSVKVSCKASGGTFSNYAMHLVRQAPGQ |
| | | GLEWIGTINPYNGYTYYADKFKGRVTITRDTSTSTVYMELSSLRS |
| | | EDTAVYYCARHYLWYYYFAAMDYWGQGTLVTVSS |
| 101 | CXCR5-1-68 | EVQLVESGGGLVQPGGSLRLSCAASGFTFSSYWMSWVRQAPGK |
| | | GLEWVSVISYSGSNKYYADSVKGRFTISRDNSKNTLYLQMNSLR |
| | | AEDTAVYYCARIDTDNFAFDYWGQGTLVTVSS |
| 102 | CXCR5-1-69 | EVQLVESGGGLVQPGGSLRLSCAASGFTFSDYYMHWVRQAPGK |
| | | GLEWVSYISPDGGIKYYPDSVKGRFTISRDNSKNTLYLQMNSLR |
| | | AEDTAVYYCARDEDYYGIFYGQNHYFGFGMDVWGQGTLVTVS |
| | | S |
| 103 | CXCR5-1-70 | EVQLVQSGAEVKKPGSSVKVSCKASGGTFSSYAMNWVRQAPGQ |
| | | GLEWIGVIRPNNGYTHYNDKFKGRVTITRDTSTSTVYMELSSLRS |
| | | EDTAVYYCARPSAYIDVSYTSFYGYFAYWGQGTLVTVSS |
| 104 | CXCR5-1-71 | EVQLVESGGGLVQPGGSLRLSCAASGFTFSSYWMHWVRQAPGK |
| | | GLEWVSYISPSGGNTYYPDSVKGRFTISRDNSKNTLYLQMNSLR |
| | | AEDTAVYYCARDRDYNFAFDYWSQGTLVTVSS |
| 105 | CXCR5-1-72 | EVQLVESGGGLVQPGGSLRLSCAASGFTFSSYWMSWVRQAPGK |
| | | GLEWVSYISPDGSNKYYADSVKGRFTISRDNSKNTLYLQMNSLR |
| | | AEDTAVYYCARDRLHYGDSWRYNHHKYGGMDVWGQGTLVTV |
| | | SS |
| 106 | CXCR5-1-73 | EVQLVESGGGLVQPGGSLRLSCAASGFTFSNYGMSWVRQAPGK |
| | | GLEWVSYISYDGGNKYYPDSVKGRFTISRDNSKNTLYLQMNSLR |
| | | AEDTAVYYCARIRDYGYGFDYWGQGTLVTVSS |
| 107 | CXCR5-1-74 | EVQLVQSGAEVKKPGSSVKVSCKASGGTFSSYAMNWVRQAPGQ |
| | | GLEWIGVIRPYDGYTHYNDKFKGRVTITRDTSTSTVYMELSSLRS |
| | | EDTAVYYCARSYYKHNNLAYMDYWGQGTLVTVSS |
| 108 | CXCR5-1-75 | EVQLVQSGAEVKKPGSSVKVSCKASGGTFSSYAMNWVRQAPGQ |
| | | GLEWIGRIRPGNGETHYNQKFKGRVTITRDTSTSTVYMELSSLRS |
| | | EDTAVYYCARHLSKYFVTNAMDYWGQGTLVTVSS |
| 109 | CXCR5-1-76 | EVQLVESGGGLVQPGGSLRLSCAASGFTFSNYAMSWVRQAPGK |
| | | GLEWVAVISPDGGIKYYADSVKGRFTISRDNSKNTLYLQMNSLR |
| | | AEDTAVYYCARIVGRDDRSGNDYYRTMDYWGQGTLVTVSS |
| 110 | CXCR5-1-77 | EVQLVESGGGLVQPGGSLRLSCAASGFTFSNYYMHWVRQAPGK |
| | | GLEWVSVISYSGGEKYYPDSVKGRFTISRDNSKNTLYLQMNSLR |
| | | AEDTAVYYCARIDHDNYGFDYWGQGTLVTVSS |
| 111 | CXCR5-1-78 | EVQLVQSGAEVKKPGSSVKVSCKASGGTFSSYYMHWVRQAPGQ |
| | | GLEWIGVIRPYNGYTKYNQKFKGRVTITRDTSTSTVYMELSSLRS |
| | | EDTAVYYCARDFFGNYVYSFWFDYWGQGTLVTVSS |
| 112 | CXCR5-1-79 | EVQLVESGGGLVQPGGSLRLSCAASGFTFSSYYMSWVRQAPGK |
| | | GLEWVSYISYDGGETYYADSVKGRFTISRDNSKNTLYLQMNSLR |
| | | AEDTAVYYCARDELYYYIGWGHDHHFHRGMDVWGQGTLVTV |
| | | SS |
| 113 | CXCR5-1-80 | EVQLVESGGGLVQPGGSLRLSCAASGFTFSDYYMHWVRQAPGK |
| | | GLEWVSVISYSGSEKYYPDSVKGRFTISRDNSKNTLYLQMNSLR |
| | | AEDTAVYYCARGDRGYYSFWTHPFDYWGQGTLVTVSS |
| 114 | CXCR5-1-81 | EVQLVESGGGLVQPGGSLRLSCAASGFTFSDYGMSWVRQAPGK |
| | | GLEWVAYISPDGGEKYYPDSVKGRFTISRDNSKNTLYLQMNSLR |
| | | AEDTAVYYCARDRTNGFGFDYWGQGTLVTVSS |
| 115 | CXCR5-1-82 | EVQLVESGGGLVQPGGSLRLSCAASGFTFSDYWMHWVRQAPGK |
| | | GLEWVAYISPDGGNKYYPDSVKGRFTISRDNSKNTLYLQMNSLR |
| | | AEDTAVYYCARGELHRGSSTRYDFHYYRGMDVWGQGTLVTVS |
| | | S |
| 116 | CXCR5-1-83 | EVQLVESGGGLVQPGGSLRLSCAASGFTFSDYGMHWVRQAPGK |
| | | GLEWVAYISYSGGITYYPDSVKGRFTISRDNSKNTLYLQMNSLR |
| | | AEDTEVYYCARPSYYDSLWRHRYYRTFDVWGQGTLVTVSS |
| 117 | CXCR5-1-84 | EVQLVESGGGLVQPGGSLRLSCAASGFTFSSYAMGWVRQAPGK |
| | | GLEWVAVISPDGSITYYPDSVKGRFTISRDNSKNTLYLQMNSLRA |
| | | EDTAVYYCARHRTDNFPFDYWGQGTLVTVSS |
| 118 | CXCR5-1-85 | EVQLVQSGAEVKKPGSSVKVSCKASGGTFSNYWMSWVRQAPG |
| | | QGLEWIGRINPYNGETYYNDKFKGRVTITRDTSTSTVYMELSSLR |
| | | SEDTAVYYCARSPFGFTYYSTYFAYWGQGTLVTVSS |
| 119 | CXCR5-1-86 | EVQLVESGGGLVQPGGSLRLSCAASGFTFSNYAMHWVRQAPGK |
| | | GLEWVSVISPSGGITYYADSVKGRFTISRDNSKNTLYLQMNSLRA |
| | | EDTAVYYCARPRYLFGRTGNRYYYTLDVWGQGTLVTVSS |
| 120 | CXCR5-1-87 | EVQLVQSGAEVKKPGSSVKVSCKASGGTFSSYWMNWVRQAPG |
| | | QGLEWIGVIRPYDGYTHYNDKFKGRVTITRDTSTSTVYMELSSL |
| | | RSEDTAVYYCARHYSDYTDTSYMDYWGQGTLVTVSS |
| 121 | CXCR5-1-88 | EVQLVESGGGLVQPGGSLRLSCAASGFTFSDYYMHWVRQAPGK |
| | | GLEWVSYISPDGGITYYPDSVKGRFTISRDNSKNTLYLQMNSLRA |
| | | EDTAVYYCARDDTNNDPFDYWGQGTLVTVSS |
| 122 | CXCR5-1-89 | EVQLVESGGGLVQPGGSLRLSCAASGFTFSSYGMSWVRQAPGK |
| | | GLEWVAYISYSGSEKYYADSVKGRFTISRDNSKNTLYLQMNSLR |
| | | AEDTAVYYCARGEGHYYDSTRQRFYFYFPMDVWGQGTLVTVS |
| | | S |
| 123 | CXCR5-1-90 | EVQLVESGGGLVQPGGSLRLSCAASGFTFSDYAMGWVRQAPGK |
| | | GLEWVAVISPSGSNKYYADSVKGRFTISRDNSKNTLYLQMNSLR |
| | | AEDTAVYYCARRRYRFGFWRQHHAYTFDVWGQGTLVTVSS |
| 124 | CXCR5-1-91 | EVQLVQSGAEVKKPGSSVKVSCKASGGTFSNYWMNWVRQAPG |
| | | QGLEWIGVIRPGNGETKYADKFKGRVTITRDTSTSTVYMELSSLR |
| | | SEDTAVYYCARSYLSSYDLYAMDYWGQGTLVTVSS |
| 125 | CXCR5-1-92 | EVQLVESGGGLVQPGGSLRLSCAASGFTFSNYWMHWVRQAPGK |
| | | GLEWVSVISPSGSEKYYPDSVKGRFTISRDNSKNTLYLQMNSLRA |
| | | EDTAVYYCARDKDSNGILHGQNFDYWGQGTLVTVSS |
| 126 | CXCR5-1-93 | EVQLVQSGAEVKKPGSSVKVSCKASGGTFSNYYMSWVRQAPGQ |
| | | GLEWIGRIRPGDGYTYYADKFKGRVTITRDTSTSTVYMELSSLRS |
| | | EDTAVYYCARGYNWARKLVYWGQGTLVTVSS |
| 127 | CXCR5-1-94 | EVQLVESGGGLVQPGGSLRLSCAASGFTFSSYWMSWVRQAPGK |
| | | GLEWVSVISYDGSEKYYPDSVKGRFTISRDNSKNTLYLQMNSLR |
| | | AEDTAVYYCARGKSGWYPLHGQNFDYWGQGTLVTVSS |
| 128 | CXCR5-1-95 | EVQLVQSGAEVKKPGSSVKVSCKASGGTFSSYWMNWVRQAPG |
| | | QGLEWIGVIRPNNGYTHYADKFKGRVTITRDTSTSTVYMELSSL |
| | | RSEDTAVYYCARHFIYYGGFSTGFDYWGQGTLVTVSS |
| 129 | CXCR5-1-96 | EVQLVESGGGLVQPGGSLRLSCAASGFTFSSYYMSWVRQAPGK |
| | | GLEWVSVISPNGGEKYYPDSVKGRFTISRDNSKNTLYLQMNSLR |
| | | AEDTAVYYCARGDQDNGGRLGYYFDYWGQGTLVTVSS |
| 130 | CXCR5-1-97 | EVQLVESGGGLVQPGGSLRLSCAASGFTFSNYWMHWVRQAPGK |
| | | GLEWVAVISYSGGITYYPDSVKGRFTISRDNSKNTLYLQMNSLR |
| | | AEDTAVYYCARDRTNYFPFDYWGQGTLVTVSS |
| 131 | CXCR5-1-98 | EVQLVESGGGLVQPGGSLRLSCAASGFTFSDYYMSWVRQAPGK |
| | | GLEWVSVISPSGGITYYPDSVKGRFTISRDNSKNTLYLQMNSLRA |
| | | EDTAVYYCARISHYVGLWRHYYYRGFDVWGQGTLVTVSS |
| 132 | CXCR5-1-99 | EVQLVQSGAEVKKPGSSVKVSCKASGGTFSDYYMSWVRQAPGQ |
| | | GLEWIGTIRPNNGETKYNQKFKGRVTITRDTSTSTVYMELSSLRS |
| | | EDTAVYYCARLTSRSTDGQFAFDYWGQGTLVTVSS |
| 133 | CXCR5-1-100 | EVQLVESGGGLVQPGGSLRLSCAASGFTFSDYAMGWVRQAPGK |
| | | GLEWVAYISYSGSNTYYPDSVKGRFTISRDNSKNTLYLQMNSLR |
| | | AEDTAVYYCARISVYFDLWGYYHYYGLDYWGQGTLVTVSS |
| 134 | CXCR5-1-101 | EVQLVQSGAEVKKPGSSVKVSCKASGGTFSSYWMNWVRQAPG |
| | | QGLEWIGTIRPNDGETKYNDKFKGRVTITRDTSTSTVYMELSSLR |
| | | SEDTAVYYCARLTFRFTNGYGGFDYWGQGTLVTVSS |
| 135 | CXCR5-1-102 | EVQLVESGGGLVQPGGSLRLSCAASGFTFSNYAMGWVRQAPGK |
| | | GLEWVSYISPSGSIKYYPDSVKGRFTISRDNSKNTLYLQMNSLRA |
| | | EDTAVYYCARGRGYYYIGTGHRGHKHRPMDVWGQGTLVTVSS |
| 136 | CXCR5-1-103 | EVQLVESGGGLVQPGGSLRLSCAASGFTFSDYWMHWVRQAPGK |
| | | GLEWVSVISPNGGITYYPDSVKGRFTISRDNSKNTLYLQMNSLRA |
| | | EDTAVYYCARDTDSRLPYHRQPFDYWGQGTLVTVSS |
| 137 | CXCR5-1-104 | EVQLVQSGAEVKKPGSSVKVSCKASGGTFSNYYMHWVRQAPG |
| | | QGLEWIGTIRPNNGYTKYNDKFKGRVTITRDTSTSTVYMELSSLR |
| | | SEDTAVYYCARLYYSSYNLAAMDYWGQGTLVTVSS |
| 138 | CXCR5-1-105 | EVQLVQSGAEVKKPGSSVKVSCKASGGTFSSYWMSWVRQAPG |
| | | QGLEWIGTINPGDGYTKYNDKFKGRVTITRDTSTSTVYMELSSLR |
| | | SEDTAVYYCARFYYYFDKLVYWGQGTLVTVSS |
| 139 | CXCR5-1-106 | EVQLVESGGGLVQPGGSLRLSCAASGFTFSSYGMHWVRQAPGK |
| | | GLEWVAYITYYPDSVKGRFTISRDNSKNTLYLQMNSLRAEDTAV |
| | | YYCARGDDGNFPFDYWGQGTLVTVSS |
| 140 | CXCR5-1-107 | EVQLVQSGAEVKKPGSSVKVSCKASGGTFSSYAMNWVRQAPGQ |
| | | GLEWIGRIRPNNGYNDKFKGRVTITRDTSTSTVYMELSSLRSEDT |
| | | AVYYCARPGEYMDYEITYAPFQFAYWGQGTLVTVSS |
| 141 | CXCR5-1-108 | EVQLVQSGAEVKKPGSSVKVSCKASGGTFSDYAMHWVRQAPG |
| | | QGLEWIGRIRPGDGYTHYADKFKGRVTITRDTSTSTVYMELSSLR |
| | | SEDTAVYYCARFGHSGRSFAYWGQGTLVTVSS |
| 142 | CXCR5-1-109 | EVQLVQSGAEVKKPGSSVKVSCKASGGTFSNYWMNWVRQAPG |
| | | QGLEWIGRINPGNGETHYADKFKGRVTITRDTSTSTVYMELSSLR |
| | | SEDTAVYYCARDPIDSYYFAYGFDYWGQGTLVTVSS |
| 143 | CXCR5-1-110 | EVQLVQSGAEVKKPGSSVKVSCKASGGTFSDYAMNWVRQAPG |
| | | QGLEWIGRINPNDGETYYNQKFKGRVTITRDTSTSTVYMELSSLR |
| | | SEDTAVYYCARHGAPMSVSYTSHPFQMDYWGQGTLVTVSS |
| 144 | CXCR5-1-111 | EVQLVQSGAEVKKPGSSVKVSCKASGGTFSDYAMSWVRQAPGQ |
| | | GLEWIGRIRPGNGYTHYNDKFKGRVTITRDTSTSTVYMELSSLRS |
| | | EDTAVYYCARFYYYGAWLDYWGQGTLVTVSS |
| 145 | CXCR5-1-112 | EVQLVESGGGLVQPGGSLRLSCAASGFTFSSYYMSLVRQAPGKG |
| | | LEWVSVISYDGSEKYYADSVKGRFTISRDNSKNTLYLQMNSLRA |
| | | EDTAVYYCARPSHYYDLWTQYYAYGLDYWGQGTLVTVSS |
| 146 | CXCR5-1-113 | EVQLVQSGAEVKKPGSSVKVSCKASGGTFSNYAMNWVRQAPG |
| | | QGLEWIGRIRPNNGETHYNQKFKGRVTITRDTSTSTVYMELSSLR |
| | | SEDTAVYYCARHTISYGYSQTWFDYWGQGTLVTVSS |
| 147 | CXCR5-1-114 | EVQLVQSGAEVKKPGSSVKVSCKASGGTFSNYWMNWVRQAPG |
| | | QGLEWIGVINPYDGYTHYADKFKGRVTITRDTSTSTVYMELSSL |
| | | RSEDTAVYYCARLTGYFDVFAYGFDYWGQGTLVTVSS |
| 148 | CXCR5-1-115 | EVQLVESGGGLVQPGGSLRLSCAASGFTFWVRQAPGKGLEWVS |
| | | YISYDGGSIKYYPDSVKGRFTISRDNSKNTLYLQMNSLRAEDTAV |
| | | YYCARDRGYYYDGTTYNFGKGFPMDVWGQGTLVTVSS |
| 149 | CXCR5-1-116 | EVQLVQSGAEVKKPGSSVKVSCKASGGTFSDYYMSWVRQAPGQ |
| | | GLEWIGTINPYDGYTYYADKFKGRVTITRDTSTSTVYMELSSLRS |
| | | EDTAVYYCARLSFGNDYFQYAFDYWGQGTLVTVSS |
| 150 | CXCR5-1-117 | EVQLVESGGGLVQPGGSLRLSCAASGFTFSSYWMHWVRQAPGK |
| | | GLEWVSYISPDGSNKYYPDSVKGRFTISRDNSKNTLYLQMNSLR |
| | | AEDTAVYYCARKRHYDIFYGQRGARTFDVWGQGTLVTVSS |
| 151 | CXCR5-1-118 | EVQLVESGGGLVQPGGSLRLSCAASGFTFSSYYMHWVRQAPGK |
| | | GLEWVSVISPNGGIKYYADSVKGRFTISRDNSKNTLYLQMNSLR |
| | | AEDTAVYYCARDKSDYGIYWTQGFDYWGQGTLVTVSS |
| 152 | CXCR5-1-119 | EVQLVQSGAEVKKPGSSVKVSCKASGGTFSSYWMSWVRQAPG |
| | | QGLEWIGRIRPNNGYTKYNQKFKGRVTITRDTSTSTVYMELSSLR |
| | | SEDTAVYYCARSFSSNGGYSGAFAYWGQGTLVTVSS |
| 153 | CXCR5-1-120 | EVQLVESGGGLVQPGGSLRLSCAASGFTFSDYWMHWVRQAPGK |
| | | GLEWVSVISYDGGEKYYPDSVKGRFTISRDNSKNTLYLQMNSLR |
| | | AEDTAVYYCARHLDYGYGFDYWGQGTLVTVSS |
| 154 | CXCR5-1-121 | EVQLVESGGGLVQPGGSLRLSCAASGFTFSSYYMGWVRQAPGK |
| | | GLEWVSYISYNGGNTYYPDSVKGRFTISRDNSKNTLYLQMNSLR |
| | | AEDTAVYYCARDRDNYYSSTGQYFHKGRPMDVWGQGTLVTVS |
| | | S |
| 155 | CXCR5-1-122 | EVQLVESGGGLVQPGGSLRLSCAASGFTFSNYAMGLVRQAPGK |
| | | GLEWVSYISYSGSETYYADSVKGRFTISRDNSKNTLYLQMNSLR |
| | | AEDTAVYYCARGTDSYGDFYTFNFDYWGQGTLVTVSS |
| 156 | CXCR5-1-123 | EVQLVESGGGLVQPGGSLRLSCAASGFTFSNYGMHWVRQAPGK |
| | | GLEWVAYISYSGGNKYYADSVKGRFTISRDNSKNTLYLQMNSL |
| | | RAEDTAVYYCARPDVRDILWRYYYYRGMDYWGQGTLVTVSS |
| 157 | CXCR5-1-124 | EVQLVESGGGLVQPGGSLRLSCAASGFTFSNYWMSWVRQAPGK |
| | | GLEWVAYISYDGSNTYYPDSVKGRFTISRDNSKNTLYLQMNSLR |
| | | AEDTAVYYCARDEGHYYDFYTHDGGYYGGMDVWGQGTLVTV |
| | | SS |
| 158 | CXCR5-1-125 | EVQLVESGGGLVQPGGSLRLSCAASGFTFSDYAMHWVRQAPGK |
| | | GLEWVSVISYDGGITYYADSVKGRFTISRDNSKNTLYLQMNSLR |
| | | AEDTAVYYCARGEDYRYSFYGYYYYKYFPMDVWGQGTLVTVS |
| | | S |
| 159 | CXCR5-1-126 | EVQLVESGGGLVQPGGSLRLSCAASGFTFSSYYMSWVRQAPGK |
| | | GLEWVSYADSVKGRFTISRDNSKNTLYLQMNSLRAEDTAVYYC |
| | | ARATSRWGPYYRQGFDYWGQGTLVTVSS |
| 160 | CXCR5-1-127 | EVQLVESGGGLVQPGGSLRLSCAASGFTFSNYYMHWVRQAPGK |
| | | GLEWVSYISPSGGEKYYADSVKGRFTISRDNSKNTLYLQMNSLR |
| | | AEDTAVYYCARPRGLYSVYTNDHARGLDYWGQGTLVTVSS |
| 161 | CXCR5-1-128 | EVQLVESGGGLVQPGGSLRLSCAASGFTFSSYAMSWVRQAPGK |
| | | GLEWVAVISYNGGNTYYPDSVKGRFTISRDNSKNTLYLQMNSLR |
| | | AEDTAVYYCARGDDNNYAFDYWGQGTLVTVSS |
| 162 | CXCR5-1-129 | EVQLVESGGGLVQPGGSLRLSCAASGFTFSNYAMHWVRQAPGK |
| | | GLEWVAYISYSGGNTYYPDSVKGRFTISRDNSKNTLYLQMNSLR |
| | | AEDTAVYYCARDDRNGFPFDYWGQGTLVTVSS |
| 163 | CXCR5-1-130 | EVQLVESGGGLVQPGGSLRLSCAASGFTFSSYAMSWVRQAPGK |
| | | GLEWVAVISPNGGNKYYPDSVKGRFTISRDNSKNTLYLQMNSLR |
| | | AEDTAVYYCARGLHNWYAFDYWGQGTLVTVSS |
| 164 | CXCR5-1-131 | EVQLVESGGGLVQPGGSLRLSCAASGFTFSNYGMHWVRQAPGK |
| | | GLEWVSVISYSGGITYYADSVKGRFTISRDNSKNTLYLQMNSLR |
| | | AEDTAVYYCARIRDNYFPFDYWGQGTLVTVSS |
| 165 | CXCR5-1-132 | EVQLVESGGGLVQPGGSLRLSCAASGFTFSNYWMHWVRQAPGK |
| | | GLEWVAYISYDGSNTYYPDSVKGRFTISRDNSKNTLYLQMNSLR |
| | | AEDTAVYYCARIRHLFGFSTQDHARGFDVWGQGTLVTVSS |
| 166 | CXCR5-1-133 | EVQLVESGGGLVQPGGSLRLSCAASGFTFSDYGMSWVRQAPGK |
| | | GLEWVSVISYNGGNKYYPDSVKGRFTISRDNSKNTLYLQMNSLR |
| | | AEDTAVYYCARDELYRGSGWGYYGYYGYPMDVWGQGTLVTV |
| | | SS |
| 167 | CXCR5-1-134 | EVQLVESGGGLVQPGGSLRLSCAASGFTFSNYWMSWVRQAPGK |
| | | GLEWVSVISPNGGITYYADSVKGRFTISRDNSKNTLYLQMNSLR |
| | | AEDTAVYYCARHDDNNFGFDYWGQGTLVTVSS |
| 168 | CXCR5-1-135 | EVQLVQSGAEVKKPGSSVKVSCKASGGTFSSYWMSWVRQAPG |
| | | QGLEWIGTIRPGNGETYYNQKFKGRVTITRDTSTSTVYMELSSLR |
| | | SEDTAVYYCARGYSYAAYLDYWGQGTLVTVSS |
| 169 | CXCR5-1-136 | EVQLVESGGGLVQPGGSLRLSCAASGFTFSNYGMSWVRQAPGK |
| | | GLEWVAVISPSGGIKYYPDSVKGRFTISRDNSKNTLYLQMNSLR |
| | | AEDTAVYYCARIRHGNYAFDYWGQGTLVTVSS |
| 170 | CXCR5-1-137 | EVQLVESGGGLVQPGGSLRLSCAASGFTFSNYAMSWDRQAPGK |
| | | GLEWVSYISYNGGITYYPDSVKGRFTISRDNSKNTLYLQMNSLR |
| | | AEDTAVYYCARGRRGNDPFDYWGQGTLVTVSS |
| 171 | CXCR5-1-138 | EVQLVQSGAEVKKPGSSVKVSCKASGGTFSSYWMNWVRQAPG |
| | | QGLEWIGVINPNDGYTKYAQKFKGRVTITRDTSTSTVYMELSSL |
| | | RSEDTAVYYCARLFISYDDFNTAFDYWGQGTLVTVSS |
| 172 | CXCR5-1-139 | EVQLVESGGGLVQPGGSLRLSCAASGFTFSDYYMSWVRQAPGK |
| | | GLEWVSVISYDGSNTYYPDSVKGRFTISRDNSKNTLYLQMNSLR |
| | | AEDTAVYYCARGTQRRTDLHTYPFDYWGQGTLVTVSS |
| 173 | CXCR5-1-140 | EVQLVESGGGLVQPGGSLRLSCAASGFTFSSYWMHWVRQAPGK |
| | | GLEWVAYISPSGSETYYPDSVKGRFTISRDNSKNTLYLQMNSLR |
| | | AEDTAVYYCARDPSRWTGWYRYPFDYWGQGTLVTVSS |
| 174 | CXCR5-1-141 | EVQLVESGGGLVQPGGSLRLSCAASGFTFSDYGMGWVRQAPGK |
| | | GLEWVSYISPSGSEKYYPDSVKGRFTISRDNSKNTLYLQMNSLRA |
| | | EDTAVYYCARIDRDYFAFDYWGQGTLVTVSS |
| 175 | CXCR5-1-142 | EVQLVQSGAEVKKPGSSVKVSCKASGGTFSNYWMSWVRQAPG |
| | | QGLEWIGTIRPNDGETKYNDKFKGRVTITRDTSTSTVYMELSSLR |
| | | SEDTAVYYCARSTSYYYNYATWFAYWGQGTLVTVSS |
| 176 | CXCR5-1-143 | EVQLVQSGAEVKKPGSSVKVSCKASGGTFSSYAMSWVRQAPGQ |
| | | GLEWIGVIRPNNGYTHYADKFKGRVTITRDTSTSTVYMELSSLRS |
| | | EDTAVYYCARDYYWYFVYSAIDYWGQGTLVTVSS |
| 177 | CXCR5-1-144 | EVQLVESGGGLVQPGGSLRLSCAASGFTFSSYYMHWVRQAPGK |
| | | GLEWVSVISPDGGETYYPDSVKGRFTISRDNSKNTLYLQMNSLR |
| | | AEDTAVYYCARDRDDRGILWTYNFDYWGQGTLVTVSS |
| 178 | CXCR5-1-145 | EVQLVESGGGLVQPGGSLRLSCAASGFTFSDYWMHWVRQAPGK |
| | | GLEWVAYISYDGGITYYADSVKGRFTISRDNSKNTLYLQMNSLR |
| | | AEDTAVYYCARIFVLFSLTGQNYYRTLDYWGQGTLVTVSS |
| 179 | CXCR5-1-146 | EVQLVESGGGLVQPGGSLRLSCAASGFTFSNYGMHWVRQAPGK |
| | | GLEWVAYISYDGGNKYYPDSVKGRFTISRDNSKNTLYLQMNSL |
| | | RAEDTAVYYCARDDSDWTSLLRFNFDYWGQGTLVTVSS |
| 180 | CXCR5-1-147 | EVQLVESGGGLVQPGGSLRLSCAASGFTFSNYWMDWVRQAPGK |
| | | GLEWVSVISYDGSNTYYPDSVKGRFTISRDNSKNTLYLQMNSLR |
| | | AEDTAVYYCARHDRDGYAFDYWGQGTLVTVSS |
| 181 | CXCR5-1-148 | EVQLVQSGAEVKKPGSSVKVSCKASGGTFSNYAMNWVRQAPG |
| | | QGLEWIGVIRPGNGYTYYNQKFKGRVTITRDTSTSTVYMELSSL |
| | | RSEDTAVYYCARLTSRFYNFQYYFAYWGQGTLVTVSS |
| 182 | CXCR5-1-149 | EVQLVESGGGLVQPGGSLRLSCAASGFTFSDYAMHWVRQAPGK |
| | | GLEWVSYISYSGSNKYYADSVKGRFTISRDNSKNTLYLQMNSLR |
| | | AEDTAVYYCARGELYYYSGSYYDYGYYYGMDVWGQGTLVTV |
| | | SS |
| 183 | CXCR5-1-150 | EVQLVQSGAEVKKPGSSVKVSCKASGGTFSSYYMSWVRQAPGQ |
| | | GLEWIGVIRPNDGETYYAQKFKGRVTITRDTSTSTVYMELSSLRS |
| | | EDTAVYYCARDEYSYTYGYYMDYWGQGTLVTVSS |
| 184 | CXCR5-1-151 | EVQLVESGGGLVQPGGSLRLSCAASGFTFSSYAMGWVRQAPGK |
| | | GLEWVAVISYSGSNKYYADSVKGRFTISRDNSKNTLYLQMNSLR |
| | | AEDTAVYYCARHLHDNFAFDYWGQGTLVTVSS |
| 185 | CXCR5-1-152 | EVQLVESGGGLVQPGGSLRLSCAASGFTFSSYWMSWVRQAPGK |
| | | GLEWVAYISPDGGIKYYPDSVKGRFTISRDNSKNTLYLQMNSLR |
| | | AEDTAVYYCARRVVLFDLTGYDYAYTFDYWGQGTLVTVSS |
| 186 | CXCR5-1-153 | EVQLVESGGGLVQPGGSLRLSCAASGFTFSSYWMGWVRQAPGK |
| | | GLEWVAYISYDGGNTYYADSVKGRFTISRDNSKNTLYLQMNSL |
| | | RAEDTAVYYCARGEDNRYISSGYDYYYHGPMDVWGQGTLVTV |
| | | SS |
| 187 | CXCR5-1-154 | EVQLVQSGAEVKKPGSSVKVSCKASGGTFSNYWMSWVRQAPG |
| | | QGLEWIGTIRPNNGETHYNQKFKGRVTITRDTSTSTVYMELSSLR |
| | | SEDTAVYYCARHSRPYDTSYTYFGFAMDYWGQGTLVTVSS |
| 188 | CXCR5-1-155 | EVQLVQSGAEVKKPGSSVKVSCKASGGTFSNYYMNWVRQAPG |
| | | QGLEWIGRIRLNNGYTKYNQKFKGRVTITRDTSTSTVYMELSSL |
| | | RSEDTAVYYCARLPFGSGYSSTAFDYWGHGTLVTVSS |
| 189 | CXCR5-1-156 | EVQLVQSGAEVKKPGSSVKVSCKASGGTFSSYYMSWVRQAPGQ |
| | | GLEWIGTIRPNDGYTKYNDKFKGRVTITRDTSTSTVYMELSSLRS |
| | | EDTAVYYCARHSEPSDVSITSFPYTFDYWGQGTLVTVSS |
| 190 | CXCR5-1-157 | EVQLVQSGAEVKKPGSSVKVSCKASGGTFSSYAMNWVRQAPGQ |
| | | GLEWIGRINPNDGYTYYAQKFKGRVTITRDTSTSTVYMELSSLRS |
| | | EDTAVYYCARHGSPNTYYYYMDYWGQGTLVTVSS |
| 191 | CXCR5-1-158 | EVQLVQSGAEVKKPGSSVKVSCKASGGTFSNYWMSWVRQAPG |
| | | QGLEWIGTIRPNNGETKYNDKFKGRVTITRDTSTSTVYMELSSLR |
| | | SEDTAVYYCARGYGSGAAFDYWGQGTLVTVSS |
| 192 | CXCR5-1-159 | EVQLVESGGGLVQPGGSLRLSCAASGFTFSDYWMGWVRQAPGK |
| | | GLEWVSVISPSGSETYYPDSVKGRFTISRDNSKNTLYLQMNSLRA |
| | | EDTAVYYCARDEDHYYIFWGHNYHYHRPMDVWGQGTLVTVSS |
| 193 | CXCR5-1-160 | EVQLVQSGAEVKKPGSSVKVSCKASGGTFSNYWMSWVRQAPG |
| | | QGLEWIGVINPGDGYTKYNQKFKGRVTITRDTSTSTVYMELSSL |
| | | RSEDTAVYYCARDYSWHDYLNYMDYWGQGTLVTVSS |
| 194 | CXCR5-1-161 | EVQLVESGGGLVQPGGSLRLSCAASGFTFSNYAMSWVRQAPGK |
| | | GLEWVSVISPNGGNKYYPDSVKGRFTISRDNSKNTLYLQMNSLR |
| | | AEDTAVYYCARDEGHYYSGWTFNHHKYGGMDVWGQGTLVTV |
| | | SS |
| 195 | CXCR5-1-162 | EVQLVESGGGLVQPGGSLRLSCAASGFTFSNYGMSWVRQAPGK |
| | | GLEWVSYISYSGGNTYYPDSVKGRFTISRDNSKNTLYLQMNSLR |
| | | AEDTAVYYCARIDVWDSFWGYDHARGLDVWGQGTLVTVSS |
| 196 | CXCR5-1-163 | EVQLVQSGAEVKKPGSSVKVSCKASGGTFSDYAMNWVRQAPG |
| | | QGLEWIGRIRPGDGETHYNQKFKGRVTITRDTSTSTVYMELSSLR |
| | | SEDTAVYYCARHFGRFTVFQGGFAYWGQGTLVTVSS |
| 197 | CXCR5-1-164 | EVQLVQSGAEVKKPGSSVKVSCKASGGTFSDYAMHWVRQAPG |
| | | QGLEWIGTINPGDGYTKYADKFKGRVTITRDTSTSTVYMELSSLR |
| | | SEDTAVYYCARLYSSNFGYSAMDYWGQGTLVTVSS |
| 198 | CXCR5-1-165 | EVQLVESGGGLVQPGGSLRLSCAASGFTFSSYGMHWVRQAPGK |
| | | GLEWVSVISYNGGEKYYADSVKGRFTISRDNSKNTLYLQMNSLR |
| | | AEDTAVYYCARDEGHRGDSLRFDFHKHFPMDVWGQGTLVTVS |
| | | S |
| 199 | CXCR5-2-1 | EVQLVESGGGLVQPGGSLRLSCAASGSTISDRAMGWFRQAPGKE |
| | | REMVAAIIGDATNYADSVKGRFTISADNSKNTAYLQMNSLKPED |
| | | TAVYYCARALQYCSPTSCYVDDYFYYMDVWGQGTLVTVSS |
| 200 | CXCR5-2-2 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCARGPEWTPPGDYFYYMDDWGQGTLVTVSS |
| 201 | CXCR5-2-3 | EVQLVESGGGLVQPGGSLRLSCAASGFSLDDYGMGWFRQAPGK |
| | | EREGVAAIGSDGSTSYADSVKGHFTISADNSKNTAYLQMNSLKP |
| | | EDTAVYYCGTWFGDYNFWGQGTLVTVSS |
| 202 | CXCR5-2-4 | EVQLVESGGGLVQPGGSLRLSCAASGRGFSRYAMGWFRQAPGK |
| | | EREFVAAITPINWGGRGTTVYADSVKGRFTISADNSKNTAYLQM |
| | | NSLKPEDTAVYYCASDPPGWGQGTLVTVSS |
| 203 | CXCR5-2-5 | EVQLVESGGGLVQPGGSLRLSCAASGNIAAINVMGWFRQAPGKE |
| | | REFVAAISWSSGSTAYADSVKGRFTISADNSKNTAYLQMNSLKP |
| | | EDTAVYYCVRDRGGLWGQGTLVTVSS |
| 204 | CXCR5-2-6 | EVQLVESGGGLVQPGGSLRLSCAASDLSFSFYTMGWFRQAPGKE |
| | | RELVATINWSGTPVYADSVKGRFTISADNSKNTAYLQMNSLKPE |
| | | DTAVYYCAREDDYYDGTGYYQYYGMDVWGQGTLVTVSS |
| 205 | CXCR5-2-7 | EVQLVESGGGLVQPGGSLRLSCAASGFTVSNYAMGWFRQAPGK |
| | | EREFVAAIRWSGGITWYADSVKGRFTISADNSKNTAYLQMNSLK |
| | | PEDTAVYYCVRDRGGSWGQGTLVTVSS |
| 206 | CXCR5-2-8 | EVQLVESGGGLVQPGGSLRLSCAASGFTLDYYAMGWFRQAPGK |
| | | ERELVAAINWSGDTIYYADSVKGRFTISADNSKNTAYLQMNSLK |
| | | PEDTAVYYCAREGCSSTSCYLDPWGQGTLVTVSS |
| 207 | CXCR5-2-9 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCVLTLSPYAMDVWGQGTLVTVSS |
| 208 | CXCR5-2-10 | EVQLVESGGGLVQPGGSLRLSCAASGFSFDDDYVMGWFRQAPG |
| | | KEREFVSAIDWNGNSTYYADSVKGRFTISADNSKNTAYLQMNSL |
| | | KPEDTAVYYCARGPEWTPPGDYFYYMDDWGQGTLVTVSS |
| 209 | CXCR5-2-11 | EVQLVESGGGLVQPGGSLRLSCAASGGTFSIYAMGWFRQAPGKE |
| | | REFVAAISTHSITVYADSVKGRFTISADNSKNTAYLQMNSLKPED |
| | | TAVYYCATYLEMSPGEYFDNWGQGTLVTVSS |
| 210 | CXCR5-2-12 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCASYWRTGDWFDPWGQGTLVTVSS |
| 211 | CXCR5-2-13 | EVQLVESGGGLVQPGGSLRLSCAASGITFRRYIMGWFRQAPGKE |
| | | REFVAAISSSGALTSYADSVKGRFTISADNSKNTAYLQMNSLKPE |
| | | DTAVYYCAKDRTGSGWFRDVWGQGTLVTVSS |
| 212 | CXCR5-2-14 | EVQLVESGGGLVQPGGSLRLSCAASGIPSIRAMGWFRQAPGKER |
| | | ELVAGISRSGETTWYADSVKGRFTISADNSKNTAYLQMNSLKPE |
| | | DTAVYYCVKSGLDDGYYPEDWGQGTLVTVSS |
| 213 | CXCR5-2-15 | EVQLVESGGGLVQPGGSLRLSCAASGSIDSIHVMGWFRQAPGKE |
| | | REFVAAISWTGGSTAYADSVKGRFTISADNSKNTAYLQMNSLKP |
| | | EDTAVYYCASDPPGWGQGTLVTVSS |
| 214 | CXCR5-2-16 | EVQLVESGGGLVQPGGSLRLSCAASGSTISDRAMGWFRQAPGKE |
| | | REMVAAIIGDATNYADSVKGRFTISADNSKNTAYLQMNSLKPED |
| | | TAVYYCTTDMGGWGQGTLVTVSS |
| 215 | CXCR5-2-17 | EVQLVESGGGLVQPGGSLRLSCAASGMTTIGPMGWFRQAPGKE |
| | | REMVAAISWSGGLTYYADSVKGRFTISADNSKNTAYLQMNSLK |
| | | PEDTAVYYCARVYYDSSGYNDYWGQGTLVTVSS |
| 216 | CXCR5-2-18 | EVQLVESGGGLVQPGGSLRLSCAASGSTISDRAMGWFRQAPGKE |
| | | REMVAAIIGDATNYADSVKGRFTISADNSKNTAYLQMNSLKPED |
| | | TAVYYCARGPEWTPPGDYFYYMDDWGQGTLVTVSS |
| 217 | CXCR5-2-19 | EVQLVESGGGLVQPGGSLRLSCAASGSIDSIHVMGWFRQAPGKE |
| | | REFVAAISWTGGSTAYADSVKGRFTISADNSKNTAYLQMNSLKP |
| | | EDTAVYYCVAGMVRGVDFWGQGTLVTVSS |
| 218 | CXCR5-2-20 | EVQLVESGGGLVQPGGSLRLSCAASGRTFSDYAMGWFRQAPGK |
| | | EREFVAVVNWNGDSTYYADSVKGRFTISADNSKNTAYLQMNSL |
| | | KPEDTAVYYCARLFAQYSDYDYVAEWGQGTLVTVSS |
| 219 | CXCR5-2-21 | EVQLVESGGGLVQPGGSLRLSCAASGRTFFSYPMGWFRQAPGKE |
| | | REFVAAIRWSGGSTYYADSVKGRFTISADNSKNTAYLQMNSLKP |
| | | EDTAVYYCASGRPVPRWGQGTLVTVSS |
| 220 | CXCR5-2-22 | EVQLVESGGGLVQPGGSLRLSCAASGNIFRIETMGWFRQAPGKE |
| | | REFVATIHSSGSTYYADSVKGRFTISADNSKNTAYLQMNSLKPED |
| | | TAVYYCARSDYDVVSGLTNDYLYYLDDWGQGTLVTVSS |
| 221 | CXCR5-2-23 | EVQLVESGGGLVQPGGSLRLSCAASGFNFDDYAMGWFRQAPGK |
| | | EREWVSEISSGGNKDYADSVKGRFTISADNSKNTAYLQMNSLKP |
| | | EDTAVYYCARTSYYYSSGSSFSGRLDYLDDWGQGTLVTVSS |
| 222 | CXCR5-2-24 | EVQLVESGGGLVQPGGSLRLSCAASGFPFSEYPMGWFRQAPGKE |
| | | RELVAGIAWGDGITYYADSVKGRFTISADNSKNTAYLQMNSLKP |
| | | EDTAVYYCITIFVGMDVWGQGTLVTVSS |
| 223 | CXCR5-2-25 | EVQLVESGGGLVQPGGSLRLSCAASGFPFDDYAMGWFRQAPGK |
| | | ERELVAAITRSGKTTYYADSVKGRFTISADNSKNTAYLQMNSLK |
| | | PEDTAVYYCARVYYDSSGYNDYWGQGTLVTVSS |
| 224 | CXCR5-2-26 | EVQLVESGGGLVQPGGSLRLSCAASGFPFDDYAMGWFRQAPGK |
| | | EREFVAAISWSAGSTYYADSVKGRFTISADNSKNTAYLQMNSLK |
| | | PEDTAVYYCARVRDFWGGYDIDHWGQGTLVTVSS |
| 225 | CXCR5-2-27 | EVQLVESGGGLVQPGGSLRLSCAASGFNLDDYADMGWFRQAPG |
| | | KEREFVAAVTWSGGLTSYADSVKGRFTISADNSKNTAYLQMNS |
| | | LKPEDTAVYYCVRDRGGSWGQGTLVTVSS |
| 226 | CXCR5-2-28 | EVQLVESGGGLVQPGGSLRLSCAASGFGIDAMGWFRQAPGKER |
| | | EFVAAISWSGDSTYYADSVKGRFTISADNSKNTAYLQMNSLKPE |
| | | DTAVYYCARAGGPYYDLSTGSSGHLDYWGQGTLVTVSS |
| 227 | CXCR5-2-29 | EVQLVESGGGLVQPGGSLRLSCAASGFDFDNFDDYAMGWFRQA |
| | | PGKEREFVAAINRSGDTTYYADSVKGRFTISADNSKNTAYLQMN |
| | | SLKPEDTAVYYCAKAGPNYYDSDTRGDYWGQGTLVTVSS |
| 228 | CXCR5-2-30 | EVQLVESGGGLVQPGGSLRLSCAASGFNFDDYAMGWFRQAPGK |
| | | EREVVASISTDVDSKYYADSVKGRFTISADNSKNTAYLQMNSLK |
| | | PEDTAVYYCARAEGYWYFDLWGQGTLVTVSS |
| 229 | CXCR5-2-31 | EVQLVESGGGLVQPGGSLRLSCAASGFGFGSYDMGWFRQAPGK |
| | | EREGVSCFTSSDGRTFYADSVKGRFTISADNSKNTAYLQMNSLK |
| | | PEDTAVYYCARAPYTSVAGRAYYYYYGMDVWGQGTLVTVSS |
| 230 | CXCR5-2-32 | EVQLVESGGGLVQPGGSLRLSCAASGFDFDNFDDYAMGWFRQA |
| | | PGKEREFVAAINRSGDTTYYADSVKGRFTISADNSKNTAYLQMN |
| | | SLKPEDTAVYYCGTWFGDYNFWGQGTLVTVSS |
| 231 | CXCR5-2-33 | EVQLVESGGGLVQPGGSLRLSCAASGFPFSIWPMGWFRQAPGKE |
| | | REFVAAIRWSGASTVYADSVKGRFTISADNSKNTAYLQMNSLKP |
| | | EDTAVYYCARLDILGGPDTVGAFDLWGQGTLVTVSS |
| 232 | CXCR5-2-34 | EVQLVESGGGLVQPGGSLRLSCAASGFPFSEYPMGWFRQAPGKE |
| | | RELVAGIAWGDGITYYADSVKGRFTISADNSKNTAYLQMNSLKP |
| | | EDTAVYYCTTDMGGWGQGTLVTVSS |
| 233 | CXCR5-2-35 | EVQLVESGGGLVQPGGSLRLSCAASGFSFDDYAMGWFRQAPGK |
| | | ERELVAAVRWSGGITWYADSVKGRFTISADNSKNTAYLQMNSL |
| | | KPEDTAVYYCVRVARDRGYNYDSDWGQGTLVTVSS |
| 234 | CXCR5-2-36 | EVQLVESGGGLVQPGGSLRLSCAASGFPLDDYAMGWFRQAPGK |
| | | ERELVAGIAWGDGSTYYADSVKGRFTISADNSKNTAYLQMNSL |
| | | KPEDTAVYYCARTFKTGYRSGYYWGQGTLVTVSS |
| 235 | CXCR5-2-37 | EVQLVESGGGLVQPGGSLRLSCAASGFPLDDYAMGWFRQAPGK |
| | | ERELVAGISSEGTTYYADSVKGRFTISADNSKNTAYLQMNSLKPE |
| | | DTAVYYCVTDQSAYGQTVFFDSWGQGTLVTVSS |
| 236 | CXCR5-2-38 | EVQLVESGGGLVQPGGSLRLSCAASGFPLDYYGMGWFRQAPGK |
| | | ERELVAAISRSGGSTYYADSVKGRFTISADNSKNTAYLQMNSLK |
| | | PEDTAVYYCARDPDDYGDYTFDYWGQGTLVTVSS |
| 237 | CXCR5-2-39 | EVQLVESGGGLVQPGGSLRLSCAASGFSFDDDYVMGWFRQAPG |
| | | KEREFVAAISRSGGDTFYADSVKGRFTISADNSKNTAYLQMNSL |
| | | KPEDTAVYYCVAGMVRGVDFWGQGTLVTVSS |
| 238 | CXCR5-2-40 | EVQLVESGGGLVQPGGSLRLSCAASGFSFDDDYVMGWFRQAPG |
| | | KEREFVSAIDWNGNSTYYADSVKGRFTISADNSKNTAYLQMNSL |
| | | KPEDTAVYYCAGWIHMKGGFLDYWGQGTLVTVSS |
| 239 | CXCR5-2-41 | EVQLVESGGGLVQPGGSLRLSCAASGFSFDDDYVMGWFRQAPG |
| | | KEREFVSAIDWNGNSTYYADSVKGRFTISADNSKNTAYLQMNSL |
| | | KPEDTAVYYCARADCSGGVCNAYWGQGTLVTVSS |
| 240 | CXCR5-2-42 | EVQLVESGGGLVQPGGSLRLSCAASGFAFSRYGMGWFRQAPGK |
| | | ERELVAGITPGGNTNYADSVKGRFTISADNSKNTAYLQMNSLKP |
| | | EDTAVYYCAMTSWGLVYWGQGTLVTVSS |
| 241 | CXCR5-2-43 | EVQLVESGGGLVQPGGSLRLSCAASGFSFDDYAMGWFRQAPGK |
| | | ERELVSDISFGGNTNYADSVKGRFTISADNSKNTAYLQMNSLKPE |
| | | DTAVYYCARTSYYYSSGSSFSGRLDYLDDWGQGTLVTVSS |
| 242 | CXCR5-2-44 | EVQLVESGGGLVQPGGSLRLSCAASGFSLDDYGMGWFRQAPGK |
| | | EREGVAAIGSDGSTSYADSVKGHFTISADNSKNTAYLQMNSLKP |
| | | EDTAVYYCARALQYCSPTSCYVDDYFYYMDVWGQGTLVTVSS |
| 243 | CXCR5-2-45 | EVQLVESGGGLVQPGGSLRLSCAASGFSLDDYGMGWFRQAPGK |
| | | EREGVAAIGSDGSTSYADSVKGRFTISADNSKNTAYLQMNSLKP |
| | | EDTAVYYCARGFSSGWYGWDSWGQGTLVTVSS |
| 244 | CXCR5-2-46 | EVQLVESGGGLVQPGGSLRLSCAASGFSLDDYGMGWFRQAPGK |
| | | ERELVAAISRSGNVTAYADSVKGHFTISADNSKNTAYLQMNSLK |
| | | PEDTAVYYCGTWFGDYNFWGQGTLVTVSS |
| 245 | CXCR5-2-47 | EVQLVESGGGLVQPGGSLRLSCAASGFSLDDYGMGWFRQAPGK |
| | | EREFVAGVAWSSDFTAYADSVKGRFTISADNSKNTAYLQMNSL |
| | | KPEDTAVYYCARASPGRYCSGRSCYFDWYFHLWGQGTLVTVSS |
| 246 | CXCR5-2-48 | EVQLVESGGGLVQPGGSLRLSCAASGFSLDYYAMGWFRQAPGK |
| | | EREFVASISWIIGSTYYADSVKGRFTISADNSKNTAYLQMNSLKP |
| | | EDTAVYYCARALQYCSPTSCYVDDYFYYMDVWGQGTLVTVSS |
| 247 | CXCR5-2-49 | EVQLVESGGGLVQPGGSLRLSCAASGFSLDYYAMGWFRQAPGK |
| | | EREFVASISWIIGSTYYADSVKGRFTISADNSKNTAYLQMNSLKP |
| | | EDTAVYYCARVNPSDYYDSRGYPDYWGQGTLVTVSS |
| 248 | CXCR5-2-50 | EVQLVESGGGLVQPGGSLRLSCAASGFAFSTASMGWFRQAPGKE |
| | | REFVAAITRGSTYYADSVKGRFTISADNSKNTAYLQMNSLKPED |
| | | TAVYYCVLTLSPYAMDVWGQGTLVTVSS |
| 249 | CXCR5-2-51 | EVQLVESGGGLVQPGGSLRLSCAASGDTFNWYAMGWFRQAPG |
| | | KERELVATITADGITNYADSVKGRFTISADNSKNTAYLQMNSLKP |
| | | EDTAVYYCARDREAYSYGYNDYWGQGTLVTVSS |
| 250 | CXCR5-2-52 | EVQLVESGGGLVQPGGSLRLSCAASGFAFDDYAMGWFRQAPGK |
| | | EREIVAAIRWSGGITWYADSVKGRFTISADNSKNTAYLQMNSLK |
| | | PEDTAVYYCATEETLQQLLRAYCWGQGTLVTVSS |
| 251 | CXCR5-2-53 | EVQLVESGGGLVQPGGSLRLSCAASGFTDDYYAMGWFRQAPGK |
| | | EREFVAAISWSGGSTYYADSVKGRFTISADNSKNTAYLQMNSLK |
| | | PEDTAVYYCARGPYGGASYFTVWGQGTLVTVSS |
| 252 | CXCR5-2-54 | EVQLVESGGGLVQPGGSLRLSCAASGFTFENYAMGWFRQAPGK |
| | | EREFVAAINWNGASTDYADSVKGRFTISADNSKNTAYLQMNSL |
| | | KPEDTAVYYCARDHPNYYYGMDVWGQGTLVTVSS |
| 253 | CXCR5-2-55 | EVQLVESGGGLVQPGGSLRLSCAASGFTFSTHWMGWFRQAPGK |
| | | ERELLAEIYPSGSYYADSVKGRFTISADNSKNTAYLQMNSLKPED |
| | | TAVYYCAREGPRVDLNYDFWSPDYYYYMDVWGQGTLVTVSS |
| 254 | CXCR5-2-56 | EVQLVESGGGLVQPGGSLRLSCAASDLSFSFYTMGWFRQAPGKE |
| | | RELVAAVTSGGITNYADSVKGRFTISADNSKNTAYLQMNSLKPE |
| | | DTAVYYCAREDDYYDGTGYYQYYGMDVWGQGTLVTVSS |
| 255 | CXCR5-2-57 | EVQLVESGGGLVQPGGSLRLSCAASGRGFSRYAMGWFRQAPGK |
| | | EREFVAAITPINWGGRGTTVYADSVKGRFTISADNSKNTAYLQM |
| | | NSLKPEDTAVYYCAREDDYYDGTGYYQYYGMDVWGQGTLVT |
| | | VSS |
| 256 | CXCR5-2-58 | EVQLVESGGGLVQPGGSLRLSCAASGSTFSKAVMGWFRQAPGK |
| | | EREFVAAISSSGISTIYADSVKGRFTISADNSKNTAYLQMNSLKPE |
| | | DTAVYYCARGGGPHYYYYYYMDVWGQGTLVTVSS |
| 257 | CXCR5-2-59 | EVQLVESGGGLVQPGGSLRLSCAASGSTFSSYRMGWFRQAPGKE |
| | | REFVSAINYSGGSTYYADSVKGRFTISADNSKNTAYLQMNSLKP |
| | | EDTAVYYCAREGEYSSSWYYYYYGMDVWGQGTLVTVSS |
| 258 | CXCR5-2-60 | EVQLVESGGGLVQPGGSLRLSCAASGYFASWYYMGWFRQAPG |
| | | KERELVAGVSRGGMTSLGDSTLYADSVKGRFTISADNSKNTAYL |
| | | QMNSLKPEDTAVYYCARDRPDYYYYYGMDVWGQGTLVTVSS |
| 259 | CXCR5-2-61 | EVQLVESGGGLVQPGGSLRLSCAASGCTVSINAMGWFRQAPGK |
| | | EREFVAAISWSGGSTYYADSVKGRFTISADNSKNTAYLQMNSLK |
| | | PEDTAVYYCARLFAQYSDYDYVAEWGQGTLVTVSS |
| 260 | CXCR5-2-62 | EVQLVESGGGLVQPGGSLRLSCAASGDIFSNYGMGWFRQAPGK |
| | | EREGVAAIGSDGSTSYADSVKGRFTISADNSKNTAYLQMNSLKP |
| | | EDTAVYYCATAVGATSDDPFDMWGQGTLVTVSS |
| 261 | CXCR5-2-63 | EVQLVESGGGLVQPGGSLRLSCAASGDIGSINAMGWFRQAPGKE |
| | | RELVAAIRWSGGITWYADSVKGRFTISADNSKNTAYLQMNSLKP |
| | | EDTAVYYCVKSGGNYGDYVVWGQGTLVTVSS |
| 262 | CXCR5-2-64 | EVQLVESGGGLVQPGGSLRLSCAASGDIGSINAMGWFRQAPGKE |
| | | REFVAAITPINWGGRGTTVYADSVKGRFTISADNSKNTAYLQMN |
| | | SLKPEDTAVYYCVMRGSGVATRVYWGQGTLVTVSS |
| 263 | CXCR5-2-65 | EVQLVESGGGLVQPGGSLRLSCAASGDIGSINAMGWFRQAPGKE |
| | | REFVAAVRWSGGITWYADSVKGRFTISADNSKNTAYLQMNSLK |
| | | PEDTAVYYCARTRHDYSNVYWGQGTLVTVSS |
| 264 | CXCR5-2-66 | EVQLVESGGGLVQPGGSLRLSCAASGDIGSINAMGWFRQAPGKE |
| | | RELVAGISRSGGTTYYADSVKGRFTISADNSKNTAYLQMNSLKP |
| | | EDTAVYYCLAVTSGADAFDIWGQGTLVTVSS |
| 265 | CXCR5-2-67 | EVQLVESGGGLVQPGGSLRLSCAASGDISSIVAMGWFRQAPGKE |
| | | RELVAAIRWSEDRVWYADSVKGRFTISADNSKNTAYLQMNSLK |
| | | PEDTAVYYCARDQGREDDFWSGYDEPRDVWGQGTLVTVSS |
| 266 | CXCR5-2-68 | EVQLVESGGGLVQPGGSLRLSCAASGDISSIVAMGWFRQAPGKE |
| | | RELVAAIRWSEDRVWYADSVKGRFTISADNSKNTAYLQMNSLK |
| | | PEDTAVYYCARTFKTGYRSGYYWGQGTLVTVSS |
| 267 | CXCR5-2-69 | EVQLVESGGGLVQPGGSLRLSCAASGDTFNWYAMGWFRQAPG |
| | | KEREIVAAIDWSGSSTYYADSVKGRFTISADNSKNTAYLQMNSL |
| | | KPEDTAVYYCANLEFNYYDSRQLRWGQGTLVTVSS |
| 268 | CXCR5-2-70 | EVQLVESGGGLVQPGGSLRLSCAASGDTFNWYAMGWFRQAPG |
| | | KEREIVAAISRSGDTTYYADSVKGRFTISADNSKNTAYLQMNSLK |
| | | PEDTAVYYCARASSDYGDVSGPWGQGTLVTVSS |
| 269 | CXCR5-2-71 | EVQLVESGGGLVQPGGSLRLSCAASGDTFNWYAMGWFRQAPG |
| | | KEREIVAAISRSGDTTYYADSVKGRFTISADNSKNTAYLQMNSLK |
| | | PEDTAVYYCARTGSSSPDSYMDVWGQGTLVTVSS |
| 270 | CXCR5-2-72 | EVQLVESGGGLVQPGGSLRLSCAASGDTFNWYAMGWFRQAPG |
| | | KEREFVAAISRSGSITYYADSVKGRFTISADNSKNTAYLQMNSLK |
| | | PEDTAVYYCVSDVGNNWYADSWGQGTLVTVSS |
| 271 | CXCR5-2-73 | EVQLVESGGGLVQPGGSLRLSCAASGDTFNWYAMGWFRQAPG |
| | | KEREFVAAISWSEDNTYYADSVKGRFTISADNSKNTAYLQMNSL |
| | | KPEDTAVYYCVKAAQDYGDSTFDFWGQGTLVTVSS |
| 272 | CXCR5-2-74 | EVQLVESGGGLVQPGGSLRLSCAASGDTFNWYAMGWFRQAPG |
| | | KERELVASITNGGSTSYADSVKGRFTISADNSKNTAYLQMNSLKP |
| | | EDTAVYYCVGCSGGSCNYWGQGTLVTVSS |
| 273 | CXCR5-2-75 | EVQLVESGGGLVQPGGSLRLSCAASGDTFSSYSMGWFRQAPGKE |
| | | REFVAAVTWSGGITWYADSVKGRFTISADNSKNTAYLQMNSLK |
| | | PEDTAVYYCARDLYYDSSGYYGGWGQGTLVTVSS |
| 274 | CXCR5-2-76 | EVQLVESGGGLVQPGGSLRLSCAASGDTFSWYAMGWFRQAPGK |
| | | EREFVAAISNSGLSTYYADSVKGRFTISADNSKNTAYLQMNSLKP |
| | | EDTAVYYCARAYCSGGSCYDYWGQGTLVTVSS |
| 275 | CXCR5-2-77 | EVQLVESGGGLVQPGGSLRLSCAASGDTFSWYAMGWFRQAPGK |
| | | EREFQAAISRSGGTTYYADSVKGRFTISADNSKNTAYLQMNSLK |
| | | PEDTAVYYCARVMESGYDYLDYWGQGTLVTVSS |
| 276 | CXCR5-2-78 | EVQLVESGGGLVQPGGSLRLSCAASGDTFSWYAMGWFRQAPGK |
| | | EREFVAAISSSGEVTTYADSVKGRFTISADNSKNTAYLQMNSLKP |
| | | EDTAVYYCARIVLVAVGELTDYWGQGTLVTVSS |
| 277 | CXCR5-2-79 | EVQLVESGGGLVQPGGSLRLSCAASGERAFSNYAMGWFRQAPG |
| | | KERELVAAVTSGGTTYYADSVKGRFTISADNSKNTAYLQMNSL |
| | | KPEDTAVYYCARGPEWTPPGDYFYYMDDWGQGTLVTVSS |
| 278 | CXCR5-2-80 | EVQLVESGGGLVQPGGSLRLSCAASGFSLDYYGMGWFRQAPGK |
| | | EREFVAAIDWSGGTTYYADSVKGRFTISADNSKNTAYLQMNSLK |
| | | PEDTAVYYCARGPEWTPPGDYFYYMDDWGQGTLVTVSS |
| 279 | CXCR5-2-81 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCARDWQSLVRGVSIDQWGQGTLVTVSS |
| 280 | CXCR5-2-82 | EVQLVESGGGLVQPGGSLRLSCAASGFTDDYYAMGWFRQAPGK |
| | | ERELVAGIDTSGIVNYADSVKGRFTISADNSKNTAYLQMNSLKPE |
| | | DTAVYYCARGQLRYFDWLLDYYFDYWGQGTLVTVSS |
| 281 | CXCR5-2-83 | EVQLVESGGGLVQPGGSLRLSCAASGFTFSSYPMGWFRQAPGKE |
| | | REFVAAISSSGVTTIYADSVKGRFTISADNSKNTAYLQMNSLKPE |
| | | DTAVYYCASDPPGWGQGTLVTVSS |
| 282 | CXCR5-2-84 | EVQLVESGGGLVQPGGSLRLSCAASGFTFSTSWMGWFRQAPGK |
| | | ERELVALISMSGDDSAYADSVKGRFTISADNSKNTAYLQMNSLK |
| | | PEDTAVYYCARGNYYMDVWGQGTLVTVSS |
| 283 | CXCR5-2-85 | EVQLVESGGGLVQPGGSLRLSCAASGFTFSTYWMGWFRQAPGK |
| | | ERELVAAINWDSARTYYADSVKGRFTISADNSKNTAYLQMNSL |
| | | KPEDTAVYYCTTDQHWGQGTLVTVSS |
| 284 | CXCR5-2-86 | EVQLVESGGGLVQPGGSLRLSCAASGFTFSTYWMGWFRQAPGK |
| | | ERELVAAISGGGSITNYADSVKGRFTISADNSKNTAYLQMNSLKP |
| | | EDTAVYYCARGPEWTPPGDYFYYMDDWGQGTLVTVSS |
| 285 | CXCR5-2-87 | EVQLVESGGGLVQPGGSLRLSCAASGFTFSTYWMGWFRQAPGK |
| | | EREMVAAISGSGATNYADSVKGRFTISADNSKNTAYLQMNSLKP |
| | | EDTAVYYCARGPEWTPPGDYFYYMDVWGQGTLVTVSS |
| 286 | CXCR5-2-88 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCAGGSYGGYVWGQGTLVTVSS |
| 287 | CXCR5-2-89 | QVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCAHQYCAAGSCYDKWGQGTLVTVSS |
| 288 | CXCR5-2-90 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCAKAERGSERAYWGQGTLVTVSS |
| 289 | CXCR5-2-91 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCAKAGPNYYDSDTRGDYWGQGTLVTVSS |
| 290 | CXCR5-2-92 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCARDGDFWSGYRDYWGQGTLVTVSS |
| 291 | CXCR5-2-93 | EVQLVESGGGLVQPGGSLRLSCAASGSIYSLDAMGWFRQAPGKE |
| | | REFVAAISRSGSITYYADSVKGRFTISADNSKNTAYLQMNSLKPE |
| | | DTAVYYCTTDHYVWGTFDPWGQGTLVTVSS |
| 292 | CXCR5-2-94 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCARGPYGGASYFTVWGQGTLVTVSS |
| 293 | CXCR5-2-95 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCARGVGYCGGMGCHEGDYWGQGTLVTVSS |
| 294 | CXCR5-2-96 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCARPYCSSTSCYSSWGQGTLVTVSS |
| 295 | CXCR5-2-97 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCARQMCGGGDCYIHWGQGTLVTVSS |
| 296 | CXCR5-2-98 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCARVYYDSSGYYDYWGQGTLVTVSS |
| 297 | CXCR5-2-99 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCASLWAGYDGDYFNYWGQGTLVTVSS |
| 298 | CXCR5-2-100 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCATSKLVGSTYVDYWGQGTLVTVSS |
| 299 | CXCR5-2-101 | EVQLVESGGGLVQPGGSLRLSCAASGFTFSTYWMGWFRQAPGK |
| | | ERELVAAISGSGSITNYADSVKGRFTISADNSKNTAYLQMNSLKP |
| | | EDTAVYYCATWMGTYGDDYWGQGTLVTVSS |
| 300 | CXCR5-2-102 | EVQLVESGGGLVQPGGSLRLSCAASGFTFSTYWMGWFRQAPGK |
| | | EREFVAATSSSGGSTSYADSVKGRFTISADNSKNTAYLQMNSLKP |
| | | EDTAVYYCARAGYEDYWGQGTLVTVSS |
| 301 | CXCR5-2-103 | EVQLVESGGGLVQPGGSLRLSCAASGFTFSTYWMGWFRQAPGK |
| | | EREFVAATSSSGGSTSYADSVKGRFTISADNSKNTAYLQMNSLKP |
| | | EDTAVYYCARGPEWTPPGDYFYYMDDWGQGTLVTVSS |
| 302 | CXCR5-2-104 | EVQLVESGGGLVQPGGSLRLSCAASGFTFSTYWMGWFRQAPGK |
| | | ERELVAHIYSDGSINYADSVKGRFTISADNSKNTAYLQMNSLKPE |
| | | DTAVYYCAKVESEDLLVDSLIYWGQGTLVTVSS |
| 303 | CXCR5-2-105 | EVQLVESGGGLVQPGGSLRLSCAASGFTFSTYWMGWFRQAPGK |
| | | EREFVAVVNWNGDSTYYADSVKGRFTISADNSKNTAYLQMNSL |
| | | KPEDTAVYYCARGPEWTPPGDYFYYMDDWGQGTLVTVSS |
| 304 | CXCR5-2-106 | EVQLVESGGGLVQPGGSLRLSCAASGFTIDDYAMGWFRQAPGK |
| | | ERELVSLINSDGTTSYADSVKGRFTISADNSKNTAYLQMNSLKPE |
| | | DTAVYYCARVVYGSDSFDDFWGQGTLVTVSS |
| 305 | CXCR5-2-107 | EVQLVESGGGLVQPGGSLRLSCAASGFTLDAYAMGWFRQAPGK |
| | | EREFVAAINSGGSTEYADSVKGRFTISADNSKNTAYLQMNSLKPE |
| | | DTAVYYCANAYDFWSGPVYWGQGTLVTVSS |
| 306 | CXCR5-2-108 | EVQLVESGGGLVQPGGSLRLSCAASGFTLDAYAMGWFRQAPGK |
| | | EREFVAAINSGGSTEYADSVKGRFTISADNSKNTAYLQMNSLKPE |
| | | DTAVYYCVRPNLRYTYGYDYWGQGTLVTVSS |
| 307 | CXCR5-2-109 | EVQLVESGGGLVQPGGSLRLSCAASGFTLDAYAMGWFRQAPGK |
| | | EREFVAAISKSDGSTYYADSVKGRFTISADNSKNTAYLQMNSLK |
| | | PEDTAVYYCARVYYDSSGYNDYWGQGTLVTVSS |
| 308 | CXCR5-2-110 | EVQLVESGGGLVQPGGSLRLSCAASGFTLDAYAMGWFRQAPGK |
| | | ERELVAAISRSGNTYYADSVKGRFTISADNSKNTAYLQMNSLKP |
| | | EDTAVYYCARNRLTGDSSQVFWGQGTLVTVSS |
| 309 | CXCR5-2-111 | EVQLVESGGGLVQPGGSLRLSCAASGFTFSSYPMGWFRQAPGKE |
| | | REFVAAISSSGVTTYYADSVKGRFTISADNSKNTAYLQMNSLKPE |
| | | DTAVYYCVTDQSAYGQTVFFDSWGQGTLVTVSS |
| 310 | CXCR5-2-112 | EVQLVESGGGLVQPGGSLRLSCAASGFTFSSYPMGWFRQAPGKE |
| | | REFVAAISSSGVTTIYADSVKGRFTISADNSKNTAYLQMNSLKPE |
| | | DTAVYYCARGPYYYDSSGYYGPNDYWGQGTLVTVSS |
| 311 | CXCR5-2-113 | EVQLVESGGGLVQPGGSLRLSCAASGFTDDYYVMGWFRQAPGK |
| | | EREFVAVISWSGSNTYYADSVKGRFTISADNSKNTAYLQMNSLK |
| | | PEDTAVYYCARALQYCSPTSCYVDDYFYYMDVWGQGTLVTVSS |
| 312 | CXCR5-2-114 | EVQLVESGGGLVQPGGSLRLSCAASGFTDGIDAMGWFRQAPGK |
| | | ERELVAVISWSGGITWYADSVKGRFTISADNSKNTAYLQMNSLK |
| | | PEDTAVYYCVRAGDTRNDYNYGAYWGQGTLVTVSS |
| 313 | CXCR5-2-115 | EVQLVESGGGLVQPGGSLRLSCAASGFTFDDTGMGWFRQAPGK |
| | | EREGVAAIGSDGSTSYADSVKGRFTISADNSKNTAYLQMNSLKP |
| | | EDTAVYYCAKGAQWEQRTYDSWGQGTLVTVSS |
| 314 | CXCR5-2-116 | EVQLVESGGGLVQPGGSLRLSCAASGFTFDDYNMGWFRQAPGK |
| | | EREGVSYISSSDGSTYYADSVKGRFTISADNSKNTAYLQMNSLKP |
| | | EDTAVYYCAAALDGYSGSWGQGTLVTVSS |
| 315 | CXCR5-2-117 | EVQLVESGGGLVQPGGSLRLSCAASGFTFDDYPMGWFRQAPGK |
| | | ERELVAAIRWSDGTTYYADSVKGRFTISADNSKNTAYLQMNSLK |
| | | PEDTAVYYCARLVVPANTYFYYAMDVWGQGTLVTVSS |
| 316 | CXCR5-2-118 | EVQLVESGGGLVQPGGSLRLSCAASGFTFDDYPMGWFRQAPGK |
| | | ERELVAAIRWSDGTTYYADSVKGRFTISADNSKNTAYLQMNSLK |
| | | PEDTAVYYCATDGADTAPIYGMAVWGQGTLVTVSS |
| 317 | CXCR5-2-119 | EVQLVESGGGLVQPGGSLRLSCAASGFTFDDYVMGWFRQAPGK |
| | | EREFVAAISRSPGVTYYADSVKGRFTISADNSKNTAYLQMNSLKP |
| | | EDTAVYYCAREPGPADYRDYWGQGTLVTVSS |
| 318 | CXCR5-2-120 | EVQLVESGGGLVQPGGSLRLSCAASGFTFDDYVMGWFRQAPGK |
| | | EREFVAAISTGGDTTYYADSVKGRFTISADNSKNTAYLQMNSLK |
| | | PEDTAVYYCATDLSGRGDVSEYEYDWGQGTLVTVSS |
| 319 | CXCR5-2-121 | EVQLVESGGGLVQPGGSLRLSCAASGFTFDDYVMGWFRQAPGK |
| | | EREGVSWISSSDKDTYYADSVKGRFTISADNSKNTAYLQMNSLK |
| | | PEDTAVYYCVKVANDYGNYEPSWGQGTLVTVSS |
| 320 | CXCR5-2-122 | EVQLVESGGGLVQPGGSLRLSCAASGFTFDGYAMGWFRQAPGK |
| | | ERELVAAVSWDGRNTYYADSVKGRFTISADNSKNTAYLQMNSL |
| | | KPEDTAVYYCVRAGDTRNDYNYGAYWGQGTLVTVSS |
| 321 | CXCR5-2-123 | EVQLVESGGGLVQPGGSLRLSCAASGFTFDRSWMGWFRQAPGK |
| | | EREWVAGIGSDGTTIYADSVKGRFTISADNSKNTAYLQMNSLKP |
| | | EDTAVYYCARDYYDSSGYYYVWGQGTLVTVSS |
| 322 | CXCR5-2-124 | EVQLVESGGGLVQPGGSLRLSCAASGFTFDRSYHMGWFRQAPG |
| | | KEREFVTAINWSLTRTHYADSVKGRFTISADNSKNTAYLQMNSL |
| | | KPEDTAVYYCATGTFDVLRFLEWRLWGQGTLVTVSS |
| 323 | CXCR5-2-125 | EVQLVESGGGLVQPGGSLRLSCAASGFTFDYYAMGWFRQAPGK |
| | | ERELVAGISWNGGSIYYADSVKGRFTISADNSKNTAYLQMNSLK |
| | | PEDTAVYYCMRYYDSSGYSQDFDYWGQGTLVTVSS |
| 324 | CXCR5-2-126 | EVQLVESGGGLVQPGGSLRLSCAASGFTFEDYAMGWFRQAPGK |
| | | ERELVAAISGSGSITNYADSVKGRFTISADNSKNTAYLQMNSLKP |
| | | EDTAVYYCARGPEWTPPGDYFYYMDDWGQGTLVTVSS |
| 325 | CXCR5-2-127 | EVQLVESGGGLVQPGGSLRLSCAASGFTFEDYAMGWFRQAPGK |
| | | EREFVAAISSSGISTIYADSVKGRFTISADNSKNTAYLQMNSLKPE |
| | | DTAVYYCAREGCSSTSCYLDPWGQGTLVTVSS |
| 326 | CXCR5-2-128 | EVQLVESGGGLVQPGGSLRLSCAASGFTFEDYAMGWFRQAPGK |
| | | EREWVSGISSGGTTVYADSVKGRFTISADNSKNTAYLQMNSLKP |
| | | EDTAVYYCGRTSYYGDFEWGQGTLVTVSS |
| 327 | CXCR5-2-129 | EVQLVESGGGLVQPGGSLRLSCAASGFTFGHYAMGWFRQAPGK |
| | | EREFVAAINRSGDTTYYADSVKGRFTISADNSKNTAYLQMNSLK |
| | | PEDTAVYYCARGPEWTPPGDYFYYMDDWGQGTLVTVSS |
| 328 | CXCR5-2-130 | EVQLVESGGGLVQPGGSLRLSCAASGFTFRRYVMGWFRQAPGK |
| | | EREFVAAIRWSGGITWYADSVKGRFTISADNSKNTAYLQMNSLK |
| | | PEDTAVYYCARGPEWTPPGDYFYYMDDWGQGTLVTVSS |
| 329 | CXCR5-2-131 | EVQLVESGGGLVQPGGSLRLSCAASGFTFRRYVMGWFRQAPGK |
| | | EREFVAAIRWSGGITWYADSVKGRFTISADNSKNTAYLQMNSLK |
| | | PEDTAVYYCVRGRSRGTSGTTADWGQGTLVTVSS |
| 330 | CXCR5-2-132 | EVQLVESGGGLVQPGGSLRLSCAASGFTFRSYPMGWFRQAPGKE |
| | | REFVAAISGSDGSTYYADSVKGRFTISADNSKNTAYLQMNSLKP |
| | | EDTAVYYCARASSDYGDVSGPWGQGTLVTVSS |
| 331 | CXCR5-2-133 | EVQLVESGGGLVQPGGSLRLSCAASGFTFSDYPMGWFRQAPGKE |
| | | REAVAAIASMGGLTYYADSVKGRFTISADNSKNTAYLQMNSLKP |
| | | EDTAVYYCARLFAQYSDYDYVAEWGQGTLVTVSS |
| 332 | CXCR5-2-134 | EVQLVESGGGLVQPGGSLRLSCAASGFTFSDYPMGWFRQAPGKE |
| | | REAVAAIASMGGLTYYADSVKGRFTISADNSKNTAYLQMNSLKP |
| | | EDTAVYYCARSDYDVVSGLTNDYLYYLDDWGQGTLVTVSS |
| 333 | CXCR5-2-135 | EVQLVESGGGLVQPGGSLRLSCAASGFTFSEYGMGWFRQAPGK |
| | | EREFVAGVAWSSDFTAYADSVKGRFTISADNSKNTAYLQMNSL |
| | | KPEDTAVYYCARVGLGSCSTTSCFDYWGQGTLVTVSS |
| 334 | CXCR5-2-136 | EVQLVESGGGLVQPGGSLRLSCAASGFTFSGNWMGWFRQAPGK |
| | | EREGVSCIRWSGGQITYYADSVKGRFTISADNSKNTAYLQMNSL |
| | | KPEDTAVYYCTKGPTGPPRFFDFWGQGTLVTVSS |
| 335 | CXCR5-2-137 | EVQLVESGGGLVQPGGSLRLSCAASGFTFSNYWMGWFRQAPGK |
| | | ERELVATITSGGSTYYADSVKGRFTISADNSKNTAYLQMNSLKPE |
| | | DTAVYYCRAGASYWGQGTLVTVSS |
| 336 | CXCR5-2-138 | EVQLVESGGGLVQPGGSLRLSCAASGFTFSRYAMGWFRQAPGK |
| | | EREFVAAINYSGGSTNYADSVKGRFTISADNSKNTAYLQMNSLK |
| | | PEDTAVYYCAAVGAAGAVFWGQGTLVTVSS |
| 337 | CXCR5-2-139 | EVQLVESGGGLVQPGGSLRLSCAASGFTFSSDAMGWFRQAPGKE |
| | | RELVAAVSGTGTIAYYADSVKGRFTISADNSKNTAYLQMNSLKP |
| | | EDTAVYYCARGSGGGVDYWGQGTLVTVSS |
| 338 | CXCR5-2-140 | EVQLVESGGGLVQPGGSLRLSCAASGFTFSSDDYSMGWFRQAPG |
| | | KERELVAGVNWSGKDTYYADSVKGRFTISADNSKNTAYLQMNS |
| | | LKPEDTAVYYCARANKYYYDYYGVDVWGQGTLVTVSS |
| 339 | CXCR5-2-141 | EVQLVESGGGLVQPGGSLRLSCAASGYTYTTYSMGWFRQAPGQ |
| | | RTRICGGDYWSGKDTYYADSVKGRFTISADNSKNTAYLQMNSL |
| | | KPEDTAVYYCARGPDMIRSWYAWFDPWGQGTLVTVSS |
| 340 | CXCR5-2-142 | QVQLVESGGGLVQPGGSLRLSCAASGNIFINNAMGWFRQAPGKE |
| | | RELVAAINRSGGATSYADSVKGRFTISADNSKNTAYLQMNSLKP |
| | | EDTAVYYCARLFGSPSSSADYYYFDLWGQGTLVTVSS |
| 341 | CXCR5-2-143 | EVQLVESGGGLVQPGGSLRLSCAASGSIFSINAMGWFRQAPGKE |
| | | REFVAAISWSAGSTYYADSVKGRFTISADNSKNTAYLQMNSLKP |
| | | EDTAVYYCAKDRCGGDCNFSVLDWFDPWGQGTLVTVSS |
| 342 | CXCR5-2-144 | EVQLVESGGGLVQPGGSLRLSCAASGFNFDDYAMGWFRQAPGK |
| | | EREWVSEISSGGNKDYADSVKGRFTISADNSKNTAYLQMNSLKP |
| | | EDTAVYYCSAIISSTTGTDYFQNWGQGTLVTVSS |
| 343 | CXCR5-2-145 | EVQLVESGGGLVQPGGSLRLSCAASGRTFSNTLMGWFRQAPGK |
| | | EREAVAAISWSGDNTYYADSVKGRFTISADNSKNTAYLQMNSL |
| | | KPEDTAVYYCAKAGGYDYVWGSYPSDYWGQGTLVTVSS |
| 344 | CXCR5-2-146 | EVQLVESGGGLVQPGGSLRLSCAASGRTGTIYGMGWFRQAPGK |
| | | EREAVAAISWSDGSTYYADSVKGRFTISADNSKNTAYLQMNSLK |
| | | PEDTAVYYCARSDYDVVSGLTNDYLYYLDDWGQGTLVTVSS |
| 345 | CXCR5-2-147 | EVQLVESGGGLVQPGGSLRLSCAASGRTPSIIAMGWFRQAPGKE |
| | | RELVAGISSEGTTIYADSVKGRFTISADNSKNTAYLQMNSLKPED |
| | | TAVYYCVKVGEQTEYVDGTGYDYFYAMDVWGQGTLVTVSS |
| 346 | CXCR5-2-148 | EVQLVESGGGLVQPGGSLRLSCAASGSIDNIHAMGWFRQAPGKE |
| | | RELVAGITWSGDSTYYADSVKGRFTISADNSKNTAYLQMNSLKP |
| | | EDTAVYYCARSPGIRGPINHWGQGTLVTVSS |
| 347 | CXCR5-2-149 | EVQLVESGGGLVQPGGSLRLSCAASGSIDSIHVMGWFRQAPGKE |
| | | REFVAAISWTGGSTAYADSVKGRFTISADNSKNTAYLQMNSLKP |
| | | EDTAVYYCAKDFDYGDYWERDAFDIWGQGTLVTVSS |
| 348 | CXCR5-2-150 | EVQLVESGGGLVQPGGSLRLSCAASGSIDSIHVMGWFRQAPGKE |
| | | REFVAAISWTGGSTAYADSVKGRFTISADNSKNTAYLQMNSLKP |
| | | EDTAVYYCAKGRVGVYGDYLFDHWGQGTLVTVSS |
| 349 | CXCR5-17-3 | QVQLVQSGAEVKKPGSSVKVSCKASGGTFSSHVISWVRQAPGQ |
| | | GLEWMGEIIPLFGTTNYAQKFQGRVTITADESTSTAYMELSSLRS |
| | | EDTAVYYCARANQHFAKGLKGPTSSTVFQGTKGYHYYGMDVW |
| | | GQGTLVTVSS |
| 350 | CXCR5-17-11 | QVQLVQSGAEVKKPGSSVKVSCKASGGSFSSDAISWVRQAPGQG |
| | | LEWMGGIIPFFGTTNYAQKFQGRVTITADESTSTAYMELSSLRSE |
| | | DTAVYYCARDMYYDFSIAGDETFDVIGTRDEVVPADDAFDIWG |
| | | QGTLVTVSS |
| 351 | CXCR5-50-18 | EVQLVESGGGLVQAGGSLRLSCAASGRTFNNDHMGWFRQAPGK |
| | | EREFVAVIEIGGATNYADSVKGRFTISADNAKNTVYLQMNSLKP |
| | | EDTAVYYCASWDGRQVWGQGTQVTVSS |
| 352 | CXCR5-18-27 | EVQLVESGGGLVQPGGSLRLSCAASGLTFDDSAMGWFRQAPGK |
| | | EREFVAAMRWSGASTYYADSVKGRFTISADNSKNTAYLQMNSL |
| | | KPEDTAVYYCAAEDPSMGYYTLEEYEYDWGQGTLVTVSS |
| 353 | CXCR5-18-35 | EVQLVESGGGLVQPGGSLRLSCAASGRTLSKYRMGWFRQAPGK |
| | | EREFVAVIDTNGDNTLYADSVKGRFTISADNSKNTAYLQMNSLK |
| | | PEDTAVYYCAAALDGYSGSWGQGTLVTVSS |
| 354 | CXCR5-18-47 | EVQLVESGGGLVQPGGSLRLSCAASGLPFSRPVMGWFRQAPGKE |
| | | RELVAAIRGSGGSTEYADSVRGLFTITADNSKNTEHLKMNLLKPE |
| | | DTAVYYCASTRFAGRWYPDSKYRWGQGTLVTVST |
| 355 | CXCR5-18-48 | EVQLVESGGGLVQPGGSLRLSCAASGDISSIVAMGWFRQAPGKE |
| | | REFVAVVSGSGDDTYYADSVKGRFTISADNSKNTAYLQMNSLKP |
| | | EDTAVYYCATDEDYALGPNEFDWGQGTLVTVSS |
| 356 | CXCR5-16 | QVQLKESGPGLVAPSESLSITCTVSGFSLIDYGVNWIRQPPGKGLE |
| | | WLGVIWGDGTTYYNPSLKSRLSISKDNSKSQVFLKVTSLTTDDT |
| | | AMYYCARIVYWGQGTLVTVSA |

TABLE 10
CXCR5 Variably Light Chain Sequences

| SEQ | | |
|---|---|---|
| ID | | |
| NO | Variant | Sequence |
| 357 | CXCR5-1-1 | DIVMTQSPLSLPVSLGERASISCRSSQSLVHSDGNTYLEWYQQKP |
| | | GQSPKLLIYKVSNRASGVPDRFSGSGSGTDFTLKISRVEAEDVGV |
| | | YYCQQSSETPLTFGQGTKLEIK |
| 358 | CXCR5-1-2 | DIVMTQSPLSLPVSLGERASISCRSSQSLVHSNGNTYLAWYQQKP |
| | | GQSPKLLIYKASNRASGVPDRFSGSGSGTDFTLKISRVEAEDVGV |
| | | YYCQQSSSYPFTFGQGTKLEIK |
| 359 | CXCR5-1-3 | DIVMTQSPLSLPVSLGERASISCRSSQSLVHSDGNTYLAWYQQKP |
| | | GQSPKLLIYKASNRFSGVPDRFSGSGSGTDFTLKISRVEAEDVGV |
| | | YYCQQGSETPLTFGQGTKLEIK |
| 360 | CXCR5-1-4 | DIVMTQSPLSLPVSLGERASISCRSSQSLVHSNGNTYLAWYQQKP |
| | | GQSPKLLIYKVSNRASGVPDRFSGSGSGTDFTLKISRVEAEDVGV |
| | | YYCQQGYSTPLTFGQGTKLEIK |
| 361 | CXCR5-1-5 | DIVMTQSPLSLPVSLGERASISCRSSQSLVHSDGKTYLEWYQQKP |
| | | GQSPKLLIYKASNRASGVPDRFSGSGSGTDFTLKISRVEAEDVGV |
| | | YYCFQSSSTPFTFGQGTKLEIK |
| 362 | CXCR5-1-6 | DIVMTQSPLSLPVSLGERASISCRSSQSLVNSDGKTYLHWYQQKP |
| | | GQSPKLLIYKASNRASGVPDRFSGSGSGTDFTLKISRVEAEDVGV |
| | | YYCFQGSEYPFTFGQGTKLEIK |
| 363 | CXCR5-1-7 | DIVMTQSPLSLPVSLGERASISCRSSQSLVNSDGNTYLEWYQQKP |
| | | GQSPKLLIYKASNRASGVPDRFSGSGSGTDFTLKISRVEAEDVGV |
| | | YYCFQGSEYPFTFGQGTKLEIK |
| 364 | CXCR5-1-8 | DIVMTQSPLSLPVSLGERASISCRSSQSLVHSNGNTYLHWYQQKP |
| | | GQSPKLLIYKVSNRASGVPDRFSGSGSGTDFTLKISRVEAEDVGV |
| | | YYCFQSYHYPLTFGQGTKLEIK |
| 365 | CXCR5-1-9 | DIVMTQSPLSLPVSLGERASISCRSSQSLVNSDGNTYLHWYQQKP |
| | | GQSPKLLIYKASNRASGVPDRFSGSGSGTDFTLKISRVEAEDVGV |
| | | YYCQQGSSTPLTFGQGTKLEIK |
| 366 | CXCR5-1-10 | DIVMTQSPLSLPVSLGERASISCRSSQSLVNSNGKTYLAWYQQKP |
| | | GQSPKLLIYKASNRFSGVPDRFSGSGSGTDFTLKISRVEAEDVGV |
| | | YYCQQSSHVPFTFGQGTKLEIK |
| 367 | CXCR5-1-11 | DIVMTQSPLSLPVSLGERASISCRSSQSLVNSNGKTYLAWYQQKP |
| | | GQSPKLLIYKASNRFSGVPDRFSGSGSGTDFTLKISRVEAEDVGV |
| | | YYCQQGYHTPLTFGQGTKLEIK |
| 368 | CXCR5-1-12 | DIVMTQSPLSLPVSLGERASISCRSSQSLVNSDGKTYLHWYQQKP |
| | | GQSPKLLIYKVSNRASGVPDRFSGSGSGTDFTLKISRVEAEDVGV |
| | | YYCQQGYETPFTFGQGTKLEIK |
| 369 | CXCR5-1-13 | DIVMTQSPLSLPVSLGERASISCRSSQSLVNSDGKTYLEWYQQKP |
| | | GQSPKLLIYKASNRFSGVPDRFSGSGSGTDFTLKISRVEAEDVGV |
| | | YYCFQSSSVPLTFGQGTKLEIK |
| 370 | CXCR5-1-14 | DIVMTQSPLSLPVSLGERASISCRSSQSLVNSNGKTYLAWYQQKP |
| | | GQSPKLLIYKVSNRASGVPDRFSGSGSGTDFTLKISRVEAEDVGV |
| | | YYCQQSSEYPFTFGQGTKLEIK |
| 371 | CXCR5-1-15 | DIVMTQSPLSLPVSLGERASISCRSSQSLVHSNGKTYLHWYQQKP |
| | | GQSPKLLIYKASNRASGVPDRFSGSGSGTDFTLKISRVEAEDVGV |
| | | YYCFQGYHTPLTFGQGTKLEIK |
| 372 | CXCR5-1-16 | DIVMTQSPLSLPVSLGERASISCRSSQSLVNSNGNTYLHWYQQKP |
| | | GQSPKLLIYKVSNRASGVPDRFSGSGSGTDFTLKISRVEAEDVGV |
| | | YYCFQGSHYPFTFGQGTKLEIK |
| 373 | CXCR5-1-17 | DIVMTQSPLSLPVSLGERASISCRSSQSLVHSDGNTYLHWYQQKP |
| | | GQSPKLLIYKVSNRASGVPDRFSGSGSGTDFTLKISRVEAEDVGV |
| | | YYCQQGYETPFTFGQGTKLEIK |
| 374 | CXCR5-1-18 | DIVMTQSPLSLPVSLGERASISCRSSQSLVHSNGKTYLAWYQQKP |
| | | GQSPKLLIYKASNRASGVPDRFSGSGSGTDFTLKISRVEAEDVGV |
| | | YYCQQSYHVPFTFGQGTKLEIK |
| 375 | CXCR5-1-19 | DIVMTQSPLSLPVSLGERASISCRSSQSLVNSDGNTYLHWYQQKP |
| | | GQSPKLLIYKASNRASGVPDRFSGSGSGTDFTLKISRVEAEDVGV |
| | | YYCQQSSHTPFTFGQGTKLEIK |
| 376 | CXCR5-1-20 | DIVMTQSPLSLPVSLGERASISCRSSQSLVNSDGKTYLEWYQQKP |
| | | GQSPKLLIYKASNRASGVPDRFSGSGSGTDFTLKISRVEAEDVGV |
| | | YYCFQGSHYPFTFGQGTKLEIK |
| 377 | CXCR5-1-21 | DIVMTQSPLSLPVSLGERASISCRSSQSLVNSDGNTYLHWYQQKP |
| | | GQSPKLLIYKVSNRFSGVPDRFSGSGSGTDFTLKISRVEAEDVGV |
| | | YYCFQGYETPLTFGQGTKLEIK |
| 378 | CXCR5-1-22 | DIVMTQSPLSLPVSLGERASISCRSSQSLVNSDGNTYLHWYQQKP |
| | | GQSPKLLIYKASNRASGVPDRFSGSGSGTDFTLKISRVEAEDVGV |
| | | YYCQQSSHVPFTFGQGTKLEIK |
| 379 | CXCR5-1-23 | DIVMTQSPLSLPVSLGERASISCRSSQSLVNSDGNTYLEWYQQKP |
| | | GQSPKLLIYKVSNRFSGVPDRFSGSGSGTDFTLKISRVEAEDVGV |
| | | YYCFQGYHTPFTFGQGTKLEIK |
| 380 | CXCR5-1-24 | DIVMTQSPLSLPVSLGERASISCRSSQSLVNSNGKTYLAWYQQKP |
| | | GQSPKLLIYKVSNRASGVPDRFSGSGSGTDFTLKISRVEAEDVGV |
| | | YYCQQSYHTPFTFGQGTKLEIK |
| 381 | CXCR5-1-25 | DIVMTQSPLSLPVSLGERASISCRSSQSLVNSDGNTYLAWYQQKP |
| | | GQSPKLLIYKASNRASGVPDRFSGSGSGTDFTLKISRVEAEDVGV |
| | | YYCFQGSSTPLTFGQGTKLEIK |
| 382 | CXCR5-1-26 | DIVMTQSPLSLPVSLGERASISCRSSQSLVNSNGNTYLHWYQQKP |
| | | GQSPKLLIYKVSNRFSGVPDRFSGSGSGTDFTLKISRVEAEDVGV |
| | | YYCFQSYSTPFTFGQGTKLEIK |
| 383 | CXCR5-1-27 | YGSMTQSPLSLPVSLGERASISCRSSQSLVHSNGNTYLHWYQQKP |
| | | GQSPKLLIYKASNRFSGVPDRFSGSGSGTDFTLKISRVEAEDVGV |
| | | YYCQQSYSTPLTFGQGTKLEIK |
| 384 | CXCR5-1-28 | DIVMTQSPLSLPVSLGERASISCRSSQSLVNSNGKTYLAWYQQKP |
| | | GQSPKLLIYKASNRASGVPDRFSGSGSGTDFTLKISRVEAEDVGV |
| | | YYCQQSYEVPLTFGQGTKLEIK |
| 385 | CXCR5-1-29 | HIVMTQSPLSLPVSLGERASISCRSSQSLVHSNGKTYLHWYQQKP |
| | | GQSPKLLIYKASNRASGVPDRFSGSGSGTDFTLKISRVEAEDVGV |
| | | YYCQQSSSYPFTFGQGTKLEIK |
| 386 | CXCR5-1-30 | DIVMTQSPLSLPVSLGERASISCRSSQSLVHSDGKTYLEWYQQKP |
| | | GQSPKLLIYKVSNRASGVPDRFSGSGSGTDFTLKISRVEAEDVGV |
| | | YYCFQGSHYPFTFGQGTKLEIK |
| 387 | CXCR5-1-31 | DIVMTQSPLSLPVSLGERASISCRSSQSLVNSDGKTYLHWYQQKP |
| | | GQSPKLLIYKVSNRASGVPDRFSGSGSGTDFTLKISRVEAEDVGV |
| | | YYCQQGYSTPFTFGQGTKLEIK |
| 388 | CXCR5-1-32 | DIVMTQSPLSLPVSLGERASISCRSSQSLVHSDGKTYLAWYQQKP |
| | | GQSPKLLIYKASNRFSGVPDRFSGSGSGTDFTLKISRVEAEDVGV |
| | | YYCQQSYHVPFTFGQGTKLEIK |
| 389 | CXCR5-1-33 | DIVMTQSPLSLPVSLGERASISCRSSQSLVHSDGKTYLEWYQQKP |
| | | GQSPKLLIYKASNRFSGVPDRFSGSGSGTDFTLKISRVEAEDVGV |
| | | YYCQQSSHYPLTFGQGTKLEIK |
| 390 | CXCR5-1-34 | DIVMTQSPLSLPVSLGERASISCRSSQSLVNSNGKTYLAWYQQKP |
| | | GQSPKLLIYKVSNRASGVPDRFSGSGSGTDFTLKISRVEAEDVGV |
| | | YYCQQGSHVPFTFGQGTKLEIK |
| 391 | CXCR5-1-35 | DIVMTQSPLSLPVSLGERASISCRSSQSLVNSDGNTYLHWYQQKP |
| | | GQSPKLLIYKASNRASGVPDRFSGSGSGTDFTLKISRVEAEDVGV |
| | | YYCQQSSSTPFTFGQGTKLEIK |
| 392 | CXCR5-1-36 | DIVMTQSPLSLPVSLGERASISCRSSQSLVNSNGNTYLHWYQQKP |
| | | GQSPKLLIYKASNRASGVPDRFSGSGSGTDFTLKISRVEAEDVGV |
| | | YYCQQGYHYPLTFGQGTKLEIK |
| 393 | CXCR5-1-37 | DIVMTQSPLSLPVSLGERASISCRSSQSLVNSNGKTYLAWYQQKP |
| | | GQSPKLLIYKASNRASGVPDRFSGSGSGTDFTLKISRVEAEDVGV |
| | | YYCQQGSEYPLTFGQGTKLEIK |
| 394 | CXCR5-1-38 | DIVMTQSPLSLPVSLGERASISCRSSQSLVHSNGNTYLEWYQQKP |
| | | GQSPKLLIYKVSNRASGVPDRFSGSGSGTDFTLKISRVEAEDVGV |
| | | YYCQQGSSVPFTFGQGTKLEIK |
| 395 | CXCR5-1-39 | DIVMTQSPLSLPVSLGERASISCRSSQSLVNSNGNTYLAWYQQKP |
| | | GQSPKLLIYKASNRASGVPDRFSGSGSGTDFTLKISRVEAEDVGV |
| | | YYCFQGYEVPFTFGQGTKLEIK |
| 396 | CXCR5-1-40 | DIVMTQSPLSLPVSLGERASISCRSSQSLVNSNGNTYLEWYQQKP |
| | | GQSPKLLIYKVSNRFSGVPDRFSGSGSGTDFTLKISRVEAEDVGV |
| | | YYCQQSSEYPLTFGQGTKLEIK |
| 397 | CXCR5-1-41 | DIVMTQSPLSLPVSLGERASISCRSSQSLVHSNGKTYLEWYQQKP |
| | | GQSPKLLIYKASNRASGVPDRFSGSGSGTDFTLKISRVEAEDVGV |
| | | YYCFQGSHYPLTFGQGTKLEIK |
| 398 | CXCR5-1-42 | YGSMTQSPLSLPVSLGERASISCRSSQSLVHSNGNTYLHWYQQKP |
| | | GQSPKLLIYKVSNRFSGVPDRFSGSGSGTDFTLKISRVEAEDVGV |
| | | YYCQQSSSYPFTFGQGTKLEIK |
| 399 | CXCR5-1-43 | DIVMTQSPLSLPVSLGERASISCRSSQSLVHSNGNTYLHWYQQKP |
| | | GQSPKLLIYKASNRASGVPDRFSGSGSGTDFTLKISRVEAEDVGV |
| | | YYCFQSYEYPFTFGQGTKLEIK |
| 400 | CXCR5-1-44 | DIVMTQSPLSLPVSLGERASISCRSSQSLVHSNGKTYLHWYQQKP |
| | | GQSPKLLIYKASNRASGVPDRFSGSGSGTDFTLKISRVEAEDVGV |
| | | YYCFQSSSTPFTFGQGTKLEIK |
| 401 | CXCR5-1-45 | DIVMTQSPLSLPVSLGERASISCRSSQSLVHSDGKTYLHWYQQKP |
| | | GQSPKLLIYKASNRFSGVPDRFSGSGSGTDFTLKISRVEAEDVGV |
| | | YYCQQSYHYPFTFGQGTKLEIK |
| 402 | CXCR5-1-46 | DIVMTQSPLSLPVSLGERASISCRSSQSLVHSDGNTYLAWYQQKP |
| | | GQSPKLLIYKVSNRASGVPDRFSGSGSGTDFTLKISRVEAEDVGV |
| | | YYCFQGSHYPFTFGQGTKLEIK |
| 403 | CXCR5-1-47 | DIVMTQSPLSLPVSLGERASISCRSSQSLVHSNGNTYLAWYQQKP |
| | | GQSPKLLIYKASNRFSGVPDRFSGSGSGTDFTLKISRVEAEDVGV |
| | | YYCQQSYSYPLTFGQGTKLEIK |
| 404 | CXCR5-1-48 | DIVMTQSPLSLPVSLGERASISCRSSQSLVNSNGNTYLAWYQQKP |
| | | GQSPKLLIYKVSNRASGVPDRFSGSGSGTDFTLKISRVEAEDVGV |
| | | YYCFQSYHTPLTFGQGTKLEIK |
| 405 | CXCR5-1-49 | DIVMTQSPLSLPVSLGERASISCRSSQSLVNSDGNTYLAWYQQKP |
| | | GQSPKLLIYKASNRFSGVPDRFSGSGSGTDFTLKISRVEAEDVGV |
| | | YYCFQSYEVPLTFGQGTKLEIK |
| 406 | CXCR5-1-50 | DIVMTQSPLSLPVSLGERASISCRSSQSLVHSDCKTYLAWYQQKP |
| | | GQSPKLLIYKASNRFSGVPDRFSGSGSGTDFTLKISRVEAEDVGV |
| | | YYCQQGSEYPFTFGQGTKLEIK |
| 407 | CXCR5-1-51 | DIVMTQSPLSLPVSLGERASISCRSSQSLVHSDGNTYLEWYQQKP |
| | | GQSPKLLIYKASNRASGVPDRFSGSGSGTDFTLKISRVEAEDVGV |
| | | YYCQQSSSVPLTFGQGTKLEIK |
| 408 | CXCR5-1-52 | DIVMTQSPLSLPVSLGERASISCRSSQSLVNSDGKTYLAWYQQKP |
| | | GQSPKLLIYKVSNRFSGVPDRFSGSGSGTDFTLKISRVEAEDVGV |
| | | YYCQQGSHVPFTFGQGTKLEIK |
| 409 | CXCR5-1-53 | DIVMTQSPLSLPVSLGERASISCRSSQSLVNSDGKTYLEWYQQKP |
| | | GQSPKLLIYKVSNRASGVPDRFSGSGSGTDFTLKISRVEAEDVGV |
| | | YYCQQSYSVPLTFGQGTKLEIK |
| 410 | CXCR5-1-54 | DIVMTQSPLSLPVSLGERASISCRSSQSLVNSNGKTYLAWYQQKP |
| | | GQSPKLLIYKVSNRFSGVPDRFSGSGSGTDFTLKISRVEAEDVGV |
| | | YYCQQSYSVPLTFGQGTKLEIK |
| 411 | CXCR5-1-55 | DIVMTQSPLSLPVSLGERASISCRSSQSLVNSNGNTYLAWYQQKP |
| | | GQSPKLLIYKVSNRASGVPDRFSGSGSGTDFTLKISRVEAEDVGV |
| | | YYCFQSSHTPFTFGQGTKLEIK |
| 412 | CXCR5-1-56 | DIVMTQSPLSLPVSLGERASISCRSSQSLVNSDGNTYLEWYQQKP |
| | | GQSPKLLIYKASNRASGVPDRFSGSGSGTDFTLKISRVEAEDVGV |
| | | YYCFQGSSTPLTFGQGTKLEIK |
| 413 | CXCR5-1-57 | DIVMTQSPLSLPVSLGERASISCRSSQSLVHSNGKTYLAWYQQKP |
| | | GQSPKLLIYKASNRFSGVPDRFSGSGSGTDFTLKISRVEAEDVGV |
| | | YYCQQSSSYPFTFGQGTKLEIK |
| 414 | CXCR5-1-58 | DIVMTQSPLSLPVSLGERASISCRSSQSLVHSDGNTYLAWYQQKP |
| | | GQSPKLLIYKASNRFSGVPDRFSGSGSGTDFTLKISRVEAEDVGV |
| | | YYCQQSYEYPFTFGQGTKLEIK |
| 415 | CXCR5-1-59 | DIVMTQSPLSLPVSLGERASISCRSSQSLVNSDGKTYLAWYQQKP |
| | | GQSPKLLIYKASNRASGVPDRFSGSGSGTDFTLKISRVEAEDVGV |
| | | YYCFQSSHTPLTFGQGTKLEIK |
| 416 | CXCR5-1-60 | DIVMTQSPLSLPVSLGERASISCRSSQSLVNSDGKTYLHWYQQKP |
| | | GQSPKLLIYKVSNRFSGVPDRFSGSGSGTDFTLKISRVEAEDVGV |
| | | YYCQQSSETPFTFGQGTKLEIK |
| 417 | CXCR5-1-61 | DIVMTQSPLSLPVSLGERASISCRSSQSLVNSNGNTYLAWYQQKP |
| | | GQSPKLLIYKASNRASGVPDRFSGSGSGTDFTLKISRVEAEDVGV |
| | | YYCFQSYSVPLTFGQGTKLEIK |
| 418 | CXCR5-1-62 | DIVMTQSPLSLPVSLGERASISCRSSQSLVNSDGKTYLEWYQQKP |
| | | GQSPKLLIYKASNRFSGVPDRFSGSGSGTDFTLKISRVEAEDVGV |
| | | YYCFQSYETPLTFGQGTKLEIK |
| 419 | CXCR5-1-63 | DIVMTQSPLSLPVSLGERASISCRSSQSLVHSNGNTYLAWYQQKP |
| | | GQSPKLLIYKASNRFSGVPDRFSGSGSGTDFTLKISRVEAEDVGV |
| | | YYCFQSSSTPFTFGQGTKLEIK |
| 420 | CXCR5-1-64 | DIVMTQSPLSLPVSLGERASISCRSSQSLVHSNGNTYLHWYQQKP |
| | | GQSPKLLIYKASNRASGVPDRFSGSGSGTDFTLKISRVEAEDVGV |
| | | YYCQQSYHYPFTFGQGTKLEIK |
| 421 | CXCR5-1-65 | DIVMTQSPLSLPVSLGERASISCRSSQSLVHSDGNTYLAWYQQKP |
| | | GQSPKLLIYKASNRFSGVPDRFSGSGSGTDFTLKISRVEAEDVGV |
| | | YYCQQESSTPFTFGQGTKLEIK |
| 422 | CXCR5-1-66 | DIVMTQSPLSLPVSLGERASISCRSSQSLVHSDGKTYLEWYQQKP |
| | | GQSPKLLIYKASNRFSGVPDRFSGSGSGTDFTLKISRVEAEDVGV |
| | | YYCQQSYHYPLTFGQGTKLEIK |
| 423 | CXCR5-1-67 | DIVMTQSPLSLPVSLGERASISCRSSQSLVNSDGKTYLHWYQQKP |
| | | GQSPKLLIYKASNRASGVPDRFSGSGSGTDFTLKISRVEAEDVGV |
| | | YYCQQSYHTPLTFGQGTKLEIK |
| 424 | CXCR5-1-68 | DIVMTQSPLSLPVSLGERASISCRSSQSLVHSDGNTYLAWYQQKP |
| | | GQSPKLLIYKVSNRASGVPDRFSGSGSGTDFTLKISRVEAEDVGV |
| | | YYCQQSYEYPFTFGQGTKLEIK |
| 425 | CXCR5-1-69 | DIVMTQSPLSLPVSLGERASISCRSSQSLVHSDGKTYLHWYQQKP |
| | | GQSPKLLIYKVSNRFSGVPDRFSGSGSGTDFTLKISRVEAEDVGV |
| | | YYCQQGSSTPLTFGQGTKLEIK |
| 426 | CXCR5-1-70 | DIVMTQSPLSLPVSLGERASISCRSSQSLVHSNGKTYLHWYQQKP |
| | | GQSPKLLIYKVSNRFSGVPDRFSGSGSGTDFTLKISRVEAEDVGV |
| | | YYCQQGSSIPLTFGQGTKLEIK |
| 427 | CXCR5-1-71 | DIVMTQSPLSLPVSLGERASISCRSSQSLVHSDGKTYLAWYQQKP |
| | | GQSPKLLIYKASNRASGVTDRFSGSGSGTDFTLKISRVEAEDVGV |
| | | YYCFQSSSTPLTFGQGTKLEIK |
| 428 | CXCR5-1-72 | DIVMTQSPLSLPVSLGERASISCRSSQSLVHSDGKTYLHWYQQKP |
| | | GQSPKLLIYKVSNRASGVPDRFSGSGSGTDFTLKISRVEAEDVGV |
| | | YYCQQSYHYPLTFGQGTKLEIK |
| 429 | CXCR5-1-73 | DIVMTQSPLSLPVSLGERASISCRSSQSLVNSDGNTYLAWYQQKP |
| | | GQSPKLLIYKASNRFSGVPDRFSGSGSGTDFTLKISRVEAEDVGV |
| | | YYCFQGYETPLTFGQGTKLEIK |
| 430 | CXCR5-1-74 | DIVMTQSPLSLPVSLGERASISCRSSQSLVHSNGNTYLAWYQQKP |
| | | GQSPKLLIYKVSNRASGVPDRFSGSGSGTDFTLKISRVEAEDVGV |
| | | YYCQQGYHTPLTFGQGTKLEIK |
| 431 | CXCR5-1-75 | DIVMTQSPLSLPVSLGERASISCRSSQSLVHSDGNTYLHWYQQKP |
| | | GQSPKLLIYKASNRASGVPDRFSGSGSGTDFTLKISRVEAEDVGV |
| | | YYCQQSSHVPFTFGQGTKLEIK |
| 432 | CXCR5-1-76 | DIVMTQSPLSLPVSLGERASISCRSSQSLVNSDGKTYLAWYQQKP |
| | | GQSPKLLIYKASNRFSGVPDRFSGSGSGTDFTLKISRVEAEDVGV |
| | | YYCQQGYSYPLTFGQGTKLEIK |
| 433 | CXCR5-1-77 | DIVMTQSPLSLPVSLGERASISCRSSQSLVNSNGNTYLEWYQQKP |
| | | GQSPKLLIYKVSNRFSGVPDRFSGSGSGTDFTLKISRVEAEDVGV |
| | | YYCQQGSHVPFTFGQGTKLEIK |
| 434 | CXCR5-1-78 | DIVMTQSPLSLPVSLGERASISCRSSQSLVNSNGKTYLAWYQQKP |
| | | GQSPKLLIYKVSNRFSGVPDRFSGSGSGTDFTLKISRVEAEDVGV |
| | | YYCQQGYSVPLTFGQGTKLEIK |
| 435 | CXCR5-1-79 | DIVMTQSPLSLPVSLGERASISCRSSQSLVHSNGKTYLEWYQQKP |
| | | GQSPKLLIYKVSNRASGVPDRFSGSGSGTDFTLKISRVEAEDVGV |
| | | YYCFQSYSVPFTFGQGTKLEIK |
| 436 | CXCR5-1-80 | DIVMTQSPLSLPVSLGERASISCRSSQSLVHSDGNTYLAWYQQKP |
| | | GQSPKLLIYKVSNRASGVPDRFSGSGSGTDFTLKISRVEAEDVGV |
| | | YYCFQSYSYPLTFGQGTKLEIK |
| 437 | CXCR5-1-81 | DIVMTQSPLSLPVSLGERASISCRSSQSLVNSNGKTYLAWYQQKP |
| | | GQSPKLLIYKASNRFSGVPDRFSGSGSGTDFTLKISRVEAEDVGV |
| | | YYCQQSYHYPLTFGQGTKLEIK |
| 438 | CXCR5-1-82 | DIVMTQSPLSLPVSLGERASISCRSSQSLVNSNSNTYLEWYQQKP |
| | | GQSPKLLIYKASNRFSGVPDRFSGSGSGTDFTLKISRVEAEDVGV |
| | | YYCQQSSHVPFTFGQGTKLEIK |
| 439 | CXCR5-1-83 | DIVMTQSPLSLPVSLGERASISCRSSQSLVNSNGNTYLHWYQQKP |
| | | GQSPKLLIYKVSNRASGVPDRFSGSGSGTDFTLKISRVEAEDVGV |
| | | YYCQQGSHVPFTFGQGTKLEIK |
| 440 | CXCR5-1-84 | DIVMTQSPLSLPVSLGERASISCRSSQSLVHSNGKTYLHWYQQKP |
| | | GQSPKLLIYKASNRASGVPDRFSGSGSGTDFTLKISRVEAEDVGV |
| | | YYCQQGYHVPFTFGQGTKLEIK |
| 441 | CXCR5-1-85 | DIVMTQSPLSLPVSLGERASISCRSSQSLVNSDGNTYLEWYQQKP |
| | | GQSPKLLIYKASNRASGVPDRFSGSGSGTDFTLKISRVEAEDVGV |
| | | YYCQQSYHVPFTFGQGTKLEIK |
| 442 | CXCR5-1-86 | DIVMTQSPLSLPVSLGERASISCRSSQSLVNSDGNTYLHWYQQKP |
| | | GQSPKLLIYKVSNRASGVPDRFSGSGSGTDFTLKISRVEAEDVGV |
| | | YYCQQGYEYPFTFGQGTKLEIK |
| 443 | CXCR5-1-87 | DIVMTQSPLSLPVSLGERASISCRSSQSLVHSNGKTYLAWYQQKP |
| | | GQSPKLLIYKASNRFSGVPDRFSGSGSGTDFTLKISRVEAEDVGV |
| | | YYCQQSYSYALTFGQGTKLEIK |
| 444 | CXCR5-1-88 | DIVMTQSPLSLPVSLGERASISCRSSQSLVNSDGKTYLHWYQQKP |
| | | GQSPKLLIYKASNRASGVPDRFSGSGSGTDFTLKISRVEAEDVGV |
| | | YYCQQGSEVPFTFGQGTKLEIK |
| 445 | CXCR5-1-89 | DIVMTQSPLSLPVSLGERASISCRSSQSLVHSDGKTYLAWYQQKP |
| | | GQSPKLLIYKVSNRASGVPDRFSGSGSGTDFTLKISRVEAEDVGV |
| | | YYCQQSYSVPFTFGQGTKLEIK |
| 446 | CXCR5-1-90 | DIVMTQSPLSLPVSLGERASISCRSSQSLVHSDGKTYLHWYQQKP |
| | | GQSPKLLIYKVSNRFSGVPDRFSGSGSGTDFTLKISRVEAEDVGV |
| | | YYCFQGYSYPFTFGQGTKLEIK |
| 447 | CXCR5-1-91 | DIVMTQSPLSLPVSLGERASISCRSSQSLVHSDGNTYLAWYQQKP |
| | | GQSPKLLIYKVSNRASGVPDRFSGSGSGTDFTLKISRVEAEDVGV |
| | | YYCQQGSSYPLTFGQGTKLEIK |
| 448 | CXCR5-1-92 | DIVMTQSPLSLPVSLGERASISCRSSQSLVNSDGNTYLHWYQQKP |
| | | GQSPKLLIYKVSNRFSGVPDRFSGSGSGTDFTLKISRVEAEDVGV |
| | | YYCQQSYHTPFTFGQGTKLEIK |
| 449 | CXCR5-1-93 | DIVMTQSPLSLPVSLGERASISCRSSQSLVNSNGNTYLHWYQQKP |
| | | GQSPKLLIYKASNRASGVPDRFSGSGSGTDFTLKISRVEAEDVGV |
| | | YYCQQGYEYPLTFGQGTKLEIK |
| 450 | CXCR5-1-94 | DIVMTQSPLSLPVSLGERASISCRSSQSLVHSDGNTYLAWYQQKP |
| | | GQSPKLLIYKVSNRASGVPDRFSGSGSGTDFTLKISRVEAEDVGV |
| | | YYCQQSYSYPLTFGQGTKLEIK |
| 451 | CXCR5-1-95 | DIVMTQSPLSLPVSLGERASISCRSSQSLVHSNGNTYLHWYQQKP |
| | | GQSPKLLIYKASNRASGVPDRFSGSGSGTDFTLKISRVEAEDVGV |
| | | YYCQQGSEYPFTFGQGTKLEIK |
| 452 | CXCR5-1-96 | DIVMTQSPLSLPVSLGERASISCRSSQSLVHSNGKTYLEWYQQKP |
| | | GQSPKLLIYKASNRASGVPDRFSGSGSGTDFTLKISRVEAEDVGV |
| | | YYCFQSSSTPFTFGQGTKLEIK |
| 453 | CXCR5-1-97 | DIVMTQSPLSLPVSLGERASISCRSSQSLVHSDGKTYLAWYQQKP |
| | | GQSPKLLIYKVSNRFSGVPDRFSGSGSGTDFTLKISRVEAEDVGV |
| | | YYCQQSYSYPLTFGQGTKLEIK |
| 454 | CXCR5-1-98 | DIVMTQSPLSLPVSLGERASISCRSSQSLVHSDGNTYLEWYQQKP |
| | | GQSPKLLIYKVSNRASGVPDRFSGSGSGTDFTLKISRVEAEDVGV |
| | | YYCQQGYSVPFTFGQGTKLEIK |
| 455 | CXCR5-1-99 | DIVMTQSPLSLPVSLGERASISCRSSQSLVNSDGKTYLEWYQQKP |
| | | GQSPKLLIYKASNRFSGVPDRFSGSGSGTDFTLKISRVEAEDVGV |
| | | YYCQQSYSVPFTFGQGTKLEIK |
| 456 | CXCR5-1-100 | DIVMTQSPLSLPVSLGERASISCRSSQSLVHSNGNTYLHWYQQKP |
| | | GQSPKLLIYKVSNRASGVPDRFSGSGSGTDFTLKISRVEAEDVGV |
| | | YYCFQSSSYPFTFGQGTKLEIK |
| 457 | CXCR5-1-101 | DIVMTQSPLSLPVSLGERASISCRSSQSLVNSNGNTYLHWYQQKP |
| | | GQSPKLLIYKASNRASGVPDRFSGSGSGTDFTLKISRVEAEDVGV |
| | | YYCQQGYHVPFTFGQGTKLEIK |
| 458 | CXCR5-1-102 | DIVMTQSPLSLPVSLGERASISCRSSQSLVHSDGNTYLEWYQQKP |
| | | GQSPKLLIYKVSNRFSGVPDRFSGSGSGTDFTLKISRVEAEDVGV |
| | | YYCQQSYSTPFTFGQGTKLEIK |
| 459 | CXCR5-1-103 | DIVMTQSPLSLPVSLGERASISCRSSQSLVNSNGKTYLEWYQQKP |
| | | GQSPKLLIYKVSNRASGVPDRFSGSGSGTDFTLKISRVEAEDVGV |
| | | YYCQQSYHYPFTFGQGTKLEIK |
| 460 | CXCR5-1-104 | DIVMTQSPLSLPVSLGERASISCRSSQSLVHSDGNTYLHWYQQKP |
| | | GQSPKLLIYKVSNRFSGVPDRFSGSGSGTDFTLKISRVEAEDVGV |
| | | YYCQQSSHYPLTFGQGTKLEIK |
| 461 | CXCR5-1-105 | DIVMTQSPLSLPVSLGERASISCRSSQSLVHSDGKTYLAWYQQKP |
| | | GQSPKLLIYKASNRFSGVPDRFSGSGSGTDFTLKISRVEAEDVGV |
| | | YYYFQGSHTPFTFGQGTKLEIK |
| 462 | CXCR5-1-106 | DIVMTQSPLSLPVSLGERASISCRSSQSLVHSDGKTYLHWYQQKP |
| | | GQSPKLLIYKASNRFSGVPDRFSGSGSGTDFTLKISRVEAEDVGV |
| | | YYCFQGSSVPLTFGQGTKLEIK |
| 463 | CXCR5-1-107 | DIVMTQSPLSLPVSLGERASISCRSSQSLVNSNGNTYLEWYQQKP |
| | | GQSPKLLIYKASNRFSGVPDRFSGSGSGTDFTLKISRVEAEDVGV |
| | | YYCFQSYHVPFTFGQGTKLEIK |
| 464 | CXCR5-1-108 | DIVMTQSPLSLPVSLGERASISCRSSQSLVHSNGKTYLAWYQQKP |
| | | GQSPKLLIYKASNRLSGVPDRFSGSGSGTDFTLKISRVEAEDVGV |
| | | YYCQQSYSTPFTFGQGTKLEIK |
| 465 | CXCR5-1-109 | DIVMTQSPLSLPVSLGERASISCRSSQSLVNSNGNTYLHWYQQKP |
| | | GQSPKLLIYKASNRASGVPDRFSGSGSGTDFTLKISRVEAEDVGV |
| | | YYCQQGSEYPFTFGQGTKLEIK |
| 466 | CXCR5-1-110 | DIVMTQSPLSLPVSLGERASISCRSSQSLVNSDGKTYLHWYQQKP |
| | | GQSPKLLIYKVSNRFSGVPDRFSGSGSGTDFTLKISRVEAEDVGV |
| | | YYCQQSSHVPFTFGQGTKLEIK |
| 467 | CXCR5-1-111 | DIVMTQSPLSLPVSLGERASISCRSSQSLVNSDGNTYLAWYQQKP |
| | | GQSPKLLIYKASNRASGVPDRFSGSGSGTDFTLKISRVEAEDVGV |
| | | YYCQQGYSTPFTFGQGTKLEIK |
| 468 | CXCR5-1-112 | DIVMTQSPLSLPVSLGERASISCRSSQSLVNSDGNTYLHWYQQKP |
| | | GQSPKLLIYKASNRASGVPDRFSGSGSGTDFTLKISRVEAEDVGV |
| | | YYCQQGSEVPLTFGQGTKLEIK |
| 469 | CXCR5-1-113 | DIVMTQSPLSLPVSLGERASISCRSSQSLVHSNGKTYLAWYQQKP |
| | | GQSPKLLIYKASNRFSGVPDRFSGSGSGTDFTLKISRVEAEDVGV |
| | | YYCFQGYSTPLTFGQGTKLEIK |
| 470 | CXCR5-1-114 | DIVMTQSPLSLPVSLGERASISCRSSQSLVHSNGKTYLHWYQQKP |
| | | GQSPKLLIYKVSNRASGVPDRFSGSGSGTDFTLKISRVEAEDVGV |
| | | YYCFQSSEVPFTFGQGTKLEIK |
| 471 | CXCR5-1-115 | DIVMTQSPLSLPVSLGERASISCRSSQSLVNSDGNTYLEWYQQKP |
| | | GQSPKLLIYKASNRFSGVPDRFSGSGSGTDFTLKISRVEAEDVGV |
| | | YYCFQSSSVPLTFGQGTKLEIK |
| 472 | CXCR5-1-116 | DIVMTQSPLSLPVSLGERASISCRSSQSLVHSDGKTYLHWYQQKP |
| | | GQSPKLLIYKASNRFSGVPDRFSGSGSGTDFTLKISRVEAEDVGV |
| | | YYCQQGYSTPLTFGQGTKLEIK |
| 473 | CXCR5-1-117 | DIVMTQSPLSLPVSLGERASISCRSSQSLVHSNGNTYLAWYQQKP |
| | | GQSPKLLIYKASNRFSGVPDRFSGSGSGTDFTLKISRVEAEDVGV |
| | | YYCQQGSEVPLTFGQGTKLEIK |
| 474 | CXCR5-1-118 | DIVMTQSPLSLPVSLGERASISCRSSQSLVNSDGKTYLHWYQQKP |
| | | GQSPKLLIYKASNRFSGVPDRFSGSGSGTDFTLKISRVEAEDVGV |
| | | YYCQQGSHVPLTFGQGTKLEIK |
| 475 | CXCR5-1-119 | DIVMTQSPLSLPVSLGERASISCRSSQSLVHSNGNTYLHWYQQKP |
| | | GQSPKLLIYKVSNRFSGVPDRFSGSGSGTDFTLKISRVEAEDVGV |
| | | YYCQQGYSVPFTFGQGTKLEIK |
| 476 | CXCR5-1-120 | DIVMTQSPLSLPVSLGERASISCRSSQSLVNSDGKTYLEWYQQKP |
| | | GQSPKLLIYKVSNRASGVPDRFSGSGSGTDFTLKISRVEAEDVGV |
| | | YYCFQGYEYPLTFGQGTKLEIK |
| 477 | CXCR5-1-121 | DIVMTQSPLSLPVSLGERASISCRSSQSLVHSDGNTYLAWYQQKP |
| | | GQSPKLLIYKASNRASGVPDRFSGSGSGTDFTLKISRVEAEDVGV |
| | | YYCQQGYHYPLTFGQGTKLEIK |
| 478 | CXCR5-1-122 | DIVMTQSPLSLPVSLGERASISCRSSQSLVHSDGKTYLAWYQQKP |
| | | GQSPKLLIYKASNRASGVPDRFSGSGSGTDFTLKISRVEAEDVGV |
| | | YYCQQGYHVPFTFGQGTKLEIK |
| 479 | CXCR5-1-123 | DIVMTQSPLSLPVSLGERASISCRSSQSLVNSDGNTYLEWYQQKP |
| | | GQSPKLLIYKVSNRFSGVPDRFSGSGSGTDFTLKISRVEAEDVGV |
| | | YYCFQGSSYPFTFGQGTKLEIK |
| 480 | CXCR5-1-124 | DIVMTQSPLSLPVSLGERASISCRSSQSLVHSDGKTYLEWYQQKP |
| | | GQSPKLLIYKVSNRASGVPDRFSGSGSGTDFTLKISRVEAEDVGV |
| | | YYCFQSYHYPLTFGQGTKLEIK |
| 481 | CXCR5-1-125 | DIVMTQSPLSLPVSLGERASISCRSSQSLVHSDGKTYLAWYQQKP |
| | | GQSPKLLIYKVSNRFSGVPDRFSGSGSGTDFTLKISRVEAEDVGV |
| | | YYCQQSSHTPFTFGQGTKLEIK |
| 482 | CXCR5-1-126 | DIVMTQSPLSLPVSLGERASISCRSSQSLVHSDGKTYLHWYQQKP |
| | | GQSPKLLIYKASNRASGVPDRFSGSGSGTDFTLKISRVEAEDVGV |
| | | YYCQQGYEYPFTFGQGTKLEIK |
| 483 | CXCR5-1-127 | DIVMTQSPLSLPVSLGERASISCRSSQSLVHSNGKTYLAWYQQKP |
| | | GQSPKLLIYKVSNRFSGVPDRFSGSGSGTDFTLKISRVEAEDVGV |
| | | YYCQQGYEVPLTFGQGTKLEIK |
| 484 | CXCR5-1-128 | DIVMTQSPLSLPVSLGERASISCRSSQSLVNSNGKTYLAWYQQKP |
| | | GQSPKLLIYKASNRFSGVPDRFSGSGSGTDFTLKISRVEAEDVGV |
| | | YYCQQSYHYPFTFGQGTKLEIK |
| 485 | CXCR5-1-129 | DIVMTQSPLSLPVSLGERASISCRSSQSLVHSNGNTYLAWYQQKP |
| | | GQSPKLLIYKASNRFSGVPDRFSGSGSGTDFTLKISRVEAEDVGV |
| | | YYCQQGYSVPFTFGQGTKLEIK |
| 486 | CXCR5-1-130 | DIVMTQSPLSLPVSLGERASISCRSSQSLVNSNGKTYLHWYQQKP |
| | | GQSPKLLIYKVSNRFSGVPDRFSGSGSGTDFTLKISRVEAEDVGV |
| | | YYCFQSSHTPLTFGQGTKLEIK |
| 487 | CXCR5-1-131 | DIVMTQSPLSLPVSLGERASISCRSSQSLVHSDGNTYLAWYQQKP |
| | | GQSPKLLIYKVSNRFSGVPDRFSGSGSGTDFTLKISRVEAEDVGV |
| | | YYCFQSSSTPLTFGQGTKLEIK |
| 488 | CXCR5-1-132 | DIVMTQSPLSLPVSLGERASISCRSSQSLVNSDGNTYLAWYQQKP |
| | | GQSPKLLIYKVSNRFSGVPDRFSGSGSGTDFTLKISRVEAEDVGV |
| | | YYCFQSSEYPLTFGQGTKLEIK |
| 489 | CXCR5-1-133 | DIVMTQSPLSLPVSLGERASISCRSSQSLVHSNGKTYLAWYQQKP |
| | | GQSPKLLIYKASNRFSGVPDRFSGSGSGTDFTLKISRVEAEDVGV |
| | | YYCFQSYHTPFTFGQGTKLEIK |
| 490 | CXCR5-1-134 | DIVMTQSPLSLPVSLGERASISCRSSQSLVHSNGNTYLAWYQQKP |
| | | GQSPKLLIYKVSNRASGVPDRFSGSGSGTDFTLKISRVEAEDVGV |
| | | YYCQQSYSYPLTFGQGTKLEIK |
| 491 | CXCR5-1-135 | DIVMTQSPLSLPVSLGERASISCRSSQSLVNSDGKTYLAWYQQKP |
| | | GQSPKLLIYKASNRASGVPDRFSGSGSGTDFTLKISRVEAEDVGV |
| | | YYCFQSSHVPFTFGQGTKLEIK |
| 492 | CXCR5-1-136 | DIVMTQSPLSLPVSLGERASISCRSSQSLVNINGKTYLHWYQQKP |
| | | GQSPKLLIYKASNRFSGVPDRFSGSGSGTDFTLKISRVEAEDVGV |
| | | YYCQQGSETPFTFGQGTKLEIK |
| 493 | CXCR5-1-137 | DIVMTQSPLSLPVSLGERASISCRSSQSLVHSNGNTYLEWYQQKP |
| | | GQSPKLLIYKASNRASGVPDRFSGSGSGTDFTLKISRVEAEDVGV |
| | | YYCQQSSSYPFTFGQGTKLEIK |
| 494 | CXCR5-1-138 | DIVMTQSPLSLPVSLGERASISCRSSQSLVHSNGNTYLHWYQQKP |
| | | GQSPKLLIYKASNRASGVPDRFSGSGSGTDFTLKISRVEAEDVGV |
| | | YYCQQGSEYPFTFGQGTKLEIK |
| 495 | CXCR5-1-139 | DIVMTQSPLSLPVSLGERASISCRSSQSLVNSDGKTYLEWYQQKP |
| | | GQSPKLLIYKVSNRASGVPDRFSGSGSGTDFTLKISRVEAEDVGV |
| | | YYCFQGSSYPLTFGQGTKLEIK |
| 496 | CXCR5-1-140 | DIVMTQSPLSLPVSLGERASISCRSSQSLVNSNGNTYLEWYQQKP |
| | | GQSPKLLIYKASNRASGVPDRFSGSGSGTDFTLKISRVEAEDVGV |
| | | YYCFQGSEVPLTFGQGTKLEIK |
| 497 | CXCR5-1-141 | DIVMTQSPLSLPVSLGERASISCRSSQSLVNSDGNTYLAWYQQKP |
| | | GQSPKLLIYKASNRASGVPDRFSGSGSGTDFTLKISRVEAEDVGV |
| | | YYCQQSSEYPLTFGQGTKLEIK |
| 498 | CXCR5-1-142 | DIVMTQSPLSLPVSLGERASISCRSSQSLVNSNGNTYLHWYQQKP |
| | | GQSPKLLIYKASNRASGVPDRFSGSGSGTDFTLKISRVEAEDVGV |
| | | YYCQQSYHTPLTFGQGTKLEIK |
| 499 | CXCR5-1-143 | DIVMTQSPLSLPVSLGERASISCRSSQSLVHSNGKTYLEWYQQKP |
| | | GQSPKLLIYKASNRFSGVPDRFSGSGSGTDFTLKISRVEAEDVGV |
| | | YYCQQGSSYPLTFGQGTKLEIK |
| 500 | CXCR5-1-144 | DIVMTQSPLSLPVSLGERASISCRSSQSLVHSNGKTYLEWYQQKP |
| | | GQSPKLLIYKVSNRASGVPDRFSGSGSGTDFTLKISRVEAEDVGV |
| | | YYCQQSYSVPFTFGQGTKLEIK |
| 501 | CXCR5-1-145 | DIVMTQSPLSLPVSLGERASISCRSSQSLVHSDGKTYLHWYQQKP |
| | | GQSPKLLIYKVSNRASGVPDRFSGSGSGTDFTLKISRVEAEDVGV |
| | | YYCQQSSHYPLTFGQGTKLEIK |
| 502 | CXCR5-1-146 | DIVMTQSPLSLPVSLGERASISCRSSQSLVHSDGNTYLAWYQQKP |
| | | GQSPKLLIYKASNRFSGVPDRFSGSGSGTDFTLKISRVEAEDVGV |
| | | YYCQQGYEVPFTFGQGTKLEIK |
| 503 | CXCR5-1-147 | DIVMTQSPLSLPVSLGERASISCRSSQSLVHSDGKTYLHWYQQKP |
| | | GQSPKLLIYKVSNRASGVPDRFSGSGSGTDFTLKISRVEAEDVGV |
| | | YYCQQGSETPLTFGQGTKLEIK |
| 504 | CXCR5-1-148 | DIVMTQSPLSLPVSLGERASISCRSSQSLVNSDGKTYLAWYQQKP |
| | | GQSPKLLIYKASNRASGVPDRFSGSGSGTDFTLKISRVEAEDVGV |
| | | YYCFQGYNTPFTFGQGTKVEIK |
| 505 | CXCR5-1-149 | DIVMTQSPLSLPVSLGERASISCRSSQSLVNSNGKTYLEWYQQKP |
| | | GQSPKLLIYKASNRFSGVPDRFSGSGSGTDFTLKISRVEAEDVGV |
| | | YYCFQGYETPLTFGQGTKLEIK |
| 506 | CXCR5-1-150 | DIVMTQSPLSLPVSLGERASISCRSSQSLVNSDGNTYLAWYQQKP |
| | | GQSPKLLIYKASNRASGVPDRFSGSGSGTDFTLKISRVEAEDVGV |
| | | YYCFQSSETPFTFGQGTKLEIK |
| 507 | CXCR5-1-151 | DIVMTQSPLSLPVSLGERASISCRSSQSLVHSNGNTYLHWYQQKP |
| | | GQSPKLLIYKASNRASGVPDRFSGSGSGTDFTLKISRVEAEDVGV |
| | | YYCQQSYHYPFTFGQGTKLEIK |
| 508 | CXCR5-1-152 | DIVMTQSPLSLPVSLGERASISCRSSQSLVHSDGNTYLEWYQQKP |
| | | GQSPKLLIYKVSNRASGVPDRFSGSGSGTDFTLKISRVEAEDVGV |
| | | YYCFQSYSVPLTFGQGTKLEIK |
| 509 | CXCR5-1-153 | DIVMTQSPLSLPVSLGERASISCRSSQSLVNSDGKTYLAWYQQKP |
| | | GQSPKLLIYKVSNRASGVPDRFSGSGSGTDFTLKISRVEAEDVGV |
| | | YYCFQSYSVPFTFGQGTKLEIK |
| 510 | CXCR5-1-154 | DIVMTQSPLSLPVSLGERASISCRSSQSLVNSDGKTYLEWYQQKP |
| | | GQSPKLLIYKASNRASGVPDRFSGSGSGTDFTLKISRVEAEDVGV |
| | | YYCFQSYSTPFTFGQGTKLEIK |
| 511 | CXCR5-1-155 | DIVMTQSPLSLPVSLGERASISCRSSQSLVHSNGKTYLHWYQQKP |
| | | GQSPKLLIYKASNRASGVPDRFSGSGSGTDFTLKISRVEAEDVGV |
| | | YYCQQGSSVPLTFGQGTKLEIK |
| 512 | CXCR5-1-156 | DIVMTQSPLSLPVSLGERASISCRSSQSLVNSNGKTYLAWYQQKP |
| | | GQSPKLLIYKASNRASGVPDRFSGSGSGTDFTLKISRVEAEDVGV |
| | | YYCQQGSETPLTFGQGTKLEIK |
| 513 | CXCR5-1-157 | DIVMTQSPLSLPVSLGERASISCRSSQSLVHSNGKTYLAWYQQKP |
| | | GQSPKLLIYKASNRASGVPDRFSGSGSGTDFTLKISRVEAEDVGV |
| | | YYCFQGSEYPLTFGQGTKLEIK |
| 514 | CXCR5-1-158 | DIVMTQSPLSLPVSLGERASISCRSSQSLVHSDGKTYLHWYQQKP |
| | | GQSPKLLIYKVSNRFSGVPDRFSGSGSGTDFTLKISRVEAEDVGV |
| | | YYCFQGSETPLTFGQGTKLEIK |
| 515 | CXCR5-1-159 | DIVMTQSPLSLPVSLGERASISCRSSQSLVNSNGKTYLAWYQQKP |
| | | GQSPKLLIYKASNRFSGVPDRFSGSGSGTDFTLKISRVEAEDVGV |
| | | YYCQQSSEYPFTFGQGTKLEIK |
| 516 | CXCR5-1-160 | DIVMTQSPLSLPVSLGERASISCRSSQSLVHSNGKTYLEWYQQKP |
| | | GQSPKLLIYKVSNRFSGVPDRFSGSGSGTDFTLKISRVEAEDVGV |
| | | YYCQQSYHVPFTFGQGTKLEIK |
| 517 | CXCR5-1-161 | DIVMTQSPLSLPVSLGERASISCRSSQSLVHSDGKTYLHWYQQKP |
| | | GQSPKLLIYKASNRFSGVPDRFSGSGSGTDFTLKISRVEAEDVGV |
| | | YYCQQSSEVPFTFGQGTKLEIK |
| 518 | CXCR5-1-162 | RYSLTQSPLSLPVSLGERASISCRSSQSLVHSNGNTYLHWYQQKP |
| | | GQSPKLLIYKASNRASGVPDRFSGSGSGTDFTLKISRVEAEDVGV |
| | | YYCQQGSHTPLTFGQGTKLEIK |
| 519 | CXCR5-1-163 | DIVMTQSPLSLPVSLGERASISCRSSQSLVHSNGNTYLAWYQQKP |
| | | GQSPKLLIYKASNRFSGVPDRFSGSGSGTDFTLKISRVEAEDVGV |
| | | YYCQQSYSYPFTFGQGTKLEIK |
| 520 | CXCR5-1-164 | DIVMTQSPLSLPVSLGERASISCRSSQSLVHSNGNTYLHWYQQKP |
| | | GQSPKLLIYKASNRASGVPDRFSGSGSGTDFTLKISRVEAEDVGV |
| | | YYCQQSSHYPFTFGQGTKLEIK |
| 521 | CXCR5-1-165 | DIVMTQSPLSLPVSLGERASISCRSSQSLVNSDGNTYLAWYQQKP |
| | | GQSPKLLIYKASNRASGVPDRFSGSGSGTDFTLKISRVEAEDVGV |
| | | YYCFQSSETPFTFGQGTKLEIK |
| 522 | CXCR5-17-3 | DIQMTQSPSSLSASVGDRVTITCRTSQSISIYLNWYQQKPGKAPK |
| | | LLIYAASRVQSGVPSRFSGSGSGTDFTLTISSLQPEDFATYYCQE |
| | | SYSIPFTFGGGTKVEIK |
| 523 | CXCR5-17-11 | QSVLTQPPSVSAAPGQKVTISCSGSSSNIENNDVSWYQQLPGTAP |
| | | KLLIYQNNERPSGIPDRFSGSKSGTSATLGITGLQTGDEADYYCA |
| | | TWDRSLSVVFGGGTKLT |
| 524 | CXCR5-50-18 | DIQMTQSPSSLSASVGDRVTITCRASQSIYNYLNWYQQKPGKAPK |
| | | LLIYAASGLQSGVPSRFSGSGSGTDFTLTISSLQPEDFATYYCQQ |
| | | SFNTPLTFGGGTKVEIK |
| 525 | CXCR5-16 | DIVMTQAAPSVAVTPGASVSISCRSSKSLLHSSGKTYLYWFLQRP |
| | | GQSPQLLIYRLSSLASGVPDRFSGSGSGTAFTLRISRVEAEDVGV |
| | | YYCMQHLEYPYTFGGGTKLEIK |

TABLE 11
CXCR5 Variable Heavy Chain CDR's

| | SEQ | | SEQ | | SEQ | |
|---|---|---|---|---|---|---|
| | ID | | ID | | ID | |
| Variant | NO | CDR1 | NO | CDR2 | NO | CDR3 |
| CXCR5-1-1 | 526 | GFTFSDY | 663 | SPDGSI | 978 | KDVWVIFSTHDGAYGFDV |
| CXCR5-1-2 | 527 | GRAFIAY | 664 | SWSGGI | 979 | ASPGGAINYGRGYD |
| CXCR5-1-3 | 528 | GFTFSSY | 665 | SPNGGN | 980 | HDHDYYAFDY |
| CXCR5-1-4 | 529 | GGTFSSY | 666 | NPGDGY | 981 | HTSSNGVYSTWFAY |
| CXCR5-1-5 | 530 | GGTFSLY | 667 | SWSGGS | 982 | NESDAYN |
| CXCR5-1-6 | 531 | GFTFSSY | 668 | SYSGGE | 983 | DDDGGDAFDY |
| CXCR5-1-7 | 532 | GRAFIAY | 669 | SWSGGI | 984 | ASPGGAINYGRGYD |
| CXCR5-1-8 | 533 | GGTFSDY | 670 | NPYDGY | 985 | DYSSSFVFHAMDY |
| CXCR5-1-9 | 534 | GFTFSDY | 671 | SYDGSN | 986 | IRTNYFGFDY |
| CXCR5-1-10 | 535 | GRAFIAY | 672 | SWSGGI | 987 | ASPGGAINYGRGYD |
| CXCR5-1-11 | 536 | GRAFIAY | 673 | SWSGGI | 988 | ASPGGAINYGRGYD |
| CXCR5-1-12 | 537 | GFTFSDY | 674 | SYSGSE | 989 | HLTNYDPFDY |
| CXCR5-1-13 | 538 | GFTFSDY | 675 | SP | 990 | GDTNWFAFDY |
| CXCR5-1-14 | 539 | GFTFSNY | 676 | SPNGGN | 991 | ILTGGYPFDY |
| CXCR5-1-15 | 540 | GGTFSLY | 677 | SWSGGS | 992 | NESDAYN |
| CXCR5-1-16 | 541 | GFTFSDY | 678 | SYDGSN | 993 | HRHYGYPFDY |
| CXCR5-1-17 | 542 | GFTFSNY | 679 | SYSGGI | 994 | HRHYNYAFDY |
| CXCR5-1-18 | 543 | GGTFSDY | 680 | RPGDGY | 995 | FGHSGRSFAY |
| CXCR5-1-19 | 544 | GFTFSSY | 681 | SPSGGN | 996 | GKDDRLDYLGYYFDY |
| CXCR5-1-20 | 545 | GFTFSDY | 682 | SPDGGN | 997 | HLDGGDGFDY |
| CXCR5-1-21 | 546 | GFTFSDY | 683 | SYDGSE | 998 | DDRGYFGFDY |
| CXCR5-1-22 | 547 | GFTFSDY | 684 | SYSGSI | 999 | PSYLDSVYGHDGYYTLDV |
| CXCR5-1-23 | 548 | GGTFSSY | 685 | RPGDGY | 1000 | SLLPNTVTAYMDY |
| CXCR5-1-24 | 549 | GFTFSSY | 686 | SYDGGI | 1001 | DDDGWYPFDY |
| CXCR5-1-25 | 550 | GGTFSSY | 687 | RPYDGY | 1002 | HGYKSNYLSYMDY |
| CXCR5-1-26 | 551 | GFTFSSY | 688 | SYSGGN | 1003 | DDHGWYPFDY |
| CXCR5-1-27 | 552 | GFTFSDY | 689 | SPSGSI | 1004 | GRHNNFGFDY |
| CXCR5-1-28 | 553 | GFTFSNY | 690 | SYDGSI | 1005 | ILDYYFPFDY |
| CXCR5-1-29 | 554 | GGTFSNY | 691 | RPGNGY | 1006 | SESSFYVYQTAFAY |
| CXCR5-1-30 | 555 | GGTFSSY | 692 | RPGDGY | 1007 | SGLWYNVFNAMDY |
| CXCR5-1-31 | 556 | GFTFSDY | 693 | SPSGGN | 1008 | KSHYFGFWGNNGARTFDY |
| CXCR5-1-32 | 557 | GFTFSDY | 694 | SYDGSN | 1009 | GELNRGDRYGYRYHKHRGMDV |
| CXCR5-1-33 | 558 | GGTFSNY | 695 | RPNNGE | 1010 | DLYWNFGGYAMDY |
| CXCR5-1-34 | 559 | GGTFSDY | 696 | NPNDGY | 1011 | SFFYYHYGAFDY |
| CXCR5-1-35 | 560 | GFTFSSY | 697 | SYDGGN | 1012 | IDGYYIRWTYYHARTFDY |
| CXCR5-1-36 | 561 | GGTFSSY | 698 | RPNNGE | 1013 | PSRPSHYSAFSHPYYMDY |
| CXCR5-1-37 | 562 | GFTFSDY | 699 | SYSGSN | 1014 | GPQSWYGLWGQNFDY |
| CXCR5-1-38 | 563 | GFTFSNY | 700 | SPSGSE | 1015 | HLDNGFPFDY |
| CXCR5-1-39 | 564 | GFTFSDY | 701 | SYDGSI | 1016 | IRHRFILWRNYGARGMDY |
| CXCR5-1-40 | 565 | GFTFSSY | 702 | SPSGSE | 1017 | DRDRYLDLHRYPFDY |
| CXCR5-1-41 | 566 | GGTFSSY | 703 | NPNNGY | 1018 | LLSKSNNLHAMDY |
| CXCR5-1-42 | 567 | GGTFSSY | 704 | RPGNGY | 1019 | GGAYYYTSITSHGFQFDY |
| CXCR5-1-43 | 568 | GGTFSDY | 705 | RPYDGY | 1020 | STIGYDYGYYGFDY |
| CXCR5-1-44 | 569 | GFTFSDY | 706 | ANKYYA | 1021 | RVYWDGFYTQDYYYTLDV |
| CXCR5-1-45 | 570 | GFTFSDY | 707 | SYNGGI | 1022 | DTSSWTPLLTFYFDY |
| CXCR5-1-46 | 571 | GFTFSDY | 708 | SYDGSE | 1023 | HLHDNDAFDY |
| CXCR5-1-47 | 572 | GFTFSNY | 709 | SYSGGI | 1024 | HRTDGYPFDY |
| CXCR5-1-48 | 573 | GFTFSNY | 710 | SYSGGN | 1025 | KDGLYDRSGYRHARTFDY |
| CXCR5-1-49 | 574 | GFTFSDY | 711 | SPSGGE | 1026 | DEDYYYDGSRFNGGYYGPMDV |
| CXCR5-1-50 | 575 | GFTFSDY | 712 | SPSGGN | 1027 | RDYWYSVYTHRYARTFDV |
| CXCR5-1-51 | 576 | GFTFSNY | 713 | SYDGSI | 1028 | DRHGNYAFDY |
| CXCR5-1-52 | 577 | GGTFSSY | 714 | RPYDGY | 1029 | RGYSRDWFAY |
| CXCR5-1-53 | 578 | GGTFSDY | 715 | RPGDGE | 1030 | LFFSSDDFAFAFDY |
| CXCR5-1-54 | 579 | GFTFSDY | 716 | SYSGSN | 1031 | DLTGYYPFDY |
| CXCR5-1-55 | 580 | GFTFSDY | 717 | SPSGSN | 1032 | DDDGYLDYLRFNFDY |
| CXCR5-1-56 | 581 | GGTFSNY | 718 | RPNNGE | 1033 | LYGPNTVTYYMDY |
| CXCR5-1-57 | 582 | GGTFSSY | 719 | NPNNGE | 1034 | GSAYYHYYYYSHGGAFAY |
| CXCR5-1-58 | 583 | GFTFSSY | 720 | SPDGGN | 1035 | RVHWYGRYTHNYYYGLDV |
| CXCR5-1-59 | 584 | GGTFSSY | 721 | NPGDGY | 1036 | HESGYGVGAYGFAY |
| CXCR5-1-60 | 585 | GGTFSDY | 722 | NPYNGY | 1037 | PGEPYDTYITSFGFQMDY |
| CXCR5-1-61 | 586 | GFTFSNY | 723 | SYDGSE | 1038 | GRSDYYDLHTHNFDY |
| CXCR5-1-62 | 587 | GGTFSSY | 724 | NPGDGY | 1039 | LESKYDVGSAMDY |
| CXCR5-1-63 | 588 | GFTFSSY | 725 | SPSGGN | 1040 | ISVRYIRTGNDYARTMDY |
| CXCR5-1-64 | 589 | GFTFSSY | 726 | SYSGSN | 1041 | IDHWDGRWGYYHARTMDV |
| CXCR5-1-65 | 590 | GFTFSSY | 727 | SPNGGE | 1042 | GTSRYLPLHTYYFDY |
| CXCR5-1-66 | 591 | GFTFSDY | 728 | SYSGGE | 1043 | IRTYNYPFDY |
| CXCR5-1-67 | 592 | GGTFSNY | 729 | NPYNGY | 1044 | HYLWYYYFAAMDY |
| CXCR5-1-68 | 593 | GFTFSSY | 730 | SYSGSN | 1045 | IDTDNFAFDY |
| CXCR5-1-69 | 594 | GFTFSDY | 731 | SPDGGI | 1046 | DEDYYGIFYGQNHYFGFGMDV |
| CXCR5-1-70 | 595 | GGTFSSY | 732 | RPNNGY | 1047 | PSAYIDVSYTSFYGYFAY |
| CXCR5-1-71 | 596 | GFTFSSY | 733 | SPSGGN | 1048 | DRDYNFAFDY |
| CXCR5-1-72 | 597 | GFTFSSY | 734 | SPDGSN | 1049 | DRLHYGDSWRYNHHKYGGMDV |
| CXCR5-1-73 | 598 | GFTFSNY | 735 | SYDGGN | 1050 | IRDYGYGFDY |
| CXCR5-1-74 | 599 | GGTFSSY | 736 | RPYDGY | 1051 | SYYKHNNLAYMDY |
| CXCR5-1-75 | 600 | GGTFSSY | 737 | RPGNGE | 1052 | HLSKYFVTNAMDY |
| CXCR5-1-76 | 601 | GFTFSNY | 738 | SPDGGI | 1053 | IVGRDDRSGNDYYRTMDY |
| CXCR5-1-77 | 602 | GFTFSNY | 739 | SYSGGE | 1054 | IDHDNYGFDY |
| CXCR5-1-78 | 603 | GGTFSSY | 740 | RPYNGY | 1055 | DFFGNYVYSFWFDY |
| CXCR5-1-79 | 604 | GFTFSSY | 741 | SYDGGE | 1056 | DELYYYIGWGHDHHFHRGMDV |
| CXCR5-1-80 | 605 | GFTFSDY | 742 | SYSGSE | 1057 | GDRGYYSFWTHPFDY |
| CXCR5-1-81 | 606 | GFTFSDY | 743 | SPDGGE | 1058 | DRTNGFGFDY |
| CXCR5-1-82 | 607 | GFTFSDY | 744 | SPDGGN | 1059 | GELHRGSSTRYDFHYYRGMDV |
| CXCR5-1-83 | 608 | GFTFSDY | 745 | SYSGGI | 1060 | PSYYDSLWRHRYYRTFDV |
| CXCR5-1-84 | 609 | GFTFSSY | 746 | SPDGSI | 1061 | HRTDNFPFDY |
| CXCR5-1-85 | 610 | GGTFSNY | 747 | NPYNGE | 1062 | SPFGFTYYSTYFAY |
| CXCR5-1-86 | 611 | GFTFSNY | 748 | SPSGGI | 1063 | PRYLFGRTGNRYYYTLDV |
| CXCR5-1-87 | 612 | GGTFSSY | 749 | RPYDGY | 1064 | HYSDYTDTSYMDY |
| CXCR5-1-88 | 613 | GFTFSDY | 750 | SPDGGI | 1065 | DDTNNDPFDY |
| CXCR5-1-89 | 614 | GFTFSSY | 751 | SYSGSE | 1066 | GEGHYYDSTRQRFYFYFPMDV |
| CXCR5-1-90 | 615 | GFTFSDY | 752 | SPSGSN | 1067 | RRYRFGFWRQHHAYTFDV |
| CXCR5-1-91 | 616 | GGTFSNY | 753 | RPGNGE | 1068 | SYLSSYDLYAMDY |
| CXCR5-1-92 | 617 | GFTFSNY | 754 | SPSGSE | 1069 | DKDSNGILHGQNFDY |
| CXCR5-1-93 | 618 | GGTFSNY | 755 | RPGDGY | 1070 | GYNWARKLVY |
| CXCR5-1-94 | 619 | GFTFSSY | 756 | SYDGSE | 1071 | GKSGWYPLHGQNFDY |
| CXCR5-1-95 | 620 | GGTFSSY | 757 | RPNNGY | 1072 | HFIYYGGFSTGFDY |
| CXCR5-1-96 | 621 | GFTFSSY | 758 | SPNGGE | 1073 | GDQDNGGRLGYYFDY |
| CXCR5-1-97 | 622 | GFTFSNY | 759 | SYSGGI | 1074 | DRTNYFPFDY |
| CXCR5-1-98 | 623 | GFTFSDY | 760 | SPSGGI | 1075 | ISHYVGLWRHYYYRGFDV |
| CXCR5-1-99 | 624 | GGTFSDY | 761 | RPNNGE | 1076 | LTSRSTDGQFAFDY |
| CXCR5-1-100 | 625 | GFTFSDY | 762 | SYSGSN | 1077 | ISVYFDLWGYYHYYGLDY |
| CXCR5-1-101 | 626 | GGTFSSY | 763 | RPNDGE | 1078 | LTFRFTNGYGGFDY |
| CXCR5-1-102 | 627 | GFTFSNY | 764 | SPSGSI | 1079 | GRGYYYIGTGHRGHKHRPMDV |
| CXCR5-1-103 | 628 | GFTFSDY | 765 | SPNGGI | 1080 | DTDSRLPYHRQPFDY |
| CXCR5-1-104 | 629 | GGTFSNY | 766 | RPNNGY | 1081 | LYYSSYNLAAMDY |
| CXCR5-1-105 | 630 | GGTFSSY | 767 | NPGDGY | 1082 | FYYYFDKLVY |
| CXCR5-1-106 | 631 | GFTFSSY | 768 | AYITYYP | 1083 | GDDGNFPFDY |
| CXCR5-1-107 | 632 | GGTFSSY | 769 | RPN | 1084 | PGEYMDYEITYAPFQFAY |
| CXCR5-1-108 | 633 | GGTFSDY | 770 | RPGDGY | 1085 | FGHSGRSFAY |
| CXCR5-1-109 | 634 | GGTFSNY | 771 | NPGNGE | 1086 | DPIDSYYFAYGFDY |
| CXCR5-1-110 | 635 | GGTFSDY | 772 | NPNDGE | 1087 | HGAPMSVSYTSHPFQMDY |
| CXCR5-1-111 | 636 | GGTFSDY | 773 | RPGNGY | 1088 | FYYYGAWLDY |
| CXCR5-1-112 | 637 | GFTFSSY | 774 | SYDGSE | 1089 | PSHYYDLWTQYYAYGLDY |
| CXCR5-1-113 | 638 | GGTFSNY | 775 | RPNNGE | 1090 | HTISYGYSQTWFDY |
| CXCR5-1-114 | 639 | GGTFSNY | 776 | NPYDGY | 1091 | LTGYFDVFAYGFDY |
| CXCR5-1-115 | 640 | GFT | 777 | SYDGGSI | 1092 | DRGYYYDGTTYNFGKGFPMDV |
| CXCR5-1-116 | 641 | GGTFSDY | 778 | NPYDGY | 1093 | LSFGNDYFQYAFDY |
| CXCR5-1-117 | 642 | GFTFSSY | 779 | SPDGSN | 1094 | KRHYDIFYGQRGARTFDV |
| CXCR5-1-118 | 643 | GFTFSSY | 780 | SPNGGI | 1095 | DKSDYGIYWTQGFDY |
| CXCR5-1-119 | 644 | GGTFSSY | 781 | RPNNGY | 1096 | SFSSNGGYSGAFAY |
| CXCR5-1-120 | 645 | GFTFSDY | 782 | SYDGGE | 1097 | HLDYGYGFDY |
| CXCR5-1-121 | 646 | GFTFSSY | 783 | SYNGGN | 1098 | DRDNYYSSTGQYFHKGRPMDV |
| CXCR5-1-122 | 647 | GFTFSNY | 784 | SYSGSE | 1099 | GTDSYGDFYTFNFDY |
| CXCR5-1-123 | 648 | GFTFSNY | 785 | SYSGGN | 1100 | PDVRDILWRYYYYRGMDY |
| CXCR5-1-124 | 649 | GFTFSNY | 786 | SYDGSN | 1101 | DEGHYYDFYTHDGGYYGGMDV |
| CXCR5-1-125 | 650 | GFTFSDY | 787 | SYDGGI | 1102 | GEDYRYSFYGYYYYKYFPMDV |
| CXCR5-1-126 | 651 | GFTFSSY | 788 | | 1103 | ATSRWGPYYRQGFDY |
| CXCR5-1-127 | 652 | GFTFSNY | 789 | SPSGGE | 1104 | PRGLYSVYTNDHARGLDY |
| CXCR5-1-128 | 653 | GFTFSSY | 790 | SYNGGN | 1105 | GDDNNYAFDY |
| CXCR5-1-129 | 654 | GFTFSNY | 791 | SYSGGN | 1106 | DDRNGFPFDY |
| CXCR5-1-130 | 655 | GFTFSSY | 792 | SPNGGN | 1107 | GLHNWYAFDY |
| CXCR5-1-131 | 656 | GFTFSNY | 793 | SYSGGI | 1108 | IRDNYFPFDY |
| CXCR5-1-132 | 657 | GFTFSNY | 794 | SYDGSN | 1109 | IRHLFGFSTQDHARGFDV |
| CXCR5-1-133 | 658 | GFTFSDY | 795 | SYNGGN | 1110 | DELYRGSGWGYYGYYGYPMDV |
| CXCR5-1-134 | 659 | GFTFSNY | 796 | SPNGGI | 1111 | HDDNNFGFDY |
| CXCR5-1-135 | 660 | GGTFSSY | 797 | RPGNGE | 1112 | GYSYAAYLDY |
| CXCR5-1-136 | 661 | GFTFSNY | 798 | SPSGGI | 1113 | IRHGNYAFDY |
| CXCR5-1-137 | 662 | GFTFSNY | 799 | SYNGGI | 1114 | GRRGNDPFDY |
| CXCR5-1-138 | 663 | GGTFSSY | 800 | NPNDGY | 1115 | LFISYDDFNTAFDY |
| CXCR5-1-139 | 664 | GFTFSDY | 801 | SYDGSN | 1116 | GTQRRTDLHTYPFDY |
| CXCR5-1-140 | 665 | GFTFSSY | 802 | SPSGSE | 1117 | DPSRWTGWYRYPFDY |
| CXCR5-1-141 | 666 | GFTFSDY | 803 | SPSGSE | 1118 | IDRDYFAFDY |
| CXCR5-1-142 | 667 | GGTFSNY | 804 | RPNDGE | 1119 | STSYYYNYATWFAY |
| CXCR5-1-143 | 668 | GGTFSSY | 805 | RPNNGY | 1120 | DYYWYFVYSAIDY |
| CXCR5-1-144 | 669 | GFTFSSY | 806 | SPDGGE | 1121 | DRDDRGILWTYNFDY |
| CXCR5-1-145 | 670 | GFTFSDY | 807 | SYDGGI | 1122 | IFVLFSLTGQNYYRTLDY |
| CXCR5-1-146 | 671 | GFTFSNY | 808 | SYDGGN | 1123 | DDSDWTSLLRFNFDY |
| CXCR5-1-147 | 672 | GFTFSNY | 809 | SYDGSN | 1124 | HDRDGYAFDY |
| CXCR5-1-148 | 673 | GGTFSNY | 810 | RPGNGY | 1125 | LTSRFYNFQYYFAY |
| CXCR5-1-149 | 674 | GFTFSDY | 811 | SYSGSN | 1126 | GELYYYSGSYYDYGYYYGMDV |
| CXCR5-1-150 | 675 | GGTFSSY | 812 | RPNDGE | 1127 | DEYSYTYGYYMDY |
| CXCR5-1-151 | 676 | GFTFSSY | 813 | SYSGSN | 1128 | HLHDNFAFDY |
| CXCR5-1-152 | 677 | GFTFSSY | 814 | SPDGGI | 1129 | RVVLFDLTGYDYAYTFDY |
| CXCR5-1-153 | 678 | GFTFSSY | 815 | SYDGGN | 1130 | GEDNRYISSGYDYYYHGPMDV |
| CXCR5-1-154 | 679 | GGTFSNY | 816 | RPNNGE | 1131 | HSRPYDTSYTYFGFAMDY |
| CXCR5-1-155 | 680 | GGTFSNY | 817 | RLNNGY | 1132 | LPFGSGYSSTAFDY |
| CXCR5-1-156 | 681 | GGTFSSY | 818 | RPNDGY | 1133 | HSEPSDVSITSFPYTFDY |
| CXCR5-1-157 | 682 | GGTFSSY | 819 | NPNDGY | 1134 | HGSPNTYYYYMDY |
| CXCR5-1-158 | 683 | GGTFSNY | 820 | RPNNGE | 1135 | GYGSGAAFDY |
| CXCR5-1-159 | 684 | GFTFSDY | 821 | SPSGSE | 1136 | DEDHYYIFWGHNYHYHRPMDV |
| CXCR5-1-160 | 685 | GGTFSNY | 822 | NPGDGY | 1137 | DYSWHDYLNYMDY |
| CXCR5-1-161 | 686 | GFTFSNY | 823 | SPNGGN | 1138 | DEGHYYSGWTFNHHKYGGMDV |
| CXCR5-1-162 | 687 | GFTFSNY | 824 | SYSGGN | 1139 | IDVWDSFWGYDHARGLDV |
| CXCR5-1-163 | 688 | GGTFSDY | 825 | RPGDGE | 1140 | HFGRFTVFQGGFAY |
| CXCR5-1-164 | 689 | GGTFSDY | 826 | NPGDGY | 1141 | LYSSNFGYSAMDY |
| CXCR5-1-165 | 690 | GFTFSSY | 827 | SYNGGE | 1142 | DEGHRGDSLRFDFHKHFPMDV |
| CXCR5-2-1 | 691 | GSTISDR | 828 | IGDA | 1143 | ALQYCSPTSCYVDDYFYYMDV |
| CXCR5-2-2 | 692 | GFTFSTY | 829 | SGSGSI | 1144 | GPEWTPPGDYFYYMDD |
| CXCR5-2-3 | 693 | GFSLDDY | 830 | GSDGS | 1145 | WFGDYNF |
| CXCR5-2-4 | 694 | GRGFSRY | 831 | TPINWGGRGT | 1146 | DPPG |
| CXCR5-2-5 | 695 | GNIAAIN | 832 | SWSSGS | 1147 | DRGGL |
| CXCR5-2-6 | 696 | DLSFSFY | 833 | NWSGT | 1148 | EDDYYDGTGYYQYYGMDV |
| CXCR5-2-7 | 697 | GFTVSNY | 834 | RWSGGI | 1149 | DRGGS |
| CXCR5-2-8 | 698 | GFTLDYY | 835 | NWSGDT | 1150 | EGCSSTSCYLDP |
| CXCR5-2-9 | 699 | GFTFSTY | 836 | SGSGSI | 1151 | TLSPYAMDV |
| CXCR5-2-10 | 700 | GFSFDDDY | 837 | DWNGNS | 1152 | GPEWTPPGDYFYYMDD |
| CXCR5-2-11 | 701 | GGTFSIY | 838 | STHSI | 1153 | YLEMSPGEYFDN |
| CXCR5-2-12 | 702 | GFTFSTY | 839 | SGSGSI | 1154 | YWRTGDWFDP |
| CXCR5-2-13 | 703 | GITFRRY | 840 | SSSGAL | 1155 | DRTGSGWFRDV |
| CXCR5-2-14 | 526 | GIPSIR | 841 | SRSGET | 1156 | SGLDDGYYPED |
| CXCR5-2-15 | 527 | GSIDSIH | 842 | SWTGGS | 1157 | DPPG |
| CXCR5-2-16 | 528 | GSTISDR | 843 | IGDA | 1158 | DMGG |
| CXCR5-2-17 | 529 | GMTTIG | 844 | SWSGGL | 1159 | VYYDSSGYNDY |
| CXCR5-2-18 | 530 | GSTISDR | 845 | IGDA | 1160 | GPEWTPPGDYFYYMDD |
| CXCR5-2-19 | 531 | GSIDSIH | 846 | SWTGGS | 1161 | GMVRGVDF |
| CXCR5-2-20 | 532 | GRTFSDY | 847 | NWNGDS | 1162 | LFAQYSDYDYVAE |
| CXCR5-2-21 | 533 | GRTFFSY | 848 | RWSGGS | 1163 | GRPVPR |
| CXCR5-2-22 | 534 | GNIFRIE | 849 | HSSGS | 1164 | SDYDVVSGLTNDYLYYLDD |
| CXCR5-2-23 | 535 | GFNFDDY | 850 | SSGGN | 1165 | TSYYYSSGSSFSGRLDYLDD |
| CXCR5-2-24 | 536 | GFPFSEY | 851 | AWGDGI | 1166 | IFVGMDV |
| CXCR5-2-25 | 537 | GFPFDDY | 852 | TRSGKT | 1167 | VYYDSSGYNDY |
| CXCR5-2-26 | 538 | GFPFDDY | 853 | SWSAGS | 978 | VRDFWGGYDIDH |
| CXCR5-2-27 | 539 | GFNLDDYA | 854 | TWSGGL | 979 | DRGGS |
| CXCR5-2-28 | 540 | GFGID | 855 | SWSGDS | 980 | AGGPYYDLSTGSSGHLDY |
| CXCR5-2-29 | 541 | GFDFDNFDDY | 856 | NRSGDT | 981 | AGPNYYDSDTRGDY |
| CXCR5-2-30 | 542 | GFNFDDY | 857 | STDVDS | 982 | AEGYWYFDL |
| CXCR5-2-31 | 543 | GFGFGSY | 858 | TSSDGR | 983 | APYTSVAGRAYYYYYGMDV |
| CXCR5-2-32 | 544 | GFDFDNFDDY | 859 | NRSGDT | 984 | WFGDYNF |
| CXCR5-2-33 | 545 | GFPFSIW | 860 | RWSGAS | 985 | LDILGGPDTVGAFDL |
| CXCR5-2-34 | 546 | GFPFSEY | 861 | AWGDGI | 986 | DMGG |
| CXCR5-2-35 | 547 | GFSFDDY | 862 | RWSGGI | 987 | VARDRGYNYDSD |
| CXCR5-2-36 | 548 | GFPLDDY | 863 | AWGDGS | 988 | TFKTGYRSGYY |
| CXCR5-2-37 | 549 | GFPLDDY | 864 | SSEGT | 989 | DQSAYGQTVFFDS |
| CXCR5-2-38 | 550 | GFPLDYY | 865 | SRSGGS | 990 | DPDDYGDYTFDY |
| CXCR5-2-39 | 551 | GFSFDDDY | 866 | SRSGGD | 991 | GMVRGVDF |
| CXCR5-2-40 | 552 | GFSFDDDY | 867 | DWNGNS | 992 | WIHMKGGFLDY |
| CXCR5-2-41 | 553 | GFSFDDDY | 868 | DWNGNS | 993 | ADCSGGVCNAY |
| CXCR5-2-42 | 554 | GFAFSRY | 869 | TPGGN | 994 | TSWGLVY |
| CXCR5-2-43 | 555 | GFSFDDY | 870 | SFGGN | 995 | TSYYYSSGSSFSGRLDYLDD |
| CXCR5-2-44 | 556 | GFSLDDY | 871 | GSDGS | 996 | ALQYCSPTSCYVDDYFYYMDV |
| CXCR5-2-45 | 557 | GFSLDDY | 872 | GSDGS | 997 | GFSSGWYGWDS |
| CXCR5-2-46 | 558 | GFSLDDY | 873 | SRSGNV | 998 | WFGDYNF |
| CXCR5-2-47 | 559 | GFSLDDY | 874 | AWSSDF | 999 | ASPGRYCSGRSCYFDWYFHL |
| CXCR5-2-48 | 560 | GFSLDYY | 875 | SWIIGS | 1000 | ALQYCSPTSCYVDDYFYYMDV |
| CXCR5-2-49 | 561 | GFSLDYY | 876 | SWIIGS | 1001 | VNPSDYYDSRGYPDY |
| CXCR5-2-50 | 562 | GFAFSTA | 877 | TRGS | 1002 | TLSPYAMDV |
| CXCR5-2-51 | 563 | GDTFNWY | 878 | TADGI | 1003 | DREAYSYGYNDY |
| CXCR5-2-52 | 564 | GFAFDDY | 879 | RWSGGI | 1004 | EETLQQLLRAYC |
| CXCR5-2-53 | 565 | GFTDDYY | 880 | SWSGGS | 1005 | GPYGGASYFTV |
| CXCR5-2-54 | 566 | GFTFENY | 881 | NWNGAS | 1006 | DHPNYYYGMDV |
| CXCR5-2-55 | 567 | GFTFSTH | 882 | YPSG | 1007 | EGPRVDLNYDFWSPDYYYYMDV |
| CXCR5-2-56 | 568 | DLSFSFY | 883 | TSGGI | 1008 | EDDYYDGTGYYQYYGMDV |
| CXCR5-2-57 | 569 | GRGFSRY | 884 | TPINWGGRGT | 1009 | EDDYYDGTGYYQYYGMDV |
| CXCR5-2-58 | 570 | GSTFSKA | 885 | SSSGIS | 1010 | GGGPHYYYYYYMDV |
| CXCR5-2-59 | 571 | GSTFSSY | 886 | NYSGGS | 1011 | EGEYSSSWYYYYYGMDV |
| CXCR5-2-60 | 572 | GYFASWY | 887 | SRGGMTSLGDS | 1012 | DRPDYYYYYGMDV |
| CXCR5-2-61 | 573 | GCTVSIN | 888 | SWSGGS | 1013 | LFAQYSDYDYVAE |
| CXCR5-2-62 | 574 | GDIFSNY | 889 | GSDGS | 1014 | AVGATSDDPFDM |
| CXCR5-2-63 | 575 | GDIGSIN | 890 | RWSGGI | 1015 | SGGNYGDYVV |
| CXCR5-2-64 | 576 | GDIGSIN | 891 | TPINWGGRGT | 1016 | RGSGVATRVY |
| CXCR5-2-65 | 577 | GDIGSIN | 892 | RWSGGI | 1017 | TRHDYSNVY |
| CXCR5-2-66 | 578 | GDIGSIN | 893 | SRSGGT | 1018 | VTSGADAFDI |
| CXCR5-2-67 | 579 | GDISSIV | 894 | RWSEDR | 1019 | DQGREDDFWSGYDEPRDV |
| CXCR5-2-68 | 580 | GDISSIV | 895 | RWSEDR | 1020 | TFKTGYRSGYY |
| CXCR5-2-69 | 581 | GDTFNWY | 896 | DWSGSS | 1021 | LEFNYYDSRQLR |
| CXCR5-2-70 | 582 | GDTFNWY | 897 | SRSGDT | 1022 | ASSDYGDVSGP |
| CXCR5-2-71 | 583 | GDTFNWY | 898 | SRSGDT | 1023 | TGSSSPDSYMDV |
| CXCR5-2-72 | 584 | GDTFNWY | 899 | SRSGSI | 1024 | DVGNNWYADS |
| CXCR5-2-73 | 585 | GDTFNWY | 900 | SWSEDN | 1025 | AAQDYGDSTFDF |
| CXCR5-2-74 | 586 | GDTFNWY | 901 | TNGGS | 1026 | CSGGSCNY |
| CXCR5-2-75 | 587 | GDTFSSY | 902 | TWSGGI | 1027 | DLYYDSSGYYGG |
| CXCR5-2-76 | 588 | GDTFSWY | 903 | SNSGLS | 1028 | AYCSGGSCYDY |
| CXCR5-2-77 | 589 | GDTFSWY | 904 | SRSGGT | 1029 | VMESGYDYLDY |
| CXCR5-2-78 | 590 | GDTFSWY | 905 | SSSGEV | 1030 | IVLVAVGELTDY |
| CXCR5-2-79 | 591 | GERAFSNY | 906 | TSGGT | 1031 | GPEWTPPGDYFYYMDD |
| CXCR5-2-80 | 592 | GFSLDYY | 907 | DWSGGT | 1032 | GPEWTPPGDYFYYMDD |
| CXCR5-2-81 | 593 | GFTFSTY | 908 | SGSGSI | 1033 | DWQSLVRGVSIDQ |
| CXCR5-2-82 | 594 | GFTDDYY | 909 | DTSGI | 1034 | GQLRYFDWLLDYYFDY |
| CXCR5-2-83 | 595 | GFTFSSY | 910 | SSSGVT | 1035 | DPPG |
| CXCR5-2-84 | 596 | GFTFSTS | 911 | SMSGDD | 1036 | GNYYMDV |
| CXCR5-2-85 | 597 | GFTFSTY | 912 | NWDSAR | 1037 | DQH |
| CXCR5-2-86 | 598 | GFTFSTY | 913 | SGGGSI | 1038 | GPEWTPPGDYFYYMDD |
| CXCR5-2-87 | 599 | GFTFSTY | 914 | SGSGA | 1039 | GPEWTPPGDYFYYMDV |
| CXCR5-2-88 | 600 | GFTFSTY | 915 | SGSGSI | 1040 | GSYGGYV |
| CXCR5-2-89 | 601 | GFTFSTY | 916 | SGSGSI | 1041 | QYCAAGSCYDK |
| CXCR5-2-90 | 602 | GFTFSTY | 917 | SGSGSI | 1042 | AERGSERAY |
| CXCR5-2-91 | 603 | GFTFSTY | 918 | SGSGSI | 1043 | AGPNYYDSDTRGDY |
| CXCR5-2-92 | 604 | GFTFSTY | 919 | SGSGSI | 1044 | DGDFWSGYRDY |
| CXCR5-2-93 | 605 | GSIYSLD | 920 | SRSGSI | 1045 | DHYVWGTFDP |
| CXCR5-2-94 | 606 | GFTFSTY | 921 | SGSGSI | 1046 | GPYGGASYFTV |
| CXCR5-2-95 | 607 | GFTFSTY | 922 | SGSGSI | 1047 | GVGYCGGMGCHEGDY |
| CXCR5-2-96 | 608 | GFTFSTY | 923 | SGSGSI | 1048 | PYCSSTSCYSS |
| CXCR5-2-97 | 609 | GFTFSTY | 924 | SGSGSI | 1049 | QMCGGGDCYIH |
| CXCR5-2-98 | 610 | GFTFSTY | 925 | SGSGSI | 1050 | VYYDSSGYYDY |
| CXCR5-2-99 | 611 | GFTFSTY | 926 | SGSGSI | 1051 | LWAGYDGDYFNY |
| CXCR5-2-100 | 612 | GFTFSTY | 927 | SGSGSI | 1052 | SKLVGSTYVDY |
| CXCR5-2-101 | 613 | GFTFSTY | 928 | SGSGSI | 1053 | WMGTYGDDY |
| CXCR5-2-102 | 614 | GFTFSTY | 929 | SSSGGS | 1054 | AGYEDY |
| CXCR5-2-103 | 615 | GFTFSTY | 930 | SSSGGS | 1055 | GPEWTPPGDYFYYMDD |
| CXCR5-2-104 | 616 | GFTFSTY | 931 | YSDGS | 1056 | VESEDLLVDSLIY |
| CXCR5-2-105 | 617 | GFTFSTY | 932 | NWNGDS | 1057 | GPEWTPPGDYFYYMDD |
| CXCR5-2-106 | 618 | GFTIDDY | 933 | NSDGT | 1058 | VVYGSDSFDDF |
| CXCR5-2-107 | 619 | GFTLDAY | 934 | NSGGS | 1059 | AYDFWSGPVY |
| CXCR5-2-108 | 620 | GFTLDAY | 935 | NSGGS | 1060 | PNLRYTYGYDY |
| CXCR5-2-109 | 621 | GFTLDAY | 936 | SKSDGS | 1061 | VYYDSSGYNDY |
| CXCR5-2-110 | 622 | GFTLDAY | 937 | SRSGN | 1062 | NRLTGDSSQVF |
| CXCR5-2-111 | 623 | GFTFSSY | 938 | SSSGVT | 1063 | DQSAYGQTVFFDS |
| CXCR5-2-112 | 624 | GFTFSSY | 939 | SSSGVT | 1064 | GPYYYDSSGYYGPNDY |
| CXCR5-2-113 | 625 | GFTDDYY | 940 | SWSGSN | 1065 | ALQYCSPTSCYVDDYFYYMDV |
| CXCR5-2-114 | 626 | GFTDGID | 941 | SWSGGI | 1066 | AGDTRNDYNYGAY |
| CXCR5-2-115 | 627 | GFTFDDT | 942 | GSDGS | 1067 | GAQWEQRTYDS |
| CXCR5-2-116 | 628 | GFTFDDY | 943 | SSSDGS | 1068 | ALDGYSGS |
| CXCR5-2-117 | 629 | GFTFDDY | 944 | RWSDGT | 1069 | LVVPANTYFYYAMDV |
| CXCR5-2-118 | 630 | GFTFDDY | 945 | RWSDGT | 1070 | DGADTAPIYGMAV |
| CXCR5-2-119 | 631 | GFTFDDY | 946 | SRSPGV | 1071 | EPGPADYRDY |
| CXCR5-2-120 | 632 | GFTFDDY | 947 | STGGDT | 1072 | DLSGRGDVSEYEYD |
| CXCR5-2-121 | 633 | GFTFDDY | 948 | SSSDKD | 1073 | VANDYGNYEPS |
| CXCR5-2-122 | 634 | GFTFDGY | 949 | SWDGRN | 1074 | AGDTRNDYNYGAY |
| CXCR5-2-123 | 635 | GFTFDRS | 950 | GSDGT | 1075 | DYYDSSGYYYV |
| CXCR5-2-124 | 636 | GFTFDRSY | 951 | NWSLTR | 1076 | GTFDVLRFLEWRL |
| CXCR5-2-125 | 637 | GFTFDYY | 952 | SWNGGS | 1077 | YYDSSGYSQDFDY |
| CXCR5-2-126 | 638 | GFTFEDY | 953 | SGSGSI | 1078 | GPEWTPPGDYFYYMDD |
| CXCR5-2-127 | 639 | GFTFEDY | 954 | SSSGIS | 1079 | EGCSSTSCYLDP |
| CXCR5-2-128 | 640 | GFTFEDY | 955 | SSGGT | 1080 | TSYYGDFE |
| CXCR5-2-129 | 641 | GFTFGHY | 956 | NRSGDT | 1081 | GPEWTPPGDYFYYMDD |
| CXCR5-2-130 | 642 | GFTFRRY | 957 | RWSGGI | 1082 | GPEWTPPGDYFYYMDD |
| CXCR5-2-131 | 643 | GFTFRRY | 958 | RWSGGI | 1083 | GRSRGTSGTTAD |
| CXCR5-2-132 | 644 | GFTFRSY | 959 | SGSDGS | 1084 | ASSDYGDVSGP |
| CXCR5-2-133 | 645 | GFTFSDY | 960 | ASMGGL | 1085 | LFAQYSDYDYVAE |
| CXCR5-2-134 | 646 | GFTFSDY | 961 | ASMGGL | 1086 | SDYDVVSGLTNDYLYYLDD |
| CXCR5-2-135 | 647 | GFTFSEY | 962 | AWSSDF | 1087 | VGLGSCSTTSCFDY |
| CXCR5-2-136 | 648 | GFTFSGN | 963 | RWSGGQI | 1088 | GPTGPPRFFDF |
| CXCR5-2-137 | 649 | GFTFSNY | 964 | TSGGS | 1089 | GASY |
| CXCR5-2-138 | 650 | GFTFSRY | 965 | NYSGGS | 1090 | VGAAGAVF |
| CXCR5-2-139 | 651 | GFTFSSD | 966 | SGTGTI | 1091 | GSGGGVDY |
| CXCR5-2-140 | 652 | GFTFSSDDY | 967 | NWSGKD | 1092 | ANKYYYDYYGVDV |
| CXCR5-2-141 | 653 | GYTYTTY | 968 | YWSGKD | 1093 | GPDMIRSWYAWFDP |
| CXCR5-2-142 | 654 | GNIFINN | 969 | NRSGGA | 1094 | LFGSPSSSADYYYFDL |
| CXCR5-2-143 | 655 | GSIFSIN | 970 | SWSAGS | 1095 | DRCGGDCNFSVLDWFDP |
| CXCR5-2-144 | 656 | GFNFDDY | 971 | SSGGN | 1096 | IISSTTGTDYFQN |
| CXCR5-2-145 | 657 | GRTFSNT | 972 | SWSGDN | 1097 | AGGYDYVWGSYPSDY |
| CXCR5-2-146 | 658 | GRTGTIY | 973 | SWSDGS | 1098 | SDYDVVSGLTNDYLYYLDD |
| CXCR5-2-147 | 659 | GRTPSII | 974 | SSEGT | 1099 | VGEQTEYVDGTGYDYFYAMDV |
| CXCR5-2-148 | 660 | GSIDNIH | 975 | TWSGDS | 1100 | SPGIRGPINH |
| CXCR5-2-149 | 661 | GSIDSIH | 976 | SWTGGS | 1101 | DFDYGDYWERDAFDI |
| CXCR5-2-150 | 662 | GSIDSIH | 977 | SWTGGS | 1102 | GRVGVYGDYLFDH |

TABLE 12
CXCR5 Variable Light Chain CDR's

| | SEQ | | SEQ | | SEQ | |
|---|---|---|---|---|---|---|
| | ID | | ID | | ID | |
| Variant | NO | CDR1 | NO | CDR2 | NO | CDR3 |
| CXCR5-1-1 | 1103 | RSSQSLVHSDGNTYLE | 1268 | KVSNRASG | 1329 | QQSSETPLT |
| CXCR5-1-2 | 1104 | RSSQSLVHSNGNTYLA | 1269 | KASNRASG | 1330 | QQSSSYPFT |
| CXCR5-1-3 | 1105 | RSSQSLVHSDGNTYLA | 1270 | KASNRFSG | 1331 | QQGSETPLT |
| CXCR5-1-4 | 1106 | RSSQSLVHSNGNTYLA | 1271 | KVSNRASG | 1332 | QQGYSTPLT |
| CXCR5-1-5 | 1107 | RSSQSLVHSDGKTYLE | 1272 | KASNRASG | 1333 | FQSSSTPFT |
| CXCR5-1-6 | 1108 | RSSQSLVNSDGKTYLH | 1273 | KASNRASG | 1334 | FQGSEYPFT |
| CXCR5-1-7 | 1109 | RSSQSLVNSDGNTYLE | 1274 | KASNRASG | 1335 | FQGSEYPFT |
| CXCR5-1-8 | 1110 | RSSQSLVHSNGNTYLH | 1275 | KVSNRASG | 1336 | FQSYHYPLT |
| CXCR5-1-9 | 1111 | RSSQSLVNSDGNTYLH | 1276 | KASNRASG | 1337 | QQGSSTPLT |
| CXCR5-1-10 | 1112 | RSSQSLVNSNGKTYLA | 1277 | KASNRFSG | 1338 | QQSSHVPFT |
| CXCR5-1-11 | 1113 | RSSQSLVNSNGKTYLA | 1278 | KASNRFSG | 1339 | QQGYHTPLT |
| CXCR5-1-12 | 1114 | RSSQSLVNSDGKTYLH | 1279 | KVSNRASG | 1340 | QQGYETPFT |
| CXCR5-1-13 | 1115 | RSSQSLVNSDGKTYLE | 1280 | KASNRFSG | 1341 | FQSSSVPLT |
| CXCR5-1-14 | 1116 | RSSQSLVNSNGKTYLA | 1281 | KVSNRASG | 1342 | QQSSEYPFT |
| CXCR5-1-15 | 1117 | RSSQSLVHSNGKTYLH | 1282 | KASNRASG | 1343 | FQGYHTPLT |
| CXCR5-1-16 | 1118 | RSSQSLVNSNGNTYLH | 1283 | KVSNRASG | 1344 | FQGSHYPFT |
| CXCR5-1-17 | 1119 | RSSQSLVHSDGNTYLH | 1284 | KVSNRASG | 1345 | QQGYETPFT |
| CXCR5-1-18 | 1120 | RSSQSLVHSNGKTYLA | 1285 | KASNRASG | 1346 | QQSYHVPFT |
| CXCR5-1-19 | 1121 | RSSQSLVNSDGNTYLH | 1286 | KASNRASG | 1347 | QQSSHTPFT |
| CXCR5-1-20 | 1122 | RSSQSLVNSDGKTYLE | 1287 | KASNRASG | 1348 | FQGSHYPFT |
| CXCR5-1-21 | 1123 | RSSQSLVNSDGNTYLH | 1288 | KVSNRFSG | 1349 | FQGYETPLT |
| CXCR5-1-22 | 1124 | RSSQSLVNSDGNTYLH | 1289 | KASNRASG | 1350 | QQSSHVPFT |
| CXCR5-1-23 | 1125 | RSSQSLVNSDGNTYLE | 1290 | KVSNRFSG | 1351 | FQGYHTPFT |
| CXCR5-1-24 | 1126 | RSSQSLVNSNGKTYLA | 1291 | KVSNRASG | 1352 | QQSYHTPFT |
| CXCR5-1-25 | 1127 | RSSQSLVNSDGNTYLA | 1292 | KASNRASG | 1353 | FQGSSTPLT |
| CXCR5-1-26 | 1128 | RSSQSLVNSNGNTYLH | 1293 | KVSNRFSG | 1354 | FQSYSTPFT |
| CXCR5-1-27 | 1129 | RSSQSLVHSNGNTYLH | 1294 | KASNRFSG | 1355 | QQSYSTPLT |
| CXCR5-1-28 | 1130 | RSSQSLVNSNGKTYLA | 1295 | KASNRASG | 1356 | QQSYEVPLT |
| CXCR5-1-29 | 1131 | RSSQSLVHSNGKTYLH | 1296 | KASNRASG | 1357 | QQSSSYPFT |
| CXCR5-1-30 | 1132 | RSSQSLVHSDGKTYLE | 1297 | KVSNRASG | 1358 | FQGSHYPFT |
| CXCR5-1-31 | 1133 | RSSQSLVNSDGKTYLH | 1298 | KVSNRASG | 1359 | QQGYSTPFT |
| CXCR5-1-32 | 1134 | RSSQSLVHSDGKTYLA | 1299 | KASNRFSG | 1360 | QQSYHVPFT |
| CXCR5-1-33 | 1135 | RSSQSLVHSDGKTYLE | 1300 | KASNRFSG | 1361 | QQSSHYPLT |
| CXCR5-1-34 | 1136 | RSSQSLVNSNGKTYLA | 1301 | KVSNRASG | 1362 | QQGSHVPFT |
| CXCR5-1-35 | 1137 | RSSQSLVNSDGNTYLH | 1302 | KASNRASG | 1363 | QQSSSTPFT |
| CXCR5-1-36 | 1138 | RSSQSLVNSNGNTYLH | 1303 | KASNRASG | 1364 | QQGYHYPLT |
| CXCR5-1-37 | 1139 | RSSQSLVNSNGKTYLA | 1304 | KASNRASG | 1365 | QQGSEYPLT |
| CXCR5-1-38 | 1140 | RSSQSLVHSNGNTYLE | 1305 | KVSNRASG | 1366 | QQGSSVPFT |
| CXCR5-1-39 | 1141 | RSSQSLVNSNGNTYLA | 1306 | KASNRASG | 1367 | FQGYEVPFT |
| CXCR5-1-40 | 1142 | RSSQSLVNSNGNTYLE | 1307 | KVSNRFSG | 1368 | QQSSEYPLT |
| CXCR5-1-41 | 1143 | RSSQSLVHSNGKTYLE | 1308 | KASNRASG | 1369 | FQGSHYPLT |
| CXCR5-1-42 | 1144 | RSSQSLVHSNGNTYLH | 1309 | KVSNRFSG | 1370 | QQSSSYPFT |
| CXCR5-1-43 | 1145 | RSSQSLVHSNGNTYLH | 1310 | KASNRASG | 1371 | FQSYEYPFT |
| CXCR5-1-44 | 1146 | RSSQSLVHSNGKTYLH | 1311 | KASNRASG | 1372 | FQSSSTPFT |
| CXCR5-1-45 | 1147 | RSSQSLVHSDGKTYLH | 1312 | KASNRFSG | 1373 | QQSYHYPFT |
| CXCR5-1-46 | 1148 | RSSQSLVHSDGNTYLA | 1313 | KVSNRASG | 1374 | FQGSHYPFT |
| CXCR5-1-47 | 1149 | RSSQSLVHSNGNTYLA | 1314 | KASNRFSG | 1375 | QQSYSYPLT |
| CXCR5-1-48 | 1150 | RSSQSLVNSNGNTYLA | 1315 | KVSNRASG | 1376 | FQSYHTPLT |
| CXCR5-1-49 | 1151 | RSSQSLVNSDGNTYLA | 1316 | KASNRFSG | 1377 | FQSYEVPLT |
| CXCR5-1-50 | 1152 | RSSQSLVHSDCKTYLA | 1317 | KASNRFSG | 1378 | QQGSEYPFT |
| CXCR5-1-51 | 1153 | RSSQSLVHSDGNTYLE | 1318 | KASNRASG | 1379 | QQSSSVPLT |
| CXCR5-1-52 | 1154 | RSSQSLVNSDGKTYLA | 1319 | KVSNRFSG | 1380 | QQGSHVPFT |
| CXCR5-1-53 | 1155 | RSSQSLVNSDGKTYLE | 1320 | KVSNRASG | 1381 | QQSYSVPLT |
| CXCR5-1-54 | 1156 | RSSQSLVNSNGKTYLA | 1321 | KVSNRFSG | 1382 | QQSYSVPLT |
| CXCR5-1-55 | 1157 | RSSQSLVNSNGNTYLA | 1322 | KVSNRASG | 1383 | FQSSHTPFT |
| CXCR5-1-56 | 1158 | RSSQSLVNSDGNTYLE | 1323 | KASNRASG | 1384 | FQGSSTPLT |
| CXCR5-1-57 | 1159 | RSSQSLVHSNGKTYLA | 1324 | KASNRFSG | 1385 | QQSSSYPFT |
| CXCR5-1-58 | 1160 | RSSQSLVHSDGNTYLA | 1325 | KASNRFSG | 1386 | QQSYEYPFT |
| CXCR5-1-59 | 1161 | RSSQSLVNSDGKTYLA | 1326 | KASNRASG | 1387 | FQSSHTPLT |
| CXCR5-1-60 | 1162 | RSSQSLVNSDGKTYLH | 1327 | KVSNRFSG | 1388 | QQSSETPFT |
| CXCR5-1-61 | 1163 | RSSQSLVNSNGNTYLA | 1328 | KASNRASG | 1389 | FQSYSVPLT |
| CXCR5-1-62 | 1164 | RSSQSLVNSDGKTYLE | 1329 | KASNRFSG | 1390 | FQSYETPLT |
| CXCR5-1-63 | 1165 | RSSQSLVHSNGNTYLA | 1330 | KASNRFSG | 1391 | FQSSSTPFT |
| CXCR5-1-64 | 1166 | RSSQSLVHSNGNTYLH | 1331 | KASNRASG | 1392 | QQSYHYPFT |
| CXCR5-1-65 | 1167 | RSSQSLVHSDGNTYLA | 1332 | KASNRFSG | 1393 | QQESSTPFT |
| CXCR5-1-66 | 1168 | RSSQSLVHSDGKTYLE | 1333 | KASNRFSG | 1394 | QQSYHYPLT |
| CXCR5-1-67 | 1169 | RSSQSLVNSDGKTYLH | 1334 | KASNRASG | 1395 | QQSYHTPLT |
| CXCR5-1-68 | 1170 | RSSQSLVHSDGNTYLA | 1335 | KVSNRASG | 1396 | QQSYEYPFT |
| CXCR5-1-69 | 1171 | RSSQSLVHSDGKTYLH | 1336 | KVSNRFSG | 1397 | QQGSSTPLT |
| CXCR5-1-70 | 1172 | RSSQSLVHSNGKTYLH | 1337 | KVSNRFSG | 1398 | QQGSSIPLT |
| CXCR5-1-71 | 1173 | RSSQSLVHSDGKTYLA | 1338 | KASNRASG | 1399 | FQSSSTPLT |
| CXCR5-1-72 | 1174 | RSSQSLVHSDGKTYLH | 1339 | KVSNRASG | 1400 | QQSYHYPLT |
| CXCR5-1-73 | 1175 | RSSQSLVNSDGNTYLA | 1340 | KASNRFSG | 1401 | FQGYETPLT |
| CXCR5-1-74 | 1176 | RSSQSLVHSNGNTYLA | 1341 | KVSNRASG | 1402 | QQGYHTPLT |
| CXCR5-1-75 | 1177 | RSSQSLVHSDGNTYLH | 1342 | KASNRASG | 1403 | QQSSHVPFT |
| CXCR5-1-76 | 1178 | RSSQSLVNSDGKTYLA | 1343 | KASNRFSG | 1404 | QQGYSYPLT |
| CXCR5-1-77 | 1179 | RSSQSLVNSNGNTYLE | 1344 | KVSNRFSG | 1405 | QQGSHVPFT |
| CXCR5-1-78 | 1180 | RSSQSLVNSNGKTYLA | 1345 | KVSNRFSG | 1406 | QQGYSVPLT |
| CXCR5-1-79 | 1181 | RSSQSLVHSNGKTYLE | 1346 | KVSNRASG | 1407 | FQSYSVPFT |
| CXCR5-1-80 | 1182 | RSSQSLVHSDGNTYLA | 1347 | KVSNRASG | 1408 | FQSYSYPLT |
| CXCR5-1-81 | 1183 | RSSQSLVNSNGKTYLA | 1348 | KASNRFSG | 1409 | QQSYHYPLT |
| CXCR5-1-82 | 1184 | RSSQSLVNSNSNTYLE | 1349 | KASNRFSG | 1410 | QQSSHVPFT |
| CXCR5-1-83 | 1185 | RSSQSLVNSNGNTYLH | 1350 | KVSNRASG | 1411 | QQGSHVPFT |
| CXCR5-1-84 | 1186 | RSSQSLVHSNGKTYLH | 1351 | KASNRASG | 1412 | QQGYHVPFT |
| CXCR5-1-85 | 1187 | RSSQSLVNSDGNTYLE | 1352 | KASNRASG | 1413 | QQSYHVPFT |
| CXCR5-1-86 | 1188 | RSSQSLVNSDGNTYLH | 1353 | KVSNRASG | 1414 | QQGYEYPFT |
| CXCR5-1-87 | 1189 | RSSQSLVHSNGKTYLA | 1354 | KASNRFSG | 1415 | QQSYSYALT |
| CXCR5-1-88 | 1190 | RSSQSLVNSDGKTYLH | 1355 | KASNRASG | 1416 | QQGSEVPFT |
| CXCR5-1-89 | 1191 | RSSQSLVHSDGKTYLA | 1356 | KVSNRASG | 1417 | QQSYSVPFT |
| CXCR5-1-90 | 1192 | RSSQSLVHSDGKTYLH | 1357 | KVSNRFSG | 1418 | FQGYSYPFT |
| CXCR5-1-91 | 1193 | RSSQSLVHSDGNTYLA | 1358 | KVSNRASG | 1419 | QQGSSYPLT |
| CXCR5-1-92 | 1194 | RSSQSLVNSDGNTYLH | 1359 | KVSNRFSG | 1420 | QQSYHTPFT |
| CXCR5-1-93 | 1195 | RSSQSLVNSNGNTYLH | 1360 | KASNRASG | 1421 | QQGYEYPLT |
| CXCR5-1-94 | 1196 | RSSQSLVHSDGNTYLA | 1361 | KVSNRASG | 1422 | QQSYSYPLT |
| CXCR5-1-95 | 1197 | RSSQSLVHSNGNTYLH | 1362 | KASNRASG | 1423 | QQGSEYPFT |
| CXCR5-1-96 | 1198 | RSSQSLVHSNGKTYLE | 1363 | KASNRASG | 1424 | FQSSSTPFT |
| CXCR5-1-97 | 1199 | RSSQSLVHSDGKTYLA | 1364 | KVSNRFSG | 1425 | QQSYSYPLT |
| CXCR5-1-98 | 1200 | RSSQSLVHSDGNTYLE | 1365 | KVSNRASG | 1426 | QQGYSVPFT |
| CXCR5-1-99 | 1201 | RSSQSLVNSDGKTYLE | 1366 | KASNRFSG | 1427 | QQSYSVPFT |
| CXCR5-1-100 | 1202 | RSSQSLVHSNGNTYLH | 1367 | KVSNRASG | 1428 | FQSSSYPFT |
| CXCR5-1-101 | 1203 | RSSQSLVNSNGNTYLH | 1368 | KASNRASG | 1429 | QQGYHVPFT |
| CXCR5-1-102 | 1204 | RSSQSLVHSDGNTYLE | 1369 | KVSNRFSG | 1430 | QQSYSTPFT |
| CXCR5-1-103 | 1205 | RSSQSLVNSNGKTYLE | 1370 | KVSNRASG | 1431 | QQSYHYPFT |
| CXCR5-1-104 | 1206 | RSSQSLVHSDGNTYLH | 1371 | KVSNRFSG | 1432 | QQSSHYPLT |
| CXCR5-1-105 | 1207 | RSSQSLVHSDGKTYLA | 1268 | KASNRFSG | 1433 | FQGSHTPFT |
| CXCR5-1-106 | 1208 | RSSQSLVHSDGKTYLH | 1269 | KASNRFSG | 1434 | FQGSSVPLT |
| CXCR5-1-107 | 1209 | RSSQSLVNSNGNTYLE | 1270 | KASNRFSG | 1435 | FQSYHVPFT |
| CXCR5-1-108 | 1210 | RSSQSLVHSNGKTYLA | 1271 | KASNRLSG | 1436 | QQSYSTPFT |
| CXCR5-1-109 | 1211 | RSSQSLVNSNGNTYLH | 1272 | KASNRASG | 1437 | QQGSEYPFT |
| CXCR5-1-110 | 1212 | RSSQSLVNSDGKTYLH | 1273 | KVSNRFSG | 1438 | QQSSHVPFT |
| CXCR5-1-111 | 1213 | RSSQSLVNSDGNTYLA | 1274 | KASNRASG | 1439 | QQGYSTPFT |
| CXCR5-1-112 | 1214 | RSSQSLVNSDGNTYLH | 1275 | KASNRASG | 1440 | QQGSEVPLT |
| CXCR5-1-113 | 1215 | RSSQSLVHSNGKTYLA | 1276 | KASNRFSG | 1441 | FQGYSTPLT |
| CXCR5-1-114 | 1216 | RSSQSLVHSNGKTYLH | 1277 | KVSNRASG | 1442 | FQSSEVPFT |
| CXCR5-1-115 | 1217 | RSSQSLVNSDGNTYLE | 1278 | KASNRFSG | 1443 | FQSSSVPLT |
| CXCR5-1-116 | 1218 | RSSQSLVHSDGKTYLH | 1279 | KASNRFSG | 1444 | QQGYSTPLT |
| CXCR5-1-117 | 1219 | RSSQSLVHSNGNTYLA | 1280 | KASNRFSG | 1445 | QQGSEVPLT |
| CXCR5-1-118 | 1220 | RSSQSLVNSDGKTYLH | 1281 | KASNRFSG | 1446 | QQGSHVPLT |
| CXCR5-1-119 | 1221 | RSSQSLVHSNGNTYLH | 1282 | KVSNRFSG | 1447 | QQGYSVPFT |
| CXCR5-1-120 | 1222 | RSSQSLVNSDGKTYLE | 1283 | KVSNRASG | 1448 | FQGYEYPLT |
| CXCR5-1-121 | 1223 | RSSQSLVHSDGNTYLA | 1284 | KASNRASG | 1449 | QQGYHYPLT |
| CXCR5-1-122 | 1224 | RSSQSLVHSDGKTYLA | 1285 | KASNRASG | 1450 | QQGYHVPFT |
| CXCR5-1-123 | 1225 | RSSQSLVNSDGNTYLE | 1286 | KVSNRFSG | 1451 | FQGSSYPFT |
| CXCR5-1-124 | 1226 | RSSQSLVHSDGKTYLE | 1287 | KVSNRASG | 1452 | FQSYHYPLT |
| CXCR5-1-125 | 1227 | RSSQSLVHSDGKTYLA | 1288 | KVSNRFSG | 1453 | QQSSHTPFT |
| CXCR5-1-126 | 1228 | RSSQSLVHSDGKTYLH | 1289 | KASNRASG | 1454 | QQGYEYPFT |
| CXCR5-1-127 | 1229 | RSSQSLVHSNGKTYLA | 1290 | KVSNRFSG | 1455 | QQGYEVPLT |
| CXCR5-1-128 | 1230 | RSSQSLVNSNGKTYLA | 1291 | KASNRFSG | 1456 | QQSYHYPFT |
| CXCR5-1-129 | 1231 | RSSQSLVHSNGNTYLA | 1292 | KASNRFSG | 1457 | QQGYSVPFT |
| CXCR5-1-130 | 1232 | RSSQSLVNSNGKTYLH | 1293 | KVSNRFSG | 1458 | FQSSHTPLT |
| CXCR5-1-131 | 1233 | RSSQSLVHSDGNTYLA | 1294 | KVSNRFSG | 1459 | FQSSSTPLT |
| CXCR5-1-132 | 1234 | RSSQSLVNSDGNTYLA | 1295 | KVSNRFSG | 1460 | FQSSEYPLT |
| CXCR5-1-133 | 1235 | RSSQSLVHSNGKTYLA | 1296 | KASNRFSG | 1461 | FQSYHTPFT |
| CXCR5-1-134 | 1236 | RSSQSLVHSNGNTYLA | 1297 | KVSNRASG | 1462 | QQSYSYPLT |
| CXCR5-1-135 | 1237 | RSSQSLVNSDGKTYLA | 1298 | KASNRASG | 1463 | FQSSHVPFT |
| CXCR5-1-136 | 1238 | RSSQSLVNINGKTYLH | 1299 | KASNRFSG | 1464 | QQGSETPFT |
| CXCR5-1-137 | 1239 | RSSQSLVHSNGNTYLE | 1300 | KASNRASG | 1465 | QQSSSYPFT |
| CXCR5-1-138 | 1240 | RSSQSLVHSNGNTYLH | 1301 | KASNRASG | 1466 | QQGSEYPFT |
| CXCR5-1-139 | 1241 | RSSQSLVNSDGKTYLE | 1302 | KVSNRASG | 1467 | FQGSSYPLT |
| CXCR5-1-140 | 1242 | RSSQSLVNSNGNTYLE | 1303 | KASNRASG | 1468 | FQGSEVPLT |
| CXCR5-1-141 | 1243 | RSSQSLVNSDGNTYLA | 1304 | KASNRASG | 1469 | QQSSEYPLT |
| CXCR5-1-142 | 1244 | RSSQSLVNSNGNTYLH | 1305 | KASNRASG | 1470 | QQSYHTPLT |
| CXCR5-1-143 | 1245 | RSSQSLVHSNGKTYLE | 1306 | KASNRFSG | 1471 | QQGSSYPLT |
| CXCR5-1-144 | 1246 | RSSQSLVHSNGKTYLE | 1307 | KVSNRASG | 1472 | QQSYSVPFT |
| CXCR5-1-145 | 1247 | RSSQSLVHSDGKTYLH | 1308 | KVSNRASG | 1473 | QQSSHYPLT |
| CXCR5-1-146 | 1248 | RSSQSLVHSDGNTYLA | 1309 | KASNRFSG | 1474 | QQGYEVPFT |
| CXCR5-1-147 | 1249 | RSSQSLVHSDGKTYLH | 1310 | KVSNRASG | 1475 | QQGSETPLT |
| CXCR5-1-148 | 1250 | RSSQSLVNSDGKTYLA | 1311 | KASNRASG | 1476 | FQGYNTPFT |
| CXCR5-1-149 | 1251 | RSSQSLVNSNGKTYLE | 1312 | KASNRFSG | 1477 | FQGYETPLT |
| CXCR5-1-150 | 1252 | RSSQSLVNSDGNTYLA | 1313 | KASNRASG | 1478 | FQSSETPFT |
| CXCR5-1-151 | 1253 | RSSQSLVHSNGNTYLH | 1314 | KASNRASG | 1479 | QQSYHYPFT |
| CXCR5-1-152 | 1254 | RSSQSLVHSDGNTYLE | 1315 | KVSNRASG | 1480 | FQSYSVPLT |
| CXCR5-1-153 | 1255 | RSSQSLVNSDGKTYLA | 1316 | KVSNRASG | 1481 | FQSYSVPFT |
| CXCR5-1-154 | 1256 | RSSQSLVNSDGKTYLE | 1317 | KASNRASG | 1482 | FQSYSTPFT |
| CXCR5-1-155 | 1257 | RSSQSLVHSNGKTYLH | 1318 | KASNRASG | 1483 | QQGSSVPLT |
| CXCR5-1-156 | 1258 | RSSQSLVNSNGKTYLA | 1319 | KASNRASG | 1484 | QQGSETPLT |
| CXCR5-1-157 | 1259 | RSSQSLVHSNGKTYLA | 1320 | KASNRASG | 1485 | FQGSEYPLT |
| CXCR5-1-158 | 1260 | RSSQSLVHSDGKTYLH | 1321 | KVSNRFSG | 1486 | FQGSETPLT |
| CXCR5-1-159 | 1261 | RSSQSLVNSNGKTYLA | 1322 | KASNRFSG | 1487 | QQSSEYPFT |
| CXCR5-1-160 | 1262 | RSSQSLVHSNGKTYLE | 1323 | KVSNRFSG | 1488 | QQSYHVPFT |
| CXCR5-1-161 | 1263 | RSSQSLVHSDGKTYLH | 1324 | KASNRFSG | 1489 | QQSSEVPFT |
| CXCR5-1-162 | 1264 | RSSQSLVHSNGNTYLH | 1325 | KASNRASG | 1490 | QQGSHTPLT |
| CXCR5-1-163 | 1265 | RSSQSLVHSNGNTYLA | 1326 | KASNRFSG | 1491 | QQSYSYPFT |
| CXCR5-1-164 | 1266 | RSSQSLVHSNGNTYLH | 1327 | KASNRASG | 1492 | QQSSHYPFT |
| CXCR5-1-165 | 1267 | RSSQSLVNSDGNTYLA | 1328 | KASNRASG | 1493 | FQSSETPFT |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. An antibody or antibody fragment comprising a variable domain, heavy chain region (V_{H}) and a variable domain, light chain region (V_{L}), wherein V_{H} comprises complementarity determining regions CDRH1, CDRH2, and CDRH3, wherein V_{L} comprises complementarity determining regions CDRL1, CDRL2, and CDRL3, and wherein (a) an amino acid sequence of CDRH1 is as set forth in any one of SEQ ID NOs: 526-703 and 1494-1555; (b) an amino acid sequence of CDRH2 is as set forth in any one of SEQ ID NOs: 704-977 and 1556-1558; (c) an amino acid sequence of CDRH3 is as set forth in any one of SEQ ID NOs: 978-1167 and 1559-1650; (d) an amino acid sequence of CDRL1 is as set forth in any one of SEQ ID NOs: 1168-1267 and 1651-1652; (e) an amino acid sequence of CDRL2 is as set forth in any one of SEQ ID NOs: 1268-1371 and 1653; and (f) an amino acid sequence of CDRL3 is as set forth in any one of SEQ ID NOs: 1372-1493 and 1654-1666.

2. The antibody or antibody fragment of claim 1, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

3. The antibody or antibody fragment of claim 1, wherein the antibody or antibody fragment thereof is chimeric or humanized.

4. The antibody or antibody fragment of claim 1, wherein the antibody or antibody fragment has an EC50 less than about 25 nanomolar in a cAMP assay.

5-6. (canceled)

7. The antibody or antibody fragment of claim 1, wherein the antibody or antibody fragment binds to a chemokine receptor with a K_{D} of less than 100 nM.

8-10. (canceled)

11. The antibody or antibody fragment of claim 1, wherein the antibody or antibody fragment is an agonist of a chemokine receptor.

12. The antibody or antibody fragment of claim 1, wherein the antibody or antibody fragment is an antagonist of a chemokine receptor.

13. The antibody or antibody fragment of claim 1, wherein the antibody or antibody fragment is an allosteric modulator of a chemokine receptor.

14. The antibody or antibody fragment of claim 13, wherein the allosteric modulator of a chemokine receptor is a negative allosteric modulator.

15. The antibody or antibody fragment of claim 11, wherein the chemokine receptor is CXCR4.

16. The antibody or antibody fragment of claim 11, wherein the chemokine receptor is CXCR5.

17. An antibody or antibody fragment comprising a variable domain, heavy chain region (V_{H}) and a variable domain, light chain region (V_{L}), wherein the V_{H} comprises an amino acid sequence at least about 90% identical to a sequence as set forth in any one of SEQ ID NOs: 24-28 or 34-356, and wherein the V_{L} comprises an amino acid sequence at least about 90% identical to a sequence as set forth in any one of SEQ ID NOs: 29-33 or 357-525.

18-23. (canceled)

24. The antibody or antibody fragment of claim 17, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

25-60. (canceled)

61. A method of treating a disease or disorder comprising administering the antibody or antibody fragment of claim 1.

62. The method of claim 61, wherein the disease or disorder affects homeostasis.

63. The method of claim 61, wherein the disease or disorder characterized by hematopoietic stem cell migration.

64. The method of claim 61, wherein the disease or disorder is a solid cancer or a hematologic cancer.

65. The method of claim 61, wherein the disease or disorder is gastric cancer, breast cancer, colorectal cancer, lung cancer, prostate cancer, hepatocellular carcinoma, leukemia, or lymphoma.

66. (canceled)

67. The method of claim 61, wherein the disease or disorder is caused by a virus.

68-69. (canceled)

70. A nucleic acid composition comprising: a) a first nucleic acid encoding a variable domain, heavy chain region (VH) comprising an amino acid sequence at least about 90% identical to a sequence as set forth in any one of SEQ ID NOs: 24-28 or 34-356; b) a second nucleic acid encoding a variable domain, light chain region (VL) comprising at least about 90% identical to a sequence as set forth in any one of SEQ ID NOs: 29-33 or 357-525; and an excipient.

71-76. (canceled)

77. A nucleic acid composition comprising: a nucleic acid encoding a variable domain, heavy chain region (VH) comprising an amino acid sequence at least about 90% identical to a sequence as set forth in any one of SEQ ID NOs: 24-28 or 34-356; and an excipient.

78-82. (canceled)
